(19) United States
(12) Patent Application Publication
(10) Pub. No.: US 2024/0201167 A1
(43) Pub. Date: Jun. 20, 2024

(54) THE IMMUNOMODULATORY LIGAND B7-1 MEDIATES SYNAPTIC REMODELING BY P75NTR

(71) Applicant: Albert Einstein College of Medicine, The Bronx, NY (US)

(72) Inventors: Barbara Hempstead, New York, NY (US); Francis Lee, New York, NY (US); Roshelle Smith, Bronx, NY (US); Victor Danelon, New York, NY (US); Steven Almo, Pelham, NY (US); Nicholas Morano, Staten Island, NY (US); Sarah Garrett, Mahopac, NY (US)

(21) Appl. No.: 18/556,488

(22) PCT Filed: Apr. 21, 2022
(86) PCT No.: PCT/US22/25730

§ 371 Date: Oct. 20, 2023

Related U.S. Application Data
(60) Provisional application No. 63/178,163, filed on Apr. 22, 2021.

(51) Int. Cl. G01N 33/50; A61P 25/00; C07K 14/705

(52) U.S. Cl. CPC G01N 33/5008; A61P 25/00; C07K 14/70521; C07K 2319/30

(57) ABSTRACT

As described herein, binding of the B7-1 protein with the neuronal cell surface p75 neurotrophin receptor protein triggers loss of synaptic connections. Methods and compositions are also described for treatment of neurological diseases and conditions (including pain) and for identifying therapeutic agents useful for treatment of neurological diseases and conditions (including pain).

### RELATED APPLICATIONS

This application claims benefit of priority to the filing date of U.S. Provisional Application Ser. No. 63/178,163, filed Apr. 22, 2021, the contents of which are specifically incorporated by reference herein in their entirety.

### INCORPORATION BY REFERENCE OF SEQUENCE LISTING PROVIDED AS A TEXT FILE

A Sequence Listing is provided herewith as a text file, “2232107.txt” created on Apr. 21, 2022 and having a size of 28,672 bytes. The contents of the text file are incorporated by reference herein in their entirety.

### BACKGROUND

Neurological conditions with different etiologies and symptoms can result in progressive degeneration and/or death of neuronal cells. Successful treatment of such neurological conditions remains a significant challenge in part because the cellular or molecular basis of the condition or disease is poorly understood. Currently approved therapies for CNS demyelinating diseases, such as multiple sclerosis (MS), are primarily immunomodulatory, and typically do not have direct effects on CNS repair. Similarly, drugs for other neurodegenerative diseases such as Alzheimer's disease and Parkinson's disease do not address the neuronal death and loss of function, but rather ameliorate associated symptoms.

Therefore, additional therapies that prevent and/or neurological conditions are needed.

### SUMMARY

As described herein, binding of the B7-1 protein with the neuronal cell surface p75 neurotrophin receptor triggers loss of synaptic connections. Methods and compositions are described herein that can inhibit such B7-1:p75 neurotrophin receptor binding that are useful for treatment of neurological diseases and conditions. Methods are also described herein for identifying therapeutic agents useful for treatment of neurological diseases and conditions.

For example, a method is described herein that involves incubating one or more test agents with B7-1 and p75 neurotrophin receptor and measuring whether one or more of the test agents reduces B7-1 binding to p75 neurotrophin receptor. The one or more of the test agents can, for example, be small molecules, antibodies, antibody fragments, antibody-derived constructs, Fc-fusion proteins, proteins, peptides, aptamers, peptide aptamers, nucleic acid aptamers, darpins, nanobodies, affinity reagents, liposomes displaying at least one test agent, or cells expressing at least one test agent on the cells' surface.

In some cases, the B7-1 or p75 neurotrophin receptor used is in soluble form. In some cases, B7-1 and p75 neurotrophin receptor are expressed separately on different cells, or at least one of B7-1 or p75 neurotrophin receptor is linked to different beads or carriers.

Such methods can also include selecting one or more of the test agents that reduce B7-1 binding to p75 neurotrophin receptor by at least 25%, or at least 50%, or at least 75% compared to a control assay mixture of the B7-1 and the p75 neurotrophin receptor without the one or more test agents, to thereby identify at least one B7-1 blocking agent. The methods can further include incubating at least one B7-1 blocking agent with B7-1 in the presence of CD28 or CTLA-4, and measuring whether at least one of the B7-1 blocking agents reduces B7-1 binding to CD28 or CTLA-4. For example, the method can also include selecting one or more of the B7-1 blocking agents that does not significantly reduce B7-1 binding to CD28 or CTLA-4 to thereby identify at least one B7-1-specific blocking agent.

In addition, the method can include incubating at least one B7-1-specific blocking agent in a culture comprising B7-1 (e.g., as soluble B7-1 or as cell-bound B7-1) and neurons that express p75 neurotrophin receptor, and measuring synaptic puncta density of the neurons that express p75 neurotrophin receptor. Such methods can also include selecting at least one B7-1-specific blocking agent that maintains higher levels of synaptic puncta density compared to a control culture comprising B7-1 (e.g., as soluble B7-1 or as cell-bound B7-1) and neurons that express p75 neurotrophin receptor without the B7-1-specific blocking agent, to thereby identify a B7-1 inhibitor. For example, useful test agents (e.g., B7-1 or p75 blocking agents) can facilitate retention of at least 25%, or at least 50%, or at least 75%, or at least 80%, or at least 90%, or at least 95% more dendritic processes of synaptic punta than in a control assay mixture of the B7-1 and the p75 neurotrophin receptor without the one or more test agents.

B7-1 inhibitors that are identified by the methods described herein can be administered to an animal. In some cases, the animal can be an animal model of a neuronal condition or disease. The effects of such administration on the animal model can be observed and measured. For example, the animal model can be monitored for reduced symptoms of Alzheimer's disease, cognitive impairment, multiple sclerosis, stroke, neuronal injury, traumatic neural injury, spinal cord injury, pain (e.g., acute pain, chronic pain, neuropathic pain, nociceptive pain, radicular pain, thermal pain, or combinations thereof), lupus, Parkinson's disease, anxiety, schizophrenia, manic depression, delirium, dementia, mental retardation, Huntington's disease, or Tourette's syndrome, compared to a model animal that did not receive the at least one B7-1-specific blocking agent or at least one B7-1 inhibitor.

Also described herein are modified p75 neurotrophin receptor proteins that have one or more replacements, deletions or insertions into a binding domain for B7-1 protein, nerve growth factor (NGF), proNGF, neurotrophin-3 (NT3), or receptor-type tyrosine-protein phosphatase F (PTPRF). The PTPRF protein is a membrane protein that when upregulated is thought to be associated with the occurrence and development of immune-mediated demyelinating diseases. In some cases the modified p75 neurotrophin receptor protein is soluble p75 neurotrophin receptor protein that selectively binds B7-1, but does not bind NGF, proNGF, NT3, or PTPRF. Such a modified p75 neurotrophin receptor protein can be administered to a subject, for example, a subject that has a neuronal condition or disease.

Also described herein are modified B7-1 proteins with one or more replacements, deletions or insertions in a binding domain for p75 neurotrophin receptor protein. For example, the modified B7-1 protein can have one or more replacements, deletions, or insertions into amino acid positions corresponding to one or more of positions 36, 39, 40, 43, 49, 63, 65, 82, 120, 121, 127, or 139 of SEQ ID NO:1. Such modified B7-1 proteins can be administered to a subject, for example, a subject that has a neuronal condition or disease.

Also described herein are antibodies that bind to a p75 neurotrophin receptor peptide epitope, wherein the peptide sequence includes 3-10 amino acids at any of p75 neurotrophin receptor positions 36, 37, 38, 49, 95, 104, 136, 137, 147, 150, 162, 165, 171, or 182 of SEQ ID NO:8. For example, the peptide epitope sequence can include 3-10 amino acids at any of p75 neurotrophin receptor positions F136, S137, S137, E147, P150, P150, L165, or R182 of SEQ ID NO:8. Such antibodies can be human or humanized antibodies. The antibodies can be administered to a subject, for example, a subject that has a neuronal condition or disease.

Also described herein are antibodies that have at least one cdr region that binds to a B7-1 peptide epitope. For example, the at least one cdr region that binds to a B7-1 peptide epitope. The peptide epitope can have at least 3-10 amino acids at any of B7-1 positions 36, 39, 40, 43, 49, 63, 65, 82, 120, 121, 127, or 139 of SEQ ID NO:1. Such antibodies can be human or humanized antibodies. The antibodies can be administered to a subject, for example, a subject that has a neuronal condition or disease.

Also described herein are peptides and compositions of such peptides, where the peptide has a sequence that includes LSRKIGRT (SEQ ID NO:11) or LSRKAVRRA (SEQ ID NO:12). Such a peptide can inhibit B7-1:p75 binding. In some cases, the peptide also inhibits binding of p75^{NTR} to NT3, NGF, proNGF, BDNF, proBDNF, NT4 or a combination thereof.

### DESCRIPTION OF THE FIGURES

FIG. 1A-1D illustrate the screening methods and the results identifying that B7-1 (CD80) binds to p75NTR. FIG. 1A is a schematic diagram of the high throughput cell:cell screening method used to initially identify that B7-1 (CD80) binds to p75 neurotrophin receptor (p75NTR). As illustrated, cells were transfected with expression cassettes expressing labeled receptors or labeled ligands, receptor-expressing cells were mixed with ligand-expressing cells and cell-to-cell binding was monitored by detecting the labels associated with the receptor and the ligand. FIG. 1B schematically summarizes the results of the high-throughput cell-cell screening of B7-1 and p75NTR against 395 members of the human immunoglobin (Ig) and TNFR superfamilies. B7-1 expressing cells bound to cells expressing CD28, CTLA-4, and p75NTR, but B7-1 did not bind to cells expressing PD-L1. p75NTR-expressing cells bound to cells expressing B7-1 and PTPRF. FIG. 1C graphically illustrates validation of the binding between B7-1 and p75NTR by recombinant protein titrations using p75NTR-mIgG2A and CTLA-4-mIgG2A to observe biding to streptavidin beads coated with B7-1-hIgG1-biotin. The X-axis shows the concentration of B7-1-hIgG1-biotin in the different assay mixtures. Binding was detected using an anti-mIgG2A ab and flow cytometry. The binding curves were fit and the Bmax, EC50, and hill coefficient were calculated using the equation Y=Bmax*Xh/(Kdh+Xh). FIG. 1D shows that B7-1:p75NTR interactions are conserved in primates, but not mice. B7-1-mCherry expressing cells from various mammalian species were screened against human p75NTR-GFP expressing cells (top left), mouse p75NTR-GFP expressing cells (top right), rat p75NTR-GFP expressing cells (bottom left), or cells expressing only GFP (bottom right). In addition to interacting with cells expressing human p75NTR, cells expressing human B7-1 interacted with cells expressing mouse or rat p75NTR (upper right graph). However, cells expressing mouse B7-1 or rat B7-1 did not interact with p75NTR from any species. p<0.0001 using one way ANOVA with multiple comparisons, n=3-6.

FIG. 2A-2F illustrate which B7-1 amino acids are involved in binding p75NTR. FIG. 2A is a schematic diagram of the B7-1 structure with the B7-1 residues. When the following B7-1 residues are mutated, greater than 25% reduction in B7-1 binding to CD28 and CTLA-4 was observed: I36A, I36D, T39A, K40D, E41A, K43D, V45D, S49A, R63D, Y65A, E69A, K120D, K127D, L131D, and K139D. These amino acids were mapped on to the crystal structure of human B7-1 (PDB: 1I8L). FIG. 2B shows a schematic diagram of the B7-1 structure. The following B7-1 residues exhibited greater than 25% reduction in binding to p75NTR when these B7-1 residues were mutated: I36D, T39A, Y40D, K43D, S49A, R63D, Y65A, N82E, K120D, Y121D, K127D, and K139D. These amino acids were mapped on to the crystal structure of human B7-1 (PDB: 1I8L). FIG. 2C shows a schematic diagram of the B7-1 structure with the same B7-1 residues identified as shown in FIG. 2B, except with a monomer of CTLA-4 also shown. FIG. 2D graphically illustrates that CD28-hIgG1 competes for binding to B7-1 with NGFR. The concentration of concentration of CD28-Fc is shown along the X-axis. FIG. 2E graphically illustrates that CTLA-4-mIgG2A competes for binding to B7-1 with p75NTR, n=3-6. The concentration of concentration of CTLA-4-Fc is shown along the X-axis. FIG. 2F is a schematic diagram illustrating how cells expressing CTLA-4, CD28 or p75 GFP were titrated with recombinant wild type B7-1 or with recombinant mutant B7-1 N82E.

FIG. 3A-3E illustrate the binding interface of B7-1 and PTPRF on P75NTR which was identified using epitope mapping and ligand competition experiments. FIG. 3A shows p75NTR residues that were identified to be important for B7-1 binding. These residues were mapped on to crystal structure of rat p75NTR bound to neurotrophin-3 (NT3) (PDB: 3BUK). Eight point mutations in the p75^{NTR} protein with SEQ ID NO:8 exhibited significant reduction in binding of the p75_{NTR} protein to B7-1. These eight p75_{NTR} amino acids are at positions F136, S137, S137, E147, P150, P150, L165, and R182. Mutations at positions M95, D162, E171, or D104 of the p75_{NTR} protein can reduce or eliminate hydrogen or electrostatic bonds with NGF, proNGF, or NT3, while maintaining binding to B7-1. FIG. 3B illustrates the B7-1 binding site modeled onto the dimeric structure of p75NTR bound to an NT3 dimer (PDB: 3BUK). FIG. 3C illustrates that NGF and B7-1 compete for binding to p75NTR at higher NGF concentrations as determined from ligand competition experiments. FIG. 3D graphically illustrates the percent binding of cells expressing p75NTR with histidine (H) or lysine (K) point replacement mutations at residue F136 (F136K or F136H p75 mutants), to cells expressing the following B7-1 mutants: R63D, N82E, K120D, K127D, and K139D. As illustrated, while most B7-1 mutants reduced binding to p75, the p75NTR-F136K and p75NTR-F136H mutant proteins regained binding comparable to wild type when binding to B7-1N82E expressing cells, p<0.0001 by one way ANOVA with multiple comparisons, n=3-4. FIG. 3E shows structures of B7-1 and p75NTR, indicating proposed interaction between p75NTR-F136 and B7-1-N82.

FIG. 4A-4C illustrate that recombinant B7-1-Fc induces synapse remodeling similarly to proNGF. FIG. 4A shows representative immunofluorescence images of wild type hippocampal neurons treated with proNGF (10 nM), B7-1-Fc (750 nM), or B7-2-Fc (750 nM) and then stained for actin, PSD95, and MAP2. As shown, recombinant B7-1-Fc but not B7-2-Fc affects PSD95 density in cultured neutrons in a manner that is similar to proNGF treatment. FIG. 4B graphically illustrates quantified PSD95 puncta density (PSD95 puncta/μm) for the neurons treated as described of FIG. 4A. As shown, proNGF and B7-1-Fc treated neurons showed significant decreases in PSD95 density compared to control, but B7-2 treated neurons did not exhibit changes in puncta density (p<0.01 by one-way ANOVA with multiple comparisons, n=3 distinct experiments in which 12 neurons were analyzed per experiment). FIG. 4C shows images of DIV18 hippocampal mouse neurons illustrating p75NTR expression as detected by immunofluorescence, scale bar=20 μm.

FIG. 5A-5C illustrate that neurons co-cultured with HEK293 cells expressing B7-1 exhibit significant differences in PSD95 and MAP2 morphology compared to neurons co-cultured with HEK293 cells expressing B7-2 or the B7-1 N82E mutant. FIG. 5A shows representative immune fluorescence images of hippocampal neurons co-cultured with HEK293 cells that express B7-1, B7-2, or the B7-1 N82E mutant. FIG. 5B graphically illustrates quantitative differences in PSD95 density in dendrites that were in direct contact with HEK293 cells that express B7-1, B7-2, or B7-1 N82E. FIG. 5C graphically illustrates quantitative differences in MAP2 morphology in dendrites that were in direct contact with HEK293 cells that express B7-1, B7-2, or B7-1 N82E. n=3 independent experiments in which 12-14 individual neurons were analyzed per treatment per experiment. One-way ANOVA, Tukey's multiple comparisons test p<0.001 (***), n=3, scale bar=20 μm.

FIG. 6A-6B illustrates increased expression of B7-1 and/or p75NTR in from a mouse model of Alzheimer's Disease. FIG. 6A shows sections of brains from a mouse model of Alzheimer's Disease (CRND8 transgenic mice, C8) exhibiting increased expression of B7-1 and increased expression of the activated microglia marker Iba1 compared to brains from wild type (WT) mice. FIG. 6B shows sections of brains from a mouse model of Alzheimer's Disease (CRND8 transgenic mice) exhibiting increased expression of p75NTR compared to wild type mouse brains at 7 months. Goat anti-p75NTR was used to detect p75NTR. The top six frames are images at 20× magnification, while the bottom six frames are a 40× magnification.

FIG. 7A-7D show that introduction of B7-1 into mouse brains (adult subiculum, involved in memory retrieval and spatial encoding) induces loss of synapses. FIG. 7A shows images of Golgi-stained pyramidal neurons within wild type mouse brains 3 hours after in vivo injection of hB7-1-Fc (500 ng/μL), hB7-2-Fc (500 ng/μL) or saline into the subiculum region of hippocampal formation at P75. As illustrated, injection of B7-1 induces the pruning of dendritic spines in vivo (central images). Such effects on dendritic processes were not observed when saline or B7-2 was injected into the subiculum of mice (left and right images respectively). FIG. 7B graphically illustrates number of spines per μm at various distances from the associated neuronal cell bodies in representative brain sections of mice injected with either B7-1 or B72. There was a significant reduction in total spine density at 3 hours after hB7-1 injection, compared to injection of saline or B7-2. The apical dendrite segment 50-150 μm away from cell soma was chosen for quantification. FIG. 7C shows images of Golgi-stained pyramidal neurons within the brains of p75(−/−) mice 3 hours after in vivo injection of hB7-1-Fc (500 ng/μL), hB7-2-Fc (500 ng/μL) or saline into the subiculum region of hippocampal formation at P75. As illustrated, no significant changes were observed when B7-1 was injected into the subiculum region when the neurons lacked p75 neurotrophin receptor. FIG. 7D graphically illustrates the number of spines per μm at various distances from the associated neuronal cell bodies in representative brain sections of p75(−/−) mice 3 hours after in vivo injection of hB7-1-Fc (500 ng/μL), hB7-2-Fc (500 ng/μL) or saline. As illustrated, there was no significant changes in total spine density 3 hours after hB7-1 or hB7-2 was injected compared to injection of saline. The apical dendrite segment 50-150 μm away from cell soma was chosen for quantification. One-way ANOVA, post hoc Tukey's test. Scale bar: 20 μm. n=4 brains/treatment (20 neurons/brain). (*) p=0.0268; data are represented as mean±SEM). n.s.: not significant.

FIG. 8A-8F show that ORENCIA® (abatacept) blocks B7-1 induced synaptic loss. FIG. 8A shows that B7-1 induced synaptic spine loss and MAP2 fragmentation in dendrites is inhibited by ORENCIA® (abatacept) in neuronal cultures from wildtype mice. B7-2 did not induce spine loss or MAP2 fragmentation. FIG. 8B shows that in neuronal cultures from p75 null mutant mice, B7-1 failed to induce MAP2 fragmentation, and Orencia™ had no effect. FIG. 8C graphically illustrates that abatacept blocks B7-1 binding to p75. Cells expressing p75 were incubated with soluble B7-1 in the presence or absence of abatacept. As illustrated, B7-1 binding to p75 was reduced at least 4-fold or more when abatacept was present. FIG. 8D shows images of wild type hippocampal neurons co-cultured with HEK 293-hB7-1, HEK 293-hB7-1 N82E, HEK 293-hB7-2, untreated or treated with abatacept. FIG. 8E graphically illustrates the MAP2 continuity scores of dendrites in direct contact with different HEK 293-hB7-1, HEK 293-hB7-1 N82E, HEK 293-hB7-2 cell lines that were either untreated or treated with abatacept. FIG. 8F graphically illustrates the MAP2 continuity scores of p75^{-/-} neurons in direct contact with different with different HEK 293-hB7-1, HEK 293-hB7-1 N82E, HEK 293-hB7-2 cell lines that were either untreated or treated with abatacept. (p<0.01 by two-way ANOVA, n=3). For MAP2 continuity, the MAP2 channel was captured with ImageJ software and a threshold applied. The plot profile tool quantified thresholded MAP2 signal along the dendrite. The area under the curve of each linear path was then divided by 255*path length and multiplied by 100 to determine % continuity of MAP2 signal on the dendrite.

FIG. 9A-9D illustrate generating a chimeric mouse line that expresses human B7-1. FIG. 9A shows a schematic diagram of the exons and numbers of amino acids in human and mouse B7-1 genes. A full IgV domain exon swapping strategy was used to make B7-1 human-mouse chimera protein. The arrow identifies the swapped exons. FIG. 9B graphically illustrates the percent of cells expressing human B7-1-GFP (left) or human-mouse chimera B7-1-GFP (right) that were bound to increasing concentrations of mouse CTLA-4-mIgG2a. FIG. 9C graphically illustrates the percent of cells expressing human B7-1-GFP (left) or human-mouse chimera B7-1-GFP (right) that were bound to increasing concentrations of recombinant p75 mIgG2a. Binding was assessed using an anti mIgG2a-647 labeled antibody and flow cytometry. FIG. 9D is a western blot illustrating that the chimera mice expressed B7-1 human-mouse chimera protein in their spleens.

FIG. 10A-10C illustrate that splenocyte populations from chimera mice expressed B7-1 human-mouse chimera protein (h:mB7-1 KI mice) upregulate B7-1 expression in response to activation stimuli. FIG. 10A illustrates flow cytometry results of splenocytes from WT and h:mB7-1 knock-in animals (2×10⁶ cells) that were incubated for 3 days with either 1) untreated (\\\hatching) or 2) LPS (///hatching; 10 ug/mL) or 3) plated in wells coated with anti-mCD3 (1 ug/mL overnight) and soluble anti-mCD28 (5 ug/mL) (\\\hatching). Cells were then stained with antibodies against mCD45, mCD3, mCD11b, mB7-1 or hB7-1 followed by DAPI staining. Cells were washed and analyzed by flow cytometry, gating for DAPI negative and CD45 positive cells. CD3(+) and CD3(−) populations were then sub-gated and CD11b(+) was further sub-gated from the CD3(−) population. The CD3(−)CD11b(+) and CD3(+) populations were then gated for mB7-1 expression (WT animals) or hB7-1 expression (KI animals), yielding the results in FIG. 10B-10C. FIG. 10B graphically illustrates the percentage of cells positive for B7-1 expression in the sub-gated population of CD3(−)CD11b(+) cells from flow data presented in FIG. 10A. Average of N=2 animals. FIG. 10C graphically illustrates the percentage of cells positive for B7-1 expression in the sub-gated population of CD3+ cells from flow data presented in FIG. 10A. Average of N=2 animals.

### DETAILED DESCRIPTION

As described herein the immunomodulatory molecule B7-1 (CD80) interacts with the p75NTR, a member of the TNF receptor superfamily. The locations of the binding surfaces of B7-1 and p75NTR as well as the specific amino acids required for binding between B7-1 and p75NTR were defined as described herein. During neurodegenerative conditions p75NTR is upregulated in neurons, and B7-1 is upregulated in microglia. For example, addition of B7-1 (soluble or bound to cells) to cultured neurons acutely disassembles synapses in a p75NTR dependent manner, and delivery of B7-1 acutely induces synaptic loss in the subiculum of mice as assessed by Golgi analysis. The addition of a reagent that binds to B7-1 (CTLA-4-Fc, Orencia™) blocks such synaptic elimination in cultured hippocampal neurons.

Hence, methods of reducing synaptic loss are described herein that involve administration of one or more reagents that bind to B7-1 (CTLA-4-Fc, Orencia™) or that bind to p75 NTR to a subject. Such a subject may be in need thereof.

The results on B7-1:p75NTR interactions illustrated herein also indicate that a variety of agents can be used. Methods are described herein for selecting new therapeutic strategies for a variety of neuronal and nervous system conditions and disorders.

For example, neuronal and nervous system conditions and disorders that can be treated using the compositions and methods described herein include neuronal injuries, neuronal inflammation, acute traumatic injuries to the nervous system, Alzheimer's disease, CNS disorders, cerebral ischemia, lupus, Parkinson's disease, multiple sclerosis, stroke, spinal cord injury, psychological problems, and other neuronal conditions.

The prevailing understanding has been that p75, a TNFR superfamily member, engages with neurotrophic factors to mediate its various biological functions. However, as described and shown herein B7-1 is a ligand that is on immune cells, and p75 on neuronal cells interacts with such immune cell-bound B7-1 to elicit previously unappreciated contributions to neuronal synapse function. Notably, this interaction represents an unusual example of functional cross-talk between the immunoglobulin and TNF-receptor superfamilies. This observation also expands the repertoire of actions that immune cells can perform to modulate the nervous system. Moreover, the experimental results described herein show that blockade of the B7-1:p75 interaction represents a viable strategy for ameliorating synaptic defects observed in neurodegenerative conditions.

### B7-1 (CD80) Protein

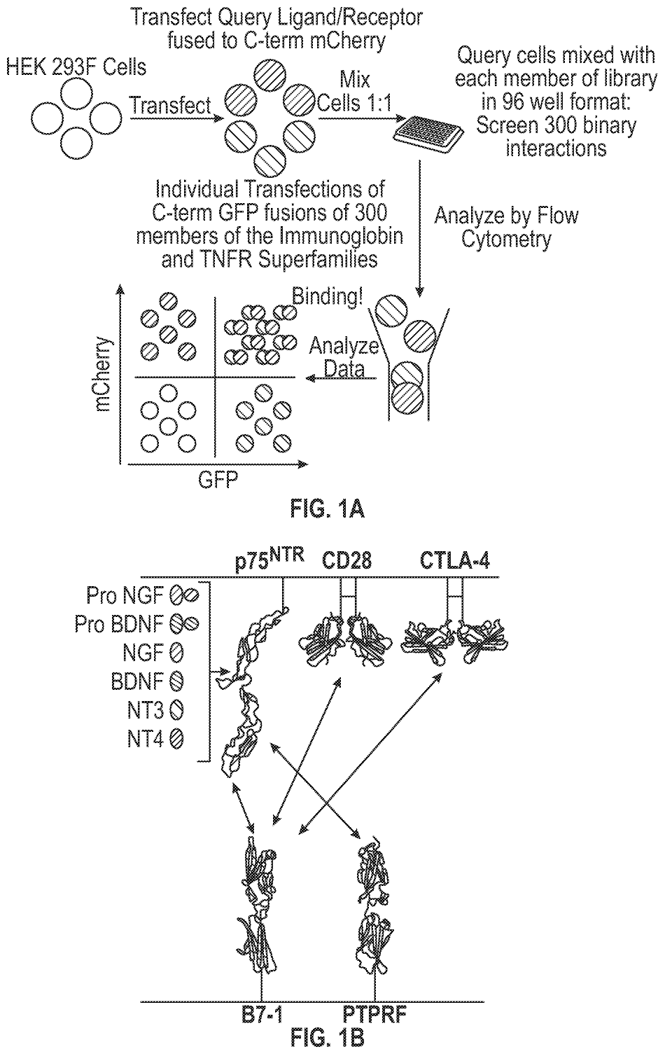

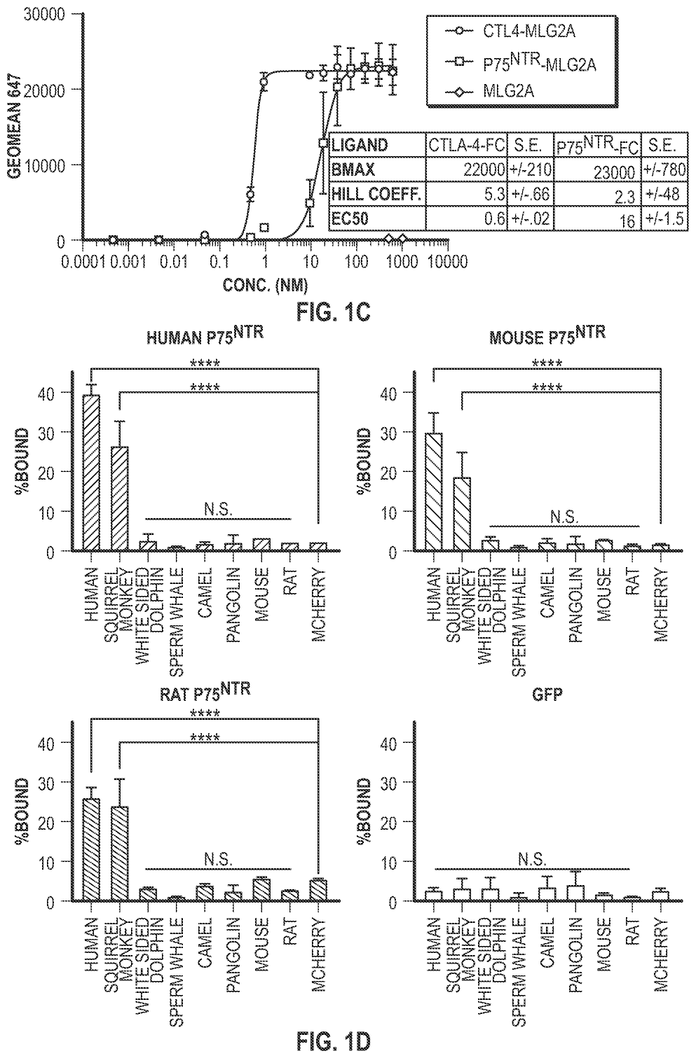

B7-1 (also called T-lymphocyte activation antigen CD80 precursor) is an immunomodulatory ligand that has an extracellular membrane distal IgV and membrane proximal IgC domains. B7-1 is therefore a transmembrane protein and a member of the immunoglobulin superfamily (IgSF). B7-1 can be expressed by dendritic cells, macrophages, microglia and B cells, and it can regulate T cell function in the context of antigen presentation through interactions with CD28 and CTLA-4 (in trans) and PD-L1 (in cis). B7-1, and the related B7-2 ligand, co-stimulate T cells through interactions with CD28, and co-inhibit T cells through interaction with CTLA-4 (see, e.g., FIG. 1B). However, while most antigen presenting cells express the B7-2 ligand under basal conditions, they do not express significant levels of B7-1 until they become activated by inflammatory pathways in the setting of infection, injury or aging.

Upregulation of B7-1 by antigen presenting cells is observed in response to a broad array of inflammatory conditions, specifically following IFNγ exposure, activation by Toll-like receptors, and by infiltrating T helper cells. B7-1 is also upregulated in rodent models of experimental autoimmune encephalitis, stroke, and traumatic brain injury.

In humans, B7-1 is upregulated in microglia of multiple sclerosis patients, is upregulated by human microglia in response to LPS or IFNγ and is expressed by B cells and monocytes in the cerebrospinal fluid of patients with multiple sclerosis or nervous system infection. While evaluation of B7-1 expression in other conditions has not been systematically examined, induction of peripheral expression of B7-1 by monocytes has been observed with aging (Busse et al. J Alzheimers Dis 47, 177-184 (2015)) and in patients with mild cognitive impairment (Famenini et al. FASEB J 31, 148-160 (2017)).

B7-1 can modulate T-cell function through interactions with CD28, CTLA-4, and PD-L1, and is typically expressed at low levels in the central nervous systems.

As demonstrated herein B7-1 is upregulated by microglia in neurodegenerative diseases, as well as in mouse lines used as models for Alzheimer's Disease research. B7-1 expression is upregulated on activated microglia and brain infiltrating macrophages in response to a number of cues, including exposure to LPS and proinflammatory cytokines, traumatic brain injury, infections of the brain, and neurodegenerative diseases such as Multiple Sclerosis and Alzheimer's Disease. Hence, as described herein, inhibition of B7-1 function can reduce the onset and progression of a variety of neurological conditions and diseases.

A sequence for a Homo sapiens B7-1 protein is shown below as SEQ ID NO:1.

| | |
|---|---|
| 1 | MGHTRRQGTS PSKCPYLNFF QLLVLAGLSH FCSGVIHVTY |
| 41 | EVKEVATLSC GHNVSVEELA QTRIYWQKEK KMVLTMMSGD |
| 81 | MNIWPEYKNR TIFDITNNLS IVILALRPSD EGTYECVVLK |
| 121 | YEKDAFKREH LAEVTLSVKA DFPTPSISDF EIPTSNIRRI |
| 161 | ICSTSGGFPE PHLSWLENGE ELNAINTTVS QDPETELYAV |
| 201 | SSKLDFNMTT NHSFMCLIKY GHLRVNQTFN WNTTKQEHFP |
| 241 | DNLLPSWAIT LISVNGIFVI CCLTYCFAPR CRERRRNERL |
| 281 | RRESVRPV |

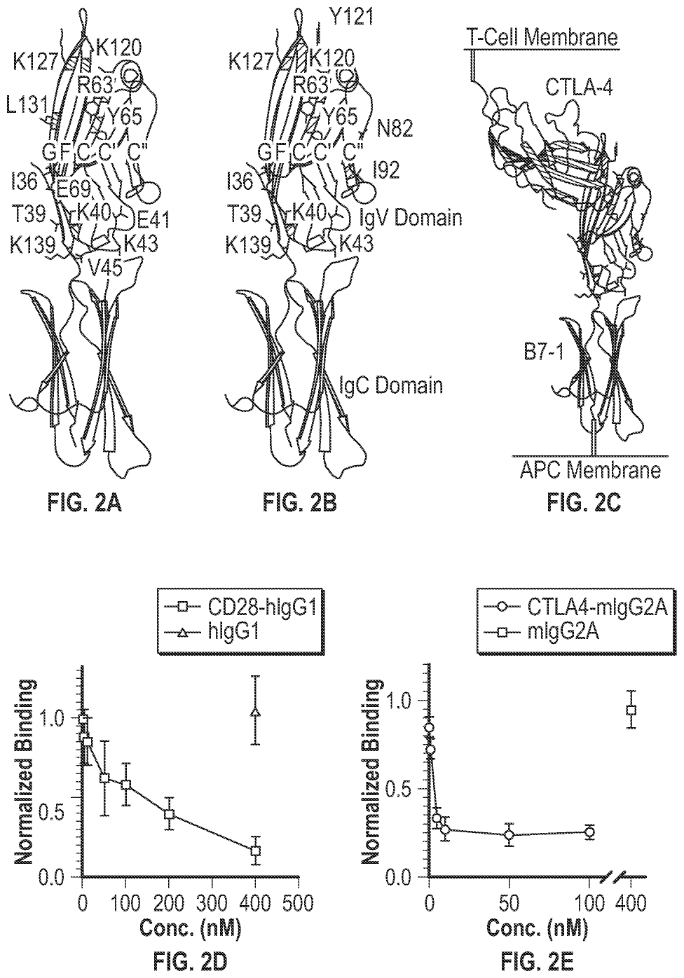

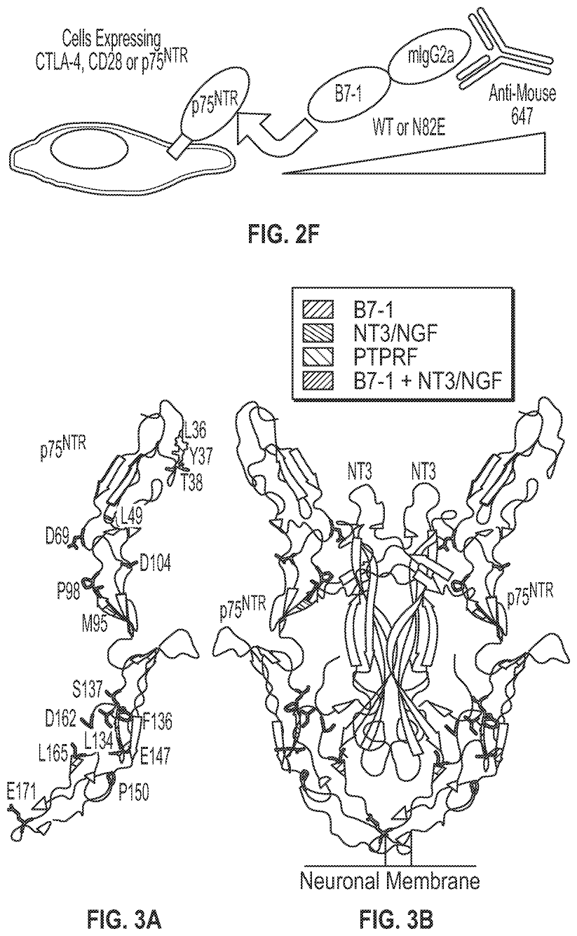

Data described herein shows that B7-1 binds to p75 neurotrophin receptor protein via amino acids I36, T39, Y40, K43, S49, R63, Y65, N82, K120, Y121, K127, and K139. These amino acid positions are highlighted in bold and with underlining for the SEQ ID NO:1 sequence shown above. The following mutations in the B7-1 protein decreased B7-1 binding to p75NTR by more than 25%: I36D, T39A, Y40D, K43D, S49A, R63D, Y65A, N82E, K120D, Y121D, K127D, and K139D (FIG. 2B). Of these, N82E, I92D, and Y121D specifically cause losses in binding to p75NTR, and the N82E, I92D, and Y121D mutations do not affect interaction with CTLA-4, CD28, or PD-L1. In particular, mutation of the N82 residue substantially eliminates binds of B7-1 binding to p75NTR.

The Homo sapiens B7-1 protein also has a transmembrane domain at about amino acid positions 243-263 (LLPSWAIT LISVNGIFVI CCL; SEQ ID NO:2). Removal or mutation of this transmembrane domain can facilitate preparation of a soluble form of BF-1. As illustrated herein, soluble B7-1 can induce acute synaptic elimination via its interaction with p75NTR. This B7-1 transmembrane domain can in some cases be modified to provide a modified B7-1 protein that does not remain bound to cells. Such a modified B7-1 protein can be used to facilitate analysis of B7-1: p75NTR interactions and the biological effects of such interactions. Modification of the B7-1 transmembrane domain can include replacement or deletion of amino acids in the B7-1 transmembrane domain. In some cases, modification of the B7-1 transmembrane domain can include insertion of amino acids that have chemical and physical properties that are different from the amino acids in the wild type B7-1 transmembrane domain.

A related sequence for a Homo sapiens B7-1 protein having NCBI accession no. NP_005182.1 is shown below as SEQ ID NO:3, where the Y40 residue is lysine (K) at position 40.

| | |
|---|---|
| 1 | MGHTRRQGTS PSKCPYLNFF QLLVLAGLSH FCSGVIHVTK |
| 41 | EVKEVATLSC GHNVSVEELA QTRIYWQKEK KMVLTMMSGD |
| 81 | MNIWPEYKNR TIFDITNNLS IVILALRPSD EGTYECVVLK |
| 121 | YEKDAFKREH LAEVTLSVKA DFPTPSISDF EIPTSNIRRI |
| 161 | ICSTSGGFPE PHLSWLENGE ELNAINTTVS QDPETELYAV |
| 201 | SSKLDFNMTT NHSFMCLIKY GHLRVNQTFN WNTTKQEHFP |
| 241 | DNLLPSWAIT LISVNGIFVI CCLTYCFAPR CRERRRNERL |
| 281 | RRESVRPV |

Nucleic acid segments encoding wild type or modified forms of human and chimeric B7-1 proteins can be operably linked to a promoter to thereby generate an expression cassette useful for expressing the wild type or modified forms of human and chimeric B7-1 proteins.

A cDNA sequence for the Homo sapiens B7-1 protein with SEQ ID NO:3 is shown below as SEQ ID NO:4 (accorded NCBI accession no. NM_005191.4).

| | |
|---|---|
| 1 | AAACCCTCTG TAAAGTAACA GAAGTTAGAA GGGGAAATGT |
| 41 | CGCCTCTCTG AAGATTACCC AAAGAAAAAG TGATTTGTCA |
| 81 | TTGCTTTATA GACTGTAAGA AGAGAACATC TCAGAAGTGG |
| 121 | AGTCTTACCC TGAAATCAAA GGATTTAAAG AAAAAGTGGA |
| 161 | ATTTTTCTTC AGCAAGCTGT GAAACTAAAT CCACAACCTT |
| 201 | TGGAGACCCA GGAACACCCT CCAATCTCTG TGTGTTTTGT |
| 241 | AAACATCACT GGAGGGTCTT CTACGTGAGC AATTGGATTG |
| 281 | TCATCAGCCC TGCCTGTTTT GCACCTGGGA AGTGCCCTGG |
| 321 | TCTTACTTGG GTCCAAATTG TTGGCTTTCA CTTTTGACCC |
| 361 | TAAGCATCTG AAGCCATGGG CCACACACGG AGGCAGGGAA |
| 401 | CATCACCATC CAAGTGTCCA TACCTCAATT TCTTTCAGCT |
| 441 | CTTGGTGCTG GCTGGTCTTT CTCACTTCTG TTCAGGTGTT |
| 481 | ATCCACGTGA CCAAGGAAGT GAAAGAAGTG GCAACGCTGT |
| 521 | CCTGTGGTCA CAATGTTTCT GTTGAAGAGC TGGCACAAAC |
| 561 | TCGCATCTAC TGGCAAAAGG AGAAGAAAAT GGTGCTGACT |
| 601 | ATGATGTCTG GGGACATGAA TATATGGCCC GAGTACAAGA |
| 641 | ACCGGACCAT CTTTGATATC ACTAATAACC TCTCCATTGT |
| 681 | GATCCTGGCT CTGCGCCCAT CTGACGAGGG CACATACGAG |
| 721 | TGTGTTGTTC TGAAGTATGA AAAAGACGCT TTCAAGCGGG |
| 761 | AACACCTGGC TGAAGTGACG TTATCAGTCA AAGCTGACTT |
| 801 | CCCTACACCT AGTATATCTG ACTTTGAAAT TCCAACTTCT |
| 841 | AATATTAGAA GGATAATTTG CTCAACCTCT GGAGGITTTC |
| 881 | CAGAGCCTCA CCTCTCCTGG TTGGAAAATG GAGAAGAATT |
| 921 | AAATGCCATC AACACAACAG TTTCCCAAGA TCCTGAAACT |
| 961 | GAGCTCTATG CTGTTAGCAG CAAACTGGAT TTCAATATGA |
| 1001 | CAACCAACCA CAGCTTCATG TGTCTCATCA AGTATGGACA |
| 1041 | TTTAAGAGTG AATCAGACCT TCAACTGGAA TACAACCAAG |
| 1081 | CAAGAGCATT TTCCTGATAA CCTGCTCCCA TCCTGGGCCA |
| 1121 | TTACCTTAAT CTCAGTAAAT GGAATTTTTG TGATATGCTG |
| 1161 | CCTGACCTAC TGCTTTGCCC CAAGATGCAG AGAGAGAAGG |
| 1201 | AGGAATGAGA GATTGAGAAG GGAAAGTGTA CGCCCTGTAT |
| 1241 | AACAGTGTCC GCAGAAGCAA GGGGCTGAAA AGATCTGAAG |
| 1281 | GTCCCACCTC CATTTGCAAT TGACCTCTTC TGGGAACTTC |
| 1321 | CTCAGATGGA CAAGATTACC CCACCTTGCC CTTTACGTAT |
| 1361 | CTGCTCTTAG GTGCTTCTTC ACTTCAGTTG CTTTGCAGGA |
| 1401 | AGTGTCTAGA GGAATATGGT GGGCACAGAA GTAGCTCTGG |
| 1441 | TGACCTTGAT CAAGGTGTTT TGAAATGCAG AATTCTTGAG |
| 1481 | TTCTGGAAGG GACTTTAGAG AATACCAGTG TTATTAATGA |
| 1521 | CAAAGGCACT GAGGCCCAGG GAGGTGACCC GAATTATAAA |
| 1561 | GGCCAGCGCC AGAACCCAGA TTTCCTAACT CTGGTGCTCT |
| 1601 | TTCCCTTTAT CAGTTTGACT GTGGCCTGTT AACTGGTATA |
| 1641 | TACATATATA TGTCAGGCAA AGTGCTGCTG GAAGTAGAAT |
| 1681 | TTGTCCAATA ACAGGTCAAC TTCAGAGACT ATCTGATTTC |
| 1721 | CTAATGTCAG AGTAGAAGAT TTTATGCTGC TGTTTACAAA |
| 1761 | AGCCCAATGT AATGCATAGG AAGTATGGCA TGAACATCTT |
| 1801 | TAGGAGACTA ATGGAAATAT TATTGGTGTT TACCCAGTAT |
| 1841 | TCCATTTTTT TCATTGTGTT CTCTATTGCT GCTCTCTCAC |
| 1881 | TCCCCCATGA GGTACAGCAG AAAGGAGAAC TATCCAAAAC |
| 1921 | TAATTTCCTC TGACATGTAA GACGAATGAT TTAGGTACGT |
| 1961 | CAAAGCAGTA GTCAAGGAGG AAAGGGATAG TCCAAAGACT |
| 2001 | TAACTGGTTC ATATTGGACT GATAATCTCT TTAAATGGCT |
| 2041 | TTATGCTAGT TTGACCTCAT TTGTAAAATA TTTATGAGAA |
| 2081 | AGTTCTCATT TAAAATGAGA TCGTIGTTTA CAGTGTATGT |
| 2121 | ACTAAGCAGT AAGCTATCTT CAAATGTCTA AGGTAGTAAC |
| 2161 | TTTCCATAGG GCCTCCTTAG ATCCCTAAGA TGGCTTTTTC |
| 2201 | TCCTTGGTAT TTCTGGGTCT TTCTGACATC AGCAGAGAAC |
| 2241 | TGGAAAGACA TAGCCAACTG CTGTTCATGT TACTCATGAC |
| 2281 | TCCTTTCTCT AAAACTGCCT TCCACAATTC ACTAGACCAG |
| 2321 | AAGTGGACGC AACTTAAGCT GGGATAATCA CATTATCATC |
| 2361 | TGAAAATCTG GAGTTGAACA GCAAAAGAAG ACAACATTTC |
| 2401 | TCAAATGCAC ATCTCATGGC AGCTAAGCCA CATGGCTGGG |
| 2441 | ATTTAAAGCC TTTAGAGCCA GCCCATGGCT TTAGCTACCT |
| 2481 | CACTATGCTG CTTCACAAAC CTTGCTCCTG TGTAAAACTA |
| 2521 | TATTCTCAGT GTAGGGCAGA GAGGTCTAAC ACCAACATAA |
| 2561 | GGTACTAGCA GTGTTTCCCG TATTGACAGG AATACTTAAC |
| 2601 | TCAATAATTC TTTTCTTTTC CATTTAGTAA CAGTTGTGAT |
| 2641 | GACTATGTTT CTATTCTAAG TAATTCCTGT ATTCTACAGC |
| 2681 | AGATACTTTG TCAGCAATAC TAAGGGAAGA AACAAAGTTG |
| 2721 | AACCGTTTCT TTAATAA |

A sequence for a mouse B7-1 protein is shown below (NCBI NP_001346827.1; SEQ ID NO:5).

| | |
|---|---|
| 1 | MACNCQLMQD TPLLKFPCPR LILLFVLLIR LSQVSSDVDE |
| 41 | QLSKSVKDKV LLPCRYNSPH EDESEDRIYW QKHDKVVLSV |
| 81 | IAGKLKVWPE YKNRTLYDNT TYSLIILGLV LSDRGTYSCV |
| 121 | VQKKERGTYE VKHLALVKLS IKADFSTPNI TESGNPSADT |
| 161 | KRITCFASGG FPKPRFSWLE NGRELPGINT TISQDPESEL |
| 201 | YTISSQLDFN TTRNHTIKCL IKYGDAHVSE DFTWEKPPED |
| 241 | PPDSKNTLVL FGAGFGAVIT VVVIVVIIKC FCKHRSCFRR |
| 281 | NEASRETNNS LTFGPEEALA EQTVFL |

A cDNA sequence for the SEQ ID NO:5 mouse B7-1 protein is shown below as SEQ ID NO:6 (NCBI NM_001359898.1).

| | |
|---|---|
| 1 | GAGTTTTATA CCTCAATAGA CTCTTACTAG TTTCTCTTTT |
| 41 | TCAGGTTGTG AAACTCAACC TTCAAAGACA CTCTGTTCCA |
| 81 | TTTCTGTGGA CTAATAGGAT CATCTTTAGC ATCTGCCGGG |
| 121 | TGGATGCCAT CCAGGCTTCT TTTTCTACAT CTCTGTTTCT |
| 161 | CGATTTTTGT GAGCCTAGGA GGTGCCTAAG CTCCATTGGC |
| 201 | TCTAGATTCC TGGCTTTCCC CATCATGTTC TCCAAAGCAT |
| 241 | CTGAAGCTAT GGCTTGCAAT TGTCAGTTGA TGCAGGATAC |
| 281 | ACCACTCCTC AAGTTTCCAT GTCCAAGGCT CATTCTTCTC |
| 321 | TTTGTGCTGC TGATTCGTCT TTCACAAGTG TCTTCAGATG |
| 361 | TTGATGAACA ACTGTCCAAG TCAGTGAAAG ATAAGGTATT |
| 401 | GCTGCCTTGC CGTTACAACT CTCCTCATGA AGATGAGTCT |
| 441 | GAAGACCGAA TCTACTGGCA AAAACATGAC AAAGTGGTGC |
| 481 | TGTCTGTCAT TGCTGGGAAA CTAAAAGTGT GGCCCGAGTA |
| 521 | TAAGAACCGG ACTTTATATG ACAACACTAC CTACTCTCTT |
| 561 | ATCATCCTGG GCCTGGTCCT TTCAGACCGG GGCACATACA |
| 601 | GCTGTGTCGT TCAAAAGAAG GAAAGAGGAA CGTATGAAGT |
| 641 | TAAACACTTG GCTTTAGTAA AGTIGTCCAT CAAAGCTGAC |
| 681 | TTCTCTACCC CCAACATAAC TGAGTCTGGA AACCCATCTG |
| 721 | CAGACACTAA AAGGATTACC TGCTTTGCTT CCGGGGGTTT |
| 761 | CCCAAAGCCT CGCTTCTCTT GGTTGGAAAA TGGAAGAGAA |
| 801 | TTACCTGGCA TCAATACGAC AATTTCCCAG GATCCTGAAT |
| 841 | CTGAATTGTA CACCATTAGT AGCCAACTAG ATTTCAATAC |
| 881 | GACTCGCAAC CACACCATTA AGTGTCTCAT TAAATATGGA |
| 921 | GATGCTCACG TGTCAGAGGA CTTCACCTGG GAAAAACCCC |
| 961 | CAGAAGACCC TCCTGATAGC AAGAACACAC TTGTGCTCTT |
| 1001 | TGGGGCAGGA TTCGGCGCAG TAATAACAGT CGTCGTCATC |
| 1041 | GTTGTCATCA TCAAATGCTT CTGTAAGCAC AGAAGCTGTT |
| 1081 | TCAGAAGAAA TGAGGCAAGC AGAGAAACAA ACAACAGCCT |
| 1121 | TACCTTCGGG CCTGAAGAAG CATTAGCTGA ACAGACCGTC |
| 1161 | TTCCTTTAGT TCTTCTCTGT CCATGTGGGA TACATGGTAT |
| 1201 | TATGTGGCTC ATGAGGTCTC ATCTACCATT TGCAACTGAC |
| 1241 | CTCTTCTGCA AAGGACTTCA GAAACCTAGC ACTACCCTGG |
| 1281 | CTCTGCAAAC ACGGTTCTCT AGGTGAAGCC TCTGCAGTGG |
| 1321 | TTTGCAGAAG TACTCAGACG AATGAACCAC AGTAGTTCTG |
| 1361 | CTGATTCTGA GGACGTAGTT TAGAGACTGA ATTCTTTGGA |
| 1401 | AAGGACATAG GGACGATTTG CACATTTGCT TGCACATCAC |
| 1441 | ACACACACAC ACACACACAC ACACACACAC ACCCACACAC |
| 1481 | ACACACTCTC TCTCGATACC TTAGGATAGG GTTCTACCCT |
| 1521 | GTTGCTCAGT GACAAAGAAT CACTCTGTGG CGGAGGCAGG |
| 1561 | CTTCAAGCTT GCAGCAATCC TCCTGCACCA GTTTCCTGAG |
| 1601 | TGCCAGACTT CCAGGTGTAA GCTATGGCAC TTAGCAGAAC |
| 1641 | ACTAGCTGAA TCAATGAAGA CACTGAGGTT CCAAGAGGGA |
| 1681 | ACCTGAATTA TGAAGGTGAG TCAGAATCCA GATTTCCTGG |
| 1721 | CTCTACCACT CTTAACCTGT ATCTGTTAGA CCCCAAGCTC |
| 1761 | TGAGCTCATA GACAAGCTAA TTTAAAATGC TTTTAATAAG |
| 1801 | CAGAAGGCTC AGTTAGTACG GGGTTCAGGA TACTGCTTAC |
| 1841 | TGGCAATATT TGACTAGCCT CTATTTTGTT TGTTTTTTAA |
| 1881 | GGCCTACTGA CTGTAGTGTA ATTTGTAGGA AACATGTTGC |
| 1921 | TATGTATACC CATTTGAGGG TAATAAAAAT GTTGGTAATT |
| 1961 | TTCAGCCAGC ACTTTCCAGG TATTTCCCTT TTTATCCTTC |
| 2001 | ATGTCCTTAA AAGAAACCAG AGTCAACCAG AATTAACTGC |
| 2041 | TTTTGTTGTC TAAGATGAAT GTATGCCTTT TATACTTCAA |
| 2081 | AGCAAAAGTC AAGTTGGAGG CACAGTTCGT AACTTACTCG |
| 2121 | TTCATATTGA ACTTATAATG AATGTCTTTA GGCAACTTTG |
| 2161 | CCCTTTTACA CAGTTCTTGA TATAGTTCTT ATTTCAAATG |
| 2201 | AGATTATTTA CTGAGTAATC ATCTGTCCTC ACATAAACAA |
| 2241 | CTTAAGTGAT ACTAGCCTTA CACAGAACTT CCTCAGATTC |
| 2281 | CTGGAAGTGG CTTTTGCTCT TTGGTATAGA TCTGCTACAG |
| 2321 | TGGAATTGAA AAATAAAATG TACGTTAGAC ACACTTGCCT |
| 2361 | GTAATCCCTG CACTCAGTAT ACTGAGGCTG AAGAACTGAA |
| 2401 | TTCCAGGTAG CATGGCCTGG ATAGTAAGAT CCTGTCTGAA |
| 2441 | ACCCACTCCT CCATCAATGT GCCCCAAAGA AATGGAAAAC |
| 2481 | AAGAAGCAGC TAACTGCTGC TTCTATACTG AAGGTTCCCT |
| 2521 | TTTCTCTGAG ATGTAATCCA CAACCTACAA GACCAGCAGT |
| 2561 | GAGCTTAAGT CAAGACAATC AGATATTCTA GTCTGAATTT |
| 2601 | ATAGGAGGAC ATCGAATGGC AGCCAAACTT CTCAACGGGA |
| 2641 | CCAGAATCTG TTGTATGCCC AGGAAACAGG TCCATTTTTA |
| 2681 | TGTGGGTG |

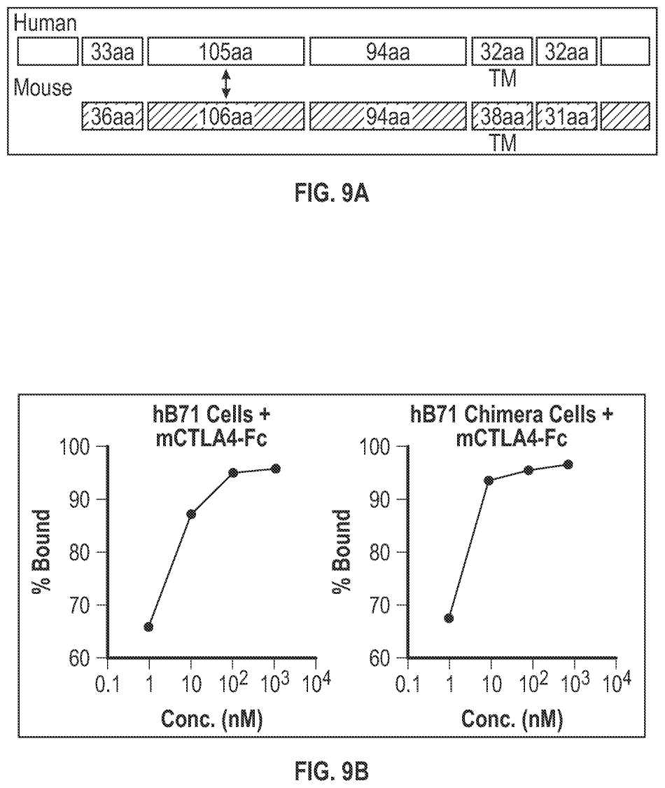

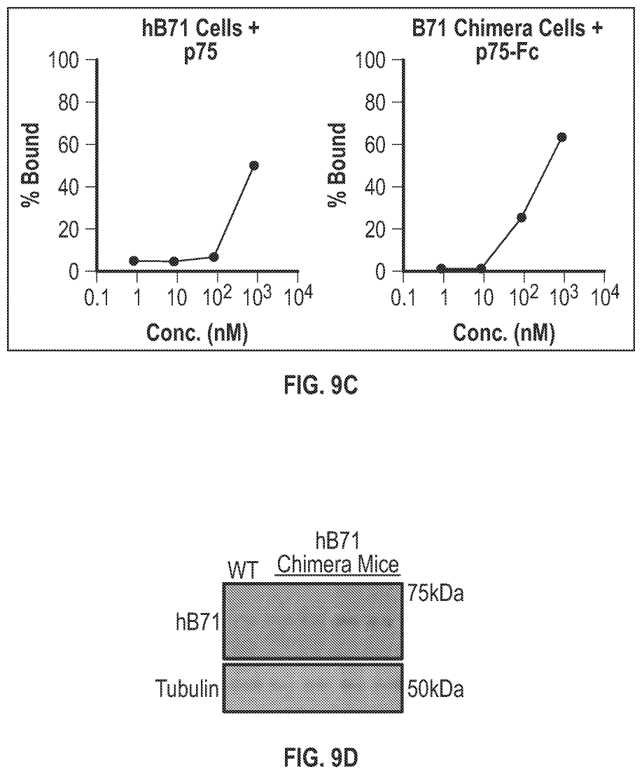

As illustrated herein, a chimeric human-mouse B7-1 protein was made that can be expressed in an animal model to provide human-like B7-1 binding and functional properties in the animal model (e.g., in a mouse). The chimeric B7-1 protein is mostly human B7-1 but it has human B7-1 exon 2 replaced by murine exon 3 (FIG. 9A). Mice embryos were transfected with an expression vector having a promoter operably linked to a cDNA encoding such a chimeric B7-1 protein. As shown herein, the chimeric B7-1 protein was also expressed in HEK 293 cells to evaluate its protein stability and interaction with human and murine p75. FIG. 9C shows that chimeric human-mouse B7-1 protein did bind p75 and CTLA-4. Further experiments showed that the chimeric human-mouse B7-1 protein also bound CD28. A sequence for the human-mouse chimeric protein is shown below as SEQ ID NO:7.

| | |
|---|---|
| 1 | MACNCQLMQD TPLLKFPCPR LILLFVLLIR LSQVSSGVIH |
| 41 | VTKEVKEVAT LSCGHNVSVE ELAQTRIYWQ KEKKMVLTMM |
| 81 | SGDMNIWPEY KNRTIFDITN NLSIVILALR PSDEGTYECV |
| 121 | VLKYEKDAFK REHLAEVTLS VKADFSTPNI TESGNPSADT |
| 161 | KRITCFASGG FPKPRFSWLE NGRELPGINT TISQDPESEL |
| 201 | YTISSQLDFN TTRNHTIKCL IKYGDAHVSE DFTWEKPPED |
| 241 | PPDSKNTLVL FGAGFGAVIT VVVIVVIIKC FCKHRSCFRR |
| 281 | NEASRETNNS LTFGPEEALA EQTVFL |

Variants and homologs of B7-1 can be present in various subject. Hence, the B7-1 protein can have a sequence with at least 70%, or at least 75%, or at least 80%, or at least 85%, or at least 90%, or at least 85%, or at least 90%, or at least 95%, or at least 96%, or at least 97%, or at least 98%, or at least 99% sequence identity to any of B7-1 sequences described herein.

### p75 Neurotrophin Receptor

The p75 neurotrophin receptor (p75^{NTR}, NGFR, TNFRSF16, CD271) is a member of the family of transmembrane receptors for the tumor necrosis factor family of cytokines. Each receptor of this family shares a common extracellular structure that includes conserved cysteine-rich repeats. The NGF, BDNF, NT-3, and NT-4/5 factors bind to the p75 neurotrophin receptor. The p75 neurotrophin receptor is upregulated on neurons following acute injury, or in neurodegenerative conditions. As described herein, B7-1 also binds to the p75 neurotrophin receptor, and such B7-1:p75_{NTR} interaction induces acute synaptic elimination via p75^{NTR} expressed in the post-synaptic region.

A sequence for Homo sapiens p75 neurotrophin receptor having NCBI accession no. NP_002498.1 is shown below as SEQ ID NO:8.

| | |
|---|---|
| 1 | MGAGATGRAM DGPRLLLLLL LGVSLGGAKE ACPTGLYTHS |
| 41 | GECCKACNLG EGVAQPCGAN QTVCEPCLDS VTFSDVVSAT |
| 81 | EPCKPCTECV GLQSMSAPCV EADDAVCRCA YGYYQDETTG |
| 121 | RCEACRVCEA GSGLVFSCQD KQNTVCEECP DGTYSDEANH |
| 161 | VDPCLPCTVC EDTERQLREC TRWADAECEE IPGRWITRST |
| 201 | PPEGSDSTAP STQEPEAPPE QDLIASTVAG VVTTVMGSSQ |
| 241 | PVVTRGTTDN LIPVYCSILA AVVVGLVAYI AFKRWNSCKQ |
| 281 | NKQGANSRPV NQTPPPEGEK LHSDSGISVD SQSLHDQQPH |
| 321 | TQTASGQALK GDGGLYSSLP PAKREEVEKL LNGSAGDTWR |
| 361 | HLAGELGYQP EHIDSFTHEA CPVRALLASW ATQDSATLDA |
| 401 | LLAALRRIQR ADLVESLCSE STATSPV |

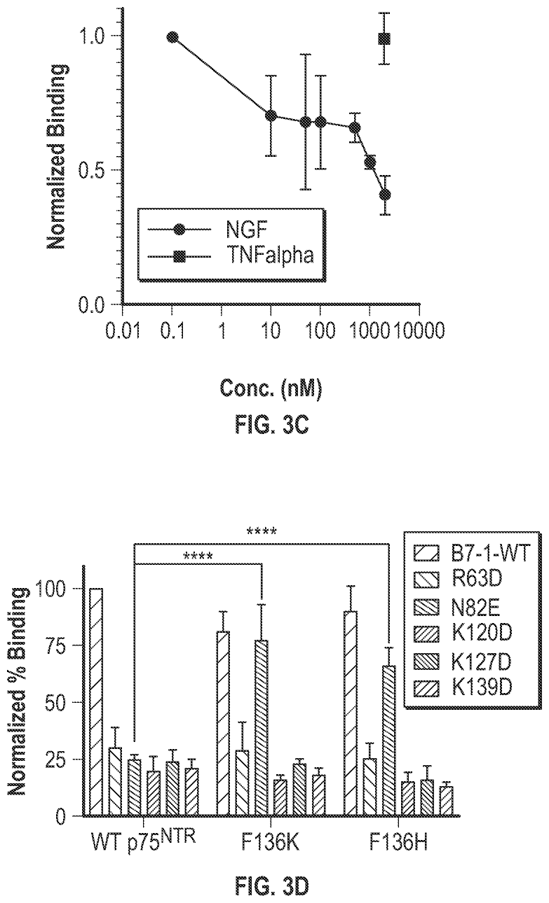

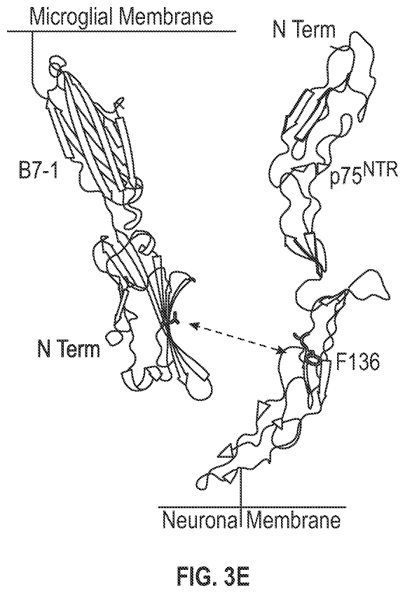

Eight point mutations at six positions in the p75^{NTR} protein with SEQ ID NO:8 exhibited significant reduction in binding of the p75_{NTR} protein to B7-1. The six positions of p75_{NTR} correspond to amino acids F136, S137, E147, P150, L165, and R182. For example, the F136D, S137A, S137D, E147D, P150A, P150D, L165A, and R182A mutant p75^{NTR} proteins exhibited greater than 50% reduction in binding to B7-1 (FIGS. 3A and 3D).

Of these mutations, only one (R182A) affected binding of the p75^{NTR} protein to PTPRF. However, mutations at positions L36, Y37, T38, and L49 of the p75NTR proteins also reduce or eliminate binding of the p75^{NTR} protein to PTPRF, while still maintaining binding to B7-1. These positions are highlighted in bold and with underlining in the p75^{NTR} SEQ ID NO:8 sequence below.

| | |
|---|---|
| 1 | MGAGATGRAM DGPRLLLLLL LGVSLGGAKE ACPTGLYTHS |
| 41 | GECCKACNLG EGVAQPCGAN QTVCEPCLDS VTFSDVVSAT |
| 81 | EPCKPCTECV GLQSMSAPCV EADDAVCRCA YGYYQDETTG |
| 121 | RCEACRVCEA GSGLVFSCQD KQNTVCEECP DGTYSDEANH |
| 161 | VDPCLPCTVC EDTERQLREC TRWADAECEE IPGRWITRST |
| 201 | PPEGSDSTAP STQEPEAPPE QDLIASTVAG VVTTVMGSSQ |
| 241 | PVVTRGTTDN LIPVYCSILA AVVVGLVAYI AFKRWNSCKQ |
| 281 | NKQGANSRPV NQTPPPEGEK LHSDSGISVD SQSLHDQQPH |
| 321 | TQTASGQALK GDGGLYSSLP PAKREEVEKL LNGSAGDTWR |
| 361 | HLAGELGYQP EHIDSFTHEA CPVRALLASW ATQDSATLDA |
| 401 | LLAALRRIQR ADLVESLCSE STATSPV |

In particular, L36D, Y37A, Y37D, T38D, and L49D mutations could all reduce p75^{NTR} binding to PTPRF without reducing B7-1 binding to p75^{NTR}. However, if a mutation at the R182 site is present in p75^{NTR}, then B7-1 binding as well as PTPRF binding, to this mutant p75_{NTR} protein is significantly reduced.

Mutations at positions M95, D162, E171, or D104 of the p75_{NTR} protein can reduce or eliminate hydrogen or electrostatic bonds with NGF, proNGF, or NT3, while binding to B7-1 is maintained. For example, the p75^{NTR} mutations that reduce or eliminate hydrogen or electrostatic bonds with NGF, proNGF, or NT3 can be the L36D, M95A, D104R, D162A, D162R, and/or E171A mutations. These positions are highlighted in bold and with underlining in the p75^{NTR} SEQ ID NO:8 sequence below.

| | |
|---|---|
| 1 | MGAGATGRAM DGPRLLLLLL LGVSLGGAKE ACPTGLYTHS |
| 41 | GECCKACNLG EGVAQPCGAN QTVCEPCLDS VTFSDVVSAT |
| 81 | EPCKPCTECV GLQSMSAPCV EADDAVCRCA YGYYQDETTG |
| 121 | RCEACRVCEA GSGLVFSCQD KQNTVCEECP DGTYSDEANH |
| 161 | VDPCLPCTVC EDTERQLREC TRWADAECEE IPGRWITRST |
| 201 | PPEGSDSTAP STQEPEAPPE QDLIASTVAG VVTTVMGSSQ |
| 241 | PVVTRGTTDN LIPVYCSILA AVVVGLVAYI AFKRWNSCKQ |
| 281 | NKQGANSRPV NQTPPPEGEK LHSDSGISVD SQSLHDQQPH |
| 321 | TQTASGQALK GDGGLYSSLP PAKREEVEKL LNGSAGDTWR |
| 361 | HLAGELGYQP EHIDSFTHEA CPVRALLASW ATQDSATLDA |
| 401 | LLAALRRIQR ADLVESLCSE STATSPV |

The forgoing p75NTR mutations can be combined or used alone to create soluble p75NTR affinity reagents that selectively bind B7-1, without affecting binding of neurotrophins or PTPRF. The sites described herein are useful for generating modified p75NTR proteins that can or cannot target B7-1. Such a modified p75NTR protein can be used as a therapeutic with reduced interactions only with B7-1 that does not interfere with neurotrophin based processes in the brain. Multimerizing this reagent could then generate a more effective therapeutic.

The p75^{NTR} protein with SEQ ID NO:8 has a transmembrane region at about amino acid positions 251-272 (LIPVYCSILA AVVVGLVAYI AF; SEQ ID NO:9). This p75^{NTR} transmembrane domain can be modified to generate a soluble form of p75^{NTR}. Use of the soluble form of p75^{NTR} as a therapeutic agent that binds B7-1 can mask cellular-bound B7-1 so that it does not interact with the cellular-bound form of p75NTR. Such a soluble form of p75_{NTR} interactions can therefore inhibit the biological effects of cell-bound B7-1:p75^{NTR} interactions. Modification of the p75^{NTR} transmembrane domain can include replacement or deletion of amino acids in the p75^{NTR} transmembrane domain. In some cases, modification of the p75^{NTR} transmembrane domain can include insertion of amino acids that have chemical and physical properties that are different from the amino acids in the wild type p75^{NTR} transmembrane domain (non-conservative amino acid substitutions).

In some cases, modified forms of the p75^{NTR} protein can include a deleted or modified transmembrane domain that does not insert into or associate with cellular membranes, especially neuronal cell membranes. Such soluble forms of p75^{NTR} can have mutations that reduce or eliminate binding to neurotrophins or PTPRF so that B7-1 is essentially the only factor that the soluble, mutant p75^{NTR} binds. The modified forms of p75^{NTR}, including the soluble forms of p75^{NTR} can also be linked to an antibody or an antibody fragment. In some cases, the modified forms of p75^{NTR}, including the soluble forms of p75_{NTR}, can be covalently linked to an Fc antibody fragment. Also, the antibody or antibody fragment can be linked to any region of the modified forms of p75_{NTR}, or to any region of the soluble forms of p75^{NTR}, in some cases the antibody or antibody fragment is linked to the C-terminal region of the modified or soluble forms of p75^{NTR}.

For example, in some cases the antibody or antibody fragment is linked to about 3-20 amino acids of the C-terminal region of the modified or soluble forms of p75^{NTR}.

Nucleic acid segments encoding wild type or modified forms of p75_{NTR} can be operably linked to a promoter to thereby generate an expression cassette useful for expressing the wild type or modified forms of p75_{NTR}. For example, cDNA sequence for the Homo sapiens the p75 neurotrophin receptor protein with SEQ ID NO:8 is shown below as SEQ ID NO:10 (and accorded NCBI accession no. NM_002507.4).

| | |
|---|---|
| 1 | AGAGCGAGCC GAGCCGCGGC CAGCTCCGGC GGGCAGGGGG |
| 41 | GGCGCTGGAG CGCAGCGCAG CGCAGCCCCA TCAGTCCGCA |
| 81 | AAGCGGACCG AGCTGGAAGT CGAGCGCTGC CGCGGGAGGC |
| 121 | GGGCGATGGG GGCAGGTGCC ACCGGCCGCG CCATGGACGG |
| 161 | GCCGCGCCTG CTGCTGTTGC TGCTTCTGGG GGTGTCCCTT |
| 201 | GGAGGTGCCA AGGAGGCATG CCCCACAGGC CTGTACACAC |
| 241 | ACAGCGGTGA GTGCTGCAAA GCCTGCAACC TGGGCGAGGG |
| 281 | TGTGGCCCAG CCTTGTGGAG CCAACCAGAC CGTGTGTGAG |
| 321 | CCCTGCCTGG ACAGCGTGAC GTTCTCCGAC GTGGTGAGCG |
| 361 | CGACCGAGCC GTGCAAGCCG TGCACCGAGT GCGTGGGGCT |
| 401 | CCAGAGCATG TCGGCGCCGT GCGTGGAGGC CGACGACGCC |
| 441 | GTGTGCCGCT GCGCCTACGG CTACTACCAG GATGAGACGA |
| 481 | CTGGGCGCTG CGAGGCGTGC CGCGTGTGCG AGGCGGGCTC |
| 521 | GGGCCTCGTG TTCTCCTGCC AGGACAAGCA GAACACCGTG |
| 561 | TGCGAGGAGT GCCCCGACGG CACGTATTCC GACGAGGCCA |
| 601 | ACCACGTGGA CCCGTGCCTG CCCTGCACCG TGTGCGAGGA |
| 641 | CACCGAGCGC CAGCTCCGCG AGTGCACACG CTGGGCCGAC |
| 681 | GCCGAGTGCG AGGAGATCCC TGGCCGTTGG ATTACACGGT |
| 721 | CCACACCCCC AGAGGGCTCG GACAGCACAG CCCCCAGCAC |
| 761 | CCAGGAGCCT GAGGCACCTC CAGAACAAGA CCTCATAGCC |
| 801 | AGCACGGTGG CAGGTGTGGT GACCACAGTG ATGGGCAGCT |
| 841 | CCCAGCCCGT GGTGACCCGA GGCACCACCG ACAACCTCAT |
| 881 | CCCTGTCTAT TGCTCCATCC TGGCTGCTGT GGTTGTGGGC |
| 921 | CTTGTGGCCT ACATAGCCTT CAAGAGGTGG AACAGCTGCA |
| 961 | AGCAGAACAA GCAAGGAGCC AACAGCCGGC CAGTGAACCA |
| 1001 | GACGCCCCCA CCAGAGGGAG AAAAACTCCA CAGCGACAGT |
| 1041 | GGCATCTCCG TGGACAGCCA GAGCCTGCAT GACCAGCAGC |
| 1081 | CCCACACGCA GACAGCCTCG GGCCAGGCCC TCAAGGGTGA |
| 1121 | CGGAGGCCTC TACAGCAGCC TGCCCCCAGC CAAGCGGGAG |
| 1161 | GAGGTGGAGA AGCTTCTCAA CGGCTCTGCG GGGGACACCT |
| 1201 | GGCGGCACCT GGCGGGCGAG CTGGGCTACC AGCCCGAGCA |
| 1241 | CATAGACTCC TTTACCCATG AGGCCTGCCC CGTTCGCGCC |
| 1281 | CTGCTTGCAA GCTGGGCCAC CCAGGACAGC GCCACACTGG |
| 1321 | ACGCCCTCCT GGCCGCCCTG CGCCGCATCC AGCGAGCCGA |
| 1361 | CCTCGTGGAG AGTCTGTGCA GTGAGTCCAC TGCCACATCC |
| 1401 | CCGGTGTGAG CCCAACCGGG GAGCCCCCGC CCCGCCCCAC |
| 1441 | ATTCCGACAA CCGATGCTCC AGCCAACCCC TGTGGAGCCC |
| 1481 | GCACCCCCAC CCTTTGGGGG GGGCCCGCCT GGCAGAACTG |
| 1521 | AGCTCCTCTG GGCAGGACCT CAGAGTCCAG GCCCCAAAAC |
| 1561 | CACAGCCCTG TCAGTGCAGC CCGTGTGGCC CCTTCACTTC |
| 1601 | TGACCACACT TCCTGTCCAG AGAGAGAAGT GCCCCTGCTG |
| 1641 | CCTCCCCAAC CCTGCCCCTG CCCCGTCACC ATCTCAGGCC |
| 1681 | ACCTGCCCCC TTCTCCCACA CTGCTAGGTG GGCCAGCCCC |
| 1721 | TCCCACCACA GCAGGTGTCA TATATGGGGG GCCAACACCA |
| 1761 | GGGATGGTAC TAGGGGGAAG TGACAAGGCC CCAGAGACTC |
| 1801 | AGAGGGAGGA ATCGAGGAAC CAGAGCCATG GACTCTACAC |
| 1841 | TGTGAACTTG GGGAACAAGG GTGGCATCCC AGTGGCCTCA |
| 1881 | ACCCTCCCTC AGCCCCTCTT GCCCCCCACC CCAGCCTAAG |
| 1921 | ATGAAGAGGA TCGGAGGCTT GTCAGAGCTG GGAGGGGTTT |
| 1961 | TCGAAGCTCA GCCCACCCCC CTCATTTTGG ATATAGGTCA |
| 2001 | GTGAGGCCCA GGGAGAGGCC ATGATTCGCC CAAAGCCAGA |
| 2041 | CAGCAACGGG GAGGCCAAGT GCAGGCTGGC ACCGCCTTCT |
| 2081 | CTAAATGAGG GGCCTCAGGT TTGCCTGAGG GCGAGGGGAG |
| 2121 | GGTGGCAGGT GACCTTCTGG GAAATGGCTT GAAGCCAAGT |
| 2161 | CAGCTTTGCC TTCCACGCTG TCTCCAGACC CCCACCCCTT |
| 2201 | CCCCACTGCC TGCCCACCCG TGGAGATGGG ATGCTTGCCT |
| 2241 | AGGGCCTGGT CCATGATGGA GTCAGGTTTG GGGTTCGTGG |
| 2281 | AAAGGGTGCT GCTTCCCTCT GCCTGTCCCT CTCAGGCATG |
| 2321 | CCTGTGTGAC ATCAGTGGCA TGGCTCCAGT CTGCTGCCCT |
| 2361 | CCATCCCGAC ATGGACCCGG AGCTAACACT GGCCCCTAGA |
| 2401 | ATCAGCCTAG GGGTCAGGGA CCAAGGACCC CTCACCTTGC |
| 2441 | AACACACAGA CACACGCACA CACACACACA GGAGGAGAAA |
| 2481 | TCTCACTTTT CTCCATGAGT TTTTTCTCTT GGGCTGAGAC |
| 2521 | TGGATACTGC CCGGGGCAGC TGCCAGAGAA GCATCGGAGG |
| 2561 | GAATTGAGGT CTGCTCGGCC GTCTTCACTC GCCCCCGGGT |
| 2601 | TTGGCGGGCC AAGGACTGCC GACCGAGGCT GGAGCTGGCG |
| 2641 | TCTGTCTTCA AGGGCTTACA CGTGGAGGAA TGCTCCCCCA |
| 2681 | TCCTCCCCTT CCCTGCAAAC ATGGGGTTGG CTGGGCCCAG |
| 2721 | AAGGTTGTGA TGAAGAAAAG TGGGCCAGTG TGGGAATGCG |
| 2761 | GCAAGAAGGA ATTGACTTCG ACTGTGACCT GTGGGGATTT |
| 2801 | CTCCCAGCTC TAGACAACCC TGCAAAGGAC TGTTTTTTCC |
| 2841 | TGAGCTTGGC CAGAAGGGGG CCATGAGGCC TCAGTGGACT |
| 2881 | TTCCACCCCC TCCCTGGCCT GTTCTGTTTT GCCTGAAGTT |
| 2921 | GGAGTGAGTG TGGCTCCCCT CTATTTAGCA TGACAAGCCC |
| 2961 | CAGGCAGGCT GTGCGCTGAC AACCACCGCT CCCCAGCCCA |
| 3001 | GGGTTCCCCC AGCCCTGTGG AAGGGACTAG GAGCACTGTA |
| 3041 | GTAAATGGCA ATTCTTTGAC CTCAACCTGT GATGAGGGGA |
| 3081 | GGAAACTCAC CTGCIGGCCC CTCACCTGGG CACCTGGGGA |
| 3121 | GTGGGACAGA GTCTGGGTGT ATTTATTTTC CTCCCCAGCA |
| 3161 | GGTGGGGAGG GGGTTTGGGG GCTTGCAAGT ATGTTTTAGC |
| 3201 | ATGTGTTTGG TTCTGGGGCC CCTTTTTACT CCCCTTGAGC |
| 3241 | TGAGATGGAA CCCTTTTGGC CCCCGAGCTG GGGGCCATGA |
| 3281 | GCTCCAGACC CCCAGCAACC CTCCTATCAC CTCCCCTCCT |
| 3321 | TGCCTCCTGT GTAATCATTT CTTGGGCCCT CCTGAAACTT |
| 3361 | ACACACAAAA CGTTAAGTGA TGAACATTAA ATAGCAAAGA |
| 3401 | AAGAAAAA |

This or related p75^{NTR} nucleic acids can be modified to include any of the mutations or deletions described herein, and the modified forms of p75^{NTR} can then be expressed to generate useful amounts of p75^{NTR} proteins for research and for use as therapeutic agents.

The p75^{NTR} protein alone or with other coreceptors can mediate several cellular functions that include cell death, survival, migration, and axonal growth inhibition. However, p75^{NTR} can be upregulated in invading cancer cells (e.g., glioma cells). Treatment of such cancer cells with either the mature neurotrophins (NGF, BDNF, or NT3) or a proneurotrophin (pro-NGF) that binds to and activates p75^{NTR} can enhance cancer cell migration. Hence, reduction in p75^{NTR} protein signaling can be beneficial for treatment and/or prevention of various diseases.

The p75_{NTR} protein with SEQ ID NO:8 also has a transmembrane region at about amino acid positions 251-272, with amino acid sequence (LIPVYCSILAAVVVGL VAYI AF; SEQ ID NO:9). Modification of this transmembrane region while retaining B7-1 binding can reduce or eliminates cell membrane insertion and intracellular signaling by p75^{NTR} protein. Such transmembrane modifications can include deletion, replacement, or insertion of one or more non-hydrophobic amino acids within the transmembrane region.

Isoforms and variants of B7-1 and p75_{NTR} proteins can be present amongst some individuals and populations. Such isoforms and variants of B7-1 and p75_{NTR} proteins and nucleic acids coding therefor can be used in the methods and compositions described herein so long as they are substantially identical to the B7-1 and p75_{NTR} proteins and nucleic acids sequences described herein. The terms “substantially identity” indicates that a polypeptide or nucleic acid has a sequence with between 55-100% sequence identity to a reference sequence, for example with at least 55% sequence identity, at least 60%, at least 70%, at least 80%, at least 90%, at least 95%, at least 96%, at least 97% sequence, at least 98%, at least 99% identity to a reference sequence over a specified comparison window. Optimal alignment may be ascertained or conducted using the homology alignment algorithm of Needleman and Wunsch, J. Mol. Biol. 48:443-53 (1970).

The isoforms or variants of B7-1 and p75_{NTR} proteins can be modified as described herein even though the isoforms or variants have some sequence variations relative to the sequences described herein.

The modified B7-1 and p75 neurotrophin receptor proteins described herein can have a variety of amino acids, and a variety of replacement mutations. For example, the modified B7-1 and p75 neurotrophin receptor proteins described herein can be modified to include any of the amino acids listed in Table 1.

TABLE 1

| | One-Letter | Common |
|---|---|---|
| Amino Acid | Symbol | Abbreviation |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |
| β-Alanine | | bAla |
| N-Methylglycine | | MeGly |
| (sarcosine) | | |
| Ornithine | | Orn |
| Norleucine | | Nle |
| Penicillamine | | Pen |
| Homoarginine | | hArg |
| N-methylvaline | | MeVal |
| Homocysteine | | hCys |
| Homoserine | | hSer |

The modified B7-1 and p75 neurotrophin receptor proteins described herein can, for example, have mutations that replace or delete one or more amino acids in the wild type protein. In some cases, one or more amino acids in the B7-1 and p75 neurotrophin receptor proteins can be replaced by a conservative amino acid. In other cases, one or more amino acids having physical and/or chemical properties that are different from the amino acid(s) that are present in the wild type B7-1 and p75 neurotrophin receptor proteins. Such non-conservative amino acid replacements can alter the binding properties and the activities of the B7-1 and p75 neurotrophin receptor proteins. For example, to change the binding and functions of the B7-1 and p75 neurotrophin receptor proteins, amino acids in specific positions within the B7-1 and p75 neurotrophin receptor proteins can be deleted or replaced by amino acids of another class, where the classes are identified in the following Table 2.

TABLE 2

| | |
|---|---|
| Classification | Genetically Encoded |

Hydrophobic

| | |
|---|---|
| Aromatic | F, Y, W |
| Apolar | M, G, P |
| Aliphatic | A, V, L, I |

Hydrophilic

| | |
|---|---|
| Acidic | D, E |
| Basic | H, K, R |
| Polar | Q, N, S, T, Y |
| Cysteine-Like | C |

For example, in some cases, a hydrophilic amino acid in a binding domain of B7-1 and p75 neurotrophin receptor proteins can be replaced with a hydrophobic amino acid. In some cases, hydrophobic amino acids or aliphatic amino acids can be replaced by hydrophilic amino acids, for example in the membrane domains or in the binding domains of B7-1 and p75 neurotrophin receptor proteins. In some cases, basic amino acids can be replaced with acidic amino acids; or polar amino acids can be replaced by aliphatic amino acids. For example, basic amino acids such a lysine or arginine, and aromatic amino acids such as tyrosine can, for example, be replaced by acidic amino acids or aliphatic amino acids. In some cases, polar amino acids such as asparagine can be replaced by acidic amino acids. Other types of replacements can also occur.

### Expression Systems

Nucleic acid segments encoding one or more wild type or mutant B7-1 protein, wild type or mutant p75_{NTR} protein, or an antibody that binds B7-1 or p75_{NTR} can be inserted into or employed with any suitable expression system. Commercially useful and/or therapeutically effective quantities of one or more wild type or mutant B7-1 proteins, wild type or mutant p75NTR proteins, or antibodies that bind B7-1 or p75_{NTR} can also be generated from such expression systems.

Recombinant expression of nucleic acids is usefully accomplished using a vector, such as a plasmid. The vector can include a promoter operably linked to nucleic acid segment encoding one or more of the modified or unmodified B7-1, p75_{NTR}, or antibody proteins.

The vector can also include other elements required for transcription and translation. As used herein, vector refers to any carrier containing exogenous DNA. Thus, vectors are agents that transport the exogenous nucleic acid into a cell without degradation and include a promoter yielding expression of the nucleic acid in the cells into which it is delivered. Vectors include but are not limited to plasmids, viral nucleic acids, viruses, phage nucleic acids, phages, cosmids, and artificial chromosomes.

A variety of prokaryotic and eukaryotic expression vectors suitable for carrying, encoding and/or expressing modified or unmodified B7-1, p75_{NTR}, or antibody proteins can be used. A variety of prokaryotic and eukaryotic expression vectors suitable for carrying, encoding and/or expressing modified or unmodified B7-1, p75_{NTR}, or antibody against such proteins can be employed. Such expression vectors include, for example, pET, pET3d, pCR2.1, pBAD, pUC, and yeast vectors. The vectors can be used, for example, in a variety of in vivo and in vitro situations.

The expression cassette, expression vector, and sequences in the cassette or vector can be heterologous. As used herein, the term “heterologous” when used in reference to an expression cassette, expression vector, regulatory sequence, promoter, or nucleic acid refers to an expression cassette, expression vector, regulatory sequence, or nucleic acid that has been manipulated in some way. For example, a heterologous promoter can be a promoter that is not naturally linked to a nucleic acid of interest, or that has been introduced into cells by cell transformation procedures. A heterologous nucleic acid or promoter also includes a nucleic acid or promoter that is native to an organism but that has been altered in some way (e.g., placed in a different chromosomal location, mutated, added in multiple copies, linked to a non-native promoter or enhancer sequence, etc.). Heterologous nucleic acids may comprise sequences that comprise cDNA forms. Heterologous coding regions can be distinguished from endogenous coding regions, for example, when the heterologous coding regions are joined to nucleotide sequences comprising regulatory elements such as promoters that are not found naturally associated with the coding region, or when the heterologous coding regions are associated with portions of a chromosome not found in nature (e.g., genes expressed in loci where the protein encoded by the coding region is not normally expressed). Similarly, heterologous promoters can be promoters that at linked to a coding region to which they are not linked in nature.

Viral vectors that can be employed include those relating to lentivirus, adenovirus, adeno-associated virus, herpes virus, vaccinia virus, polio virus, AIDS virus, neuronal trophic virus, Sindbis and other viruses. Also useful are any viral families which share the properties of these viruses which make them suitable for use as vectors. Retroviral vectors that can be employed include those described in by Verma, I. M., Retroviral vectors for gene transfer. In Microbiology-1985, American Society for Microbiology, pp. 229-232, Washington, (1985). For example, such retroviral vectors can include Murine Maloney Leukemia virus, MMLV, and other retroviruses that express desirable properties. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral nucleic acid.

A variety of regulatory elements can be included in the expression cassettes and/or expression vectors, including promoters, enhancers, translational initiation sequences, transcription termination sequences and other elements. A “promoter” is generally a sequence or sequences of DNA that function when in a relatively fixed location in regard to the transcription start site. For example, the promoter can be upstream of the nucleic acid segment encoding one or more modified or unmodified B7-1, p75_{NTR}, or antibody proteins, or fragments thereof.

A “promoter” contains core elements required for basic interaction of RNA polymerase and transcription factors and can contain upstream elements and response elements. “Enhancer” generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 by in length, and they function in cis. Enhancers function to increase transcription from nearby promoters. Enhancers, like promoters, also often contain response elements that mediate the regulation of transcription. Enhancers often determine the regulation of expression.

Expression vectors used in eukaryotic host cells (yeast, fungi, insect, plant, animal, human or nucleated cells) can also contain sequences for the termination of transcription, which can affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs.

The expression of anti-B7-1 or anti-p75_{NTR} antibodies from an expression cassette or expression vector can be controlled by any promoter capable of expression in prokaryotic cells or eukaryotic cells. Examples of prokaryotic promoters that can be used include, but are not limited to, SP6, T7, T5, tac, bla, trp, gal, lac, or maltose promoters. Examples of eukaryotic promoters that can be used include, but are not limited to, constitutive promoters, e.g., viral promoters such as CMV, SV40 and RSV promoters, as well as regulatable promoters, e.g., an inducible or repressible promoter such as the tet promoter, the hsp70 promoter and a synthetic promoter regulated by CRE. Vectors for bacterial expression include pGEX-5X-3, and for eukaryotic expression include pCIneo-CMV.

The expression cassette or vector can include nucleic acid sequence encoding a marker product. This marker product can be used to determine if a vector or expression cassette encoding the modified or unmodified B7-1, p75_{NTR}, or antibody therefor has been delivered to the cell and, once delivered, is being expressed. Marker genes can include the E. coli lacZ gene which encodes β-galactosidase, and green fluorescent protein. In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)).

Gene transfer can be obtained using direct transfer of genetic material, in but not limited to, plasmids, viral vectors, viral nucleic acids, phage nucleic acids, phages, cosmids, and artificial chromosomes, or via transfer of genetic material in cells or carriers such as cationic liposomes. Such methods are available in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

For example, the nucleic acid molecules, expression cassette and/or vectors encoding modified or unmodified B7-1, p75_{NTR}, or antibody therefor can be introduced to a cell by any method including, but not limited to, calcium-mediated transformation, electroporation, microinjection, lipofection, particle bombardment and the like. The cells can also be expanded in culture and then administered to a subject, e.g. a mammal such as a human. The amount or number of cells administered can vary but amounts in the range of about 10⁶ to about 10⁹ cells can be used. The cells are generally delivered in a physiological solution such as saline or buffered saline. The cells can also be delivered in a vehicle such as a population of liposomes, exosomes or microvesicles.

In some cases, the transgenic cell can produce exosomes or microvesicles that contain nucleic acid molecules, expression cassettes and/or vectors encoding modified or unmodified B7-1, p75_{NTR}, or antibody proteins, or a combination thereof. In some cases, the transgenic cell can produce exosomes or microvesicles that contain nucleic acid molecules that can include anti-B7-1 or anti-p75_{NTR} antibodies or fragments thereof to particular tissues. Microvesicles can mediate the secretion of a wide variety of proteins, lipids, mRNAs, and micro RNAs, interact with neighboring cells, and can thereby transmit signals, proteins, lipids, and nucleic acids from cell to cell (see, e.g., Shen et al., J Biol Chem. 286(16): 14383-14395 (2011); Hu et al., Frontiers in Genetics 3 (April 2012); Pegtel et al., Proc. Nat'l Acad Sci 107(14): 6328-6333 (2010); WO/2013/084000; each of which is incorporated herein by reference in its entirety. Cells producing such microvesicles can be used to express the modified or unmodified B7-1, p75_{NTR}, or antibodies therefor.

Transgenic vectors or cells with a heterologous expression cassette or expression vector can express the encoded modified or unmodified B7-1, p75_{NTR}, or antibodies directed against B7-1 or p75_{NTR}. Any of these vectors or cells can be administered to a subject. Exosomes produced by transgenic cells can also be used to administer modified or unmodified B7-1, p75_{NTR}, or antibodies therefore, or nucleic acids encoding any of the same to the subject.

Methods and compositions are therefore described herein that include modified or unmodified B7-1, p75_{NTR}, or antibody proteins.

### Screening Methods

Also described herein are screening methods that can be used to identify useful small molecules, polypeptides, antibodies, peptides, aptamers, darpins, affinity reagents, and other molecules that can inhibit B7-1:p75_{NTR} binding/interactions. Such useful small molecules, polypeptides, antibodies, peptides, aptamers, darpins, affinity reagents, and the like can be screened for binding p75_{NTR}, binding B7-1 protein, for inhibiting the binding of B7-1 to p75_{NTR}, for inhibiting loss of neuronal synapses, loss of neuronal synaptic connections, or a combination thereof. The one or more of the test agents can, for example, be small molecules, antibodies, antibody fragments, antibody-derived constructs, Fc-fusion proteins, proteins, peptides, aptamers, peptide aptamers, nucleic acid aptamers, darpins, nanobodies, affinity reagents, liposomes displaying at least one test agent, cells expressing at least one test agent on the cells' surface, and the like can also be evaluated as therapeutics for treating the neuronal diseases and conditions. For example, one or more of the small molecules, antibodies, antibody fragments, antibody-derived constructs, Fc-fusion proteins, proteins, peptides, aptamers, peptide aptamers, nucleic acid aptamers, darpins, nanobodies, affinity reagents, liposomes displaying at least one test agent, cells expressing at least one test agent on the cells' surface, and the like can also be tested to ascertain if they can reduce adverse symptoms of neuronal diseases or conditions.

The methods can involve contacting a B7-1 protein, a p75_{NTR} protein, or a combination thereof with one or more test agents with B7-1 and p75 neurotrophin receptor and measuring whether one or more of the test agents reduces B7-1 binding to p75 neurotrophin receptor. The B7-1 protein and/or a p75_{NTR} protein can be a wild type or modified form of B7-1 protein or p75_{NTR} protein.

Such contacting can involve incubating the one or more test agents with the B7-1 protein, the p75_{NTR} protein, or a combination thereof under conditions and for a time sufficient for biological interactions such as protein binding.

One or more of the test agents can be one or more small molecules, antibodies, antibody fragments, antibody-derived constructs, Fc-fusion proteins, proteins, peptides, aptamers, peptide aptamers, nucleic acid aptamers, darpins, nanobodies, affinity reagents, liposomes displaying at least one test agent, or cells expressing at least one test agent on the cells' surface.

In some cases, the B7-1 and p75 neurotrophin receptor can be expressed separately on different cells. In some cases, one or more of the B7-1 or p75 neurotrophin receptors are separately linked to different beads or carriers. In some cases, one or the other of the B7-1 and p75 neurotrophin receptor are tested in soluble form.

Measuring whether one or more of the test agents reduces B7-1 binding to p75 neurotrophin receptor can involve observing or quantifying whether a marker linked to B7-1 becomes localized to the p75 neurotrophin receptor, or vice versa. In some cases, B7-1 is linked to a detectable marker and the p75 neurotrophin receptor is not linked to a detectable marker. In some cases, the p75 neurotrophin receptor is linked to a detectable marker and the B7-1 is not linked to a detectable marker. In other cases, the p75 neurotrophin receptor and the B7-1 are linked to separate, distinguishable detectable markers.

Measuring whether one or more of the test agents reduces B7-1 binding to p75 neurotrophin receptor can involve observing or quantifying whether a B7-1-expressing cell binds to a p75 neurotrophin receptor-expressing cell, or vice versa. To facilitate detection and quantification of soluble or cell-bound B7-1 with soluble or cell-bound p75 neurotrophin receptor, the B7-1, p75 neurotrophin receptor, or the cells expressing the B7-1 or p75 neurotrophin receptor can have a detectable, label. For example, a cell expressing B7-1 can express a detectable marker or such a cell can become detectable by treating the B7-1-expressing cell with a reagent that specifically binds the B7-1-expressing cell. Similarly, a cell expressing p75 neurotrophin receptor can express a detectable marker or become detectable by treating the p75 neurotrophin receptor-expressing cell with a reagent that specifically binds the p75 neurotrophin receptor-expressing cell.

In some cases, the B7-1 and/or the p75 neurotrophin receptor can be bound to a bead, particle, or carrier instead of being in soluble form or instead of being expressed by/bound to a cell. Different detectable markers can be present on the beads, particles, or carriers to permit the bound B7-1 proteins and/or p75 neurotrophin receptors to be distinguished.

Binding between soluble and/or bound forms of B7-1 proteins and/or p75 neurotrophin receptors can be detected by flow cytometry, polyacrylamide gel electrophoretic (PAGE) separation under non-denaturing conditions, PAGE-SDS after crosslinking the results of binding assays, pulldown assays, and the like. Pulldown assays can involve use of one or more antibodies that can bind to and immobilize one of B7-1 or p75 neurotrophin receptor. The presence of the non-antibody bound B7-1 or p75 neurotrophin receptor on its binding partner (either p75 neurotrophin receptor or B7-1) can be detected using a labeled secondary antibody.

One or more of the test agents can be selected for further characterization if those test agents sufficiently inhibit binding between B7-1 and p75_{NTR}. For example, one or more of the test agents can be selected for further characterization if those test agents reduce B7-1 binding to p75 neurotrophin receptor by at least 25%, or at least 50%, or at least 60%, or at least 75%, or at least 80%, or at least 85%, or at least 90%, or at least 95%, or at least 96%, or at least 97%, or at least 98%, or at least 99% compared to a control assay mixture of the B7-1 and the p75 neurotrophin receptor without the one or more test agents. A test agent exhibiting such levels of inhibitory activity that is selected for further characterization can be referred to as a B7-1 blocking agent. Such a B7-1 blocking agent can bind to B7-1, or p75 neurotrophin receptor, or otherwise reduce or inhibit interaction between B7-1 and p75 neurotrophin receptor.

Any such B7-1 blocking agents can be characterized in a variety of ways. For example, one or more of the B7-1 blocking agent can be incubated with B7-1 in the presence of CD28, CTLA-4, PD-L1, or a combination thereof, and B7-1 binding to CD28, CTLA-4, PD-L1, or a combination thereof can be measured. When seeking B7-1 blocking agents that specifically inhibit B7-1:p75 neurotrophin receptor interactions, it may be more desirable to select B7-1 blocking agents that do not reduce B7-1 binding to CD28, CTLA-4, PD-L1, or a combination thereof. B7-1 blocking agents that do not significantly reduce B7-1 binding to CD28, CTLA-4, PD-L1, or a combination thereof can be referred to as B7-1-specific blocking agents.

B7-1 blocking agents and/or B7-1-specific blocking agents can be further characterized for their effects on neurons and/or neuronal synapses. As described herein, B7-1 binding to p75 neurotrophin receptor can lead to synaptic elimination when p75NTR is expressed in neuronal post-synaptic regions. Hence, for example, at least one B7-1 blocking agent and/or at least one B7-1-specific blocking agent can be incubated in a culture comprising B7-1 (e.g., as soluble B7-1 or as cell-bound B7-1) and neurons that express p75 neurotrophin receptor. The density of synaptic puncta of the neurons that express p75 neurotrophin receptor can then be measured. Synaptic density can be quantified by Golgi analysis or by detecting the presence of the post synaptic marker PSD95, and/or the presence of the dendritic marker MAP2. PSD-95 (postsynaptic density protein 95) also known as SAP-90 (synapse-associated protein 90) is a protein that in humans is encoded by the DLG4 (discs large homolog 4) gene. PSD-95 has a role in synaptic plasticity and the stabilization of synaptic changes during long-term potentiation. Such markers can be evaluated using immunofluorescence microscopy.

One or more of the B7-1 blocking agents or one or more of the B7-1-specific blocking agents that do not adversely affect synaptic puncta density can be selected as a useful B7-1 inhibitor. Such B7-1 inhibitors can maintain higher levels of synaptic puncta density when present in a culture that includes B7-1 (either soluble or cell-bound) and the neurons that express p75 neurotrophin receptor. Control cultures can be prepared for comparison where the control cultures include soluble B7-1 or B7-1-expressing cells as well as neurons that express p75 neurotrophin receptor without the B7-1-specific blocking agent.

The B7-1 inhibitors can be further characterized by administering one or more of the B7-1 inhibitor to an animal exhibiting symptoms of a neuronal condition or disease. In some cases, the animal is a model animal having or exhibiting symptoms of Alzheimer's disease, cognitive impairment, multiple sclerosis, stroke, neuronal injury, traumatic neural injury, spinal cord injury, lupus, Parkinson's disease, anxiety, schizophrenia, manic depression, delirium, dementia, mental retardation, Huntington's disease, or Tourette's syndrome.

The effects of the one or more B7-1 inhibitors on the neuronal condition or disease can be assessed by measuring whether the model animal has reduced symptoms of Alzheimer's disease, cognitive impairment, multiple sclerosis, stroke, neuronal injury, traumatic neural injury, spinal cord injury, lupus, Parkinson's disease, anxiety, schizophrenia, manic depression, delirium, dementia, mental retardation, Huntington's disease, or Tourette's syndrome, compared to a model animal that did not receive the at least one B7-1-specific blocking agent.

B7-1 inhibitors can therefore be selected as therapeutic agents for administration to humans and other primates. The B7-1 inhibitors can be administered for treatment of humans or primates having or exhibiting symptoms of Alzheimer's disease, cognitive impairment, multiple sclerosis, stroke, neuronal injury, traumatic neural injury, spinal cord injury, lupus, Parkinson's disease, anxiety, schizophrenia, manic depression, delirium, dementia, mental retardation, Huntington's disease, or Tourette's syndrome.

Examples of B7-1 inhibitors that can be therapeutic agents for administration to humans and other animals include abatacept, belatacept, modified CTLA-4 proteins that can block of B7 binding, modified CD28 proteins that can block of B7 binding, modified Inducible T Cell Costimulator Ligand (ICOSL) proteins that can block of B7 binding, or combinations thereof.

Such treatments can prevent, ameliorate, or correct pain (e.g., acute pain, chronic pain, neuropathic pain, nociceptive pain, radicular pain, thermal pain, or combinations thereof), neuronal inflammation, acute traumatic injuries to the nervous system, Alzheimer's disease, CNS disorders, cerebral ischemia, lupus, Parkinson's disease, multiple sclerosis, stroke, spinal cord injury, psychotic disorders, anxiety, schizophrenia, manic depression, delirium, dementia, several mental retardation, dyskinesias, Huntington's disease, Tourette's syndrome, or a combination thereof.

### Inhibitors

Also described herein are small molecules, peptides, aptamers, darpins, affinity reagents, and similar inhibitors of B7-1:p75_{NTR} binding or that reduce B7-1 or p75_{NTR} function. The small molecules, peptides, aptamers, darpins, affinity reagents, and similar inhibitors can be selected by performing the screening methods described herein. However, the peptide inhibitors can also include any peptides from B7-1 or p75_{NTR} that can reduce B7-1:p75_{NTR} binding or that reduce B7-1 or p75_{NTR} function. Such peptides can, for example, block or inhibit binding of B7-1 to PTPRF, CD28, CTLA-4, PD-L1, or a combination thereof. In another example, such peptides can block or inhibit binding of p75_{NTR} to NT3, NGF, proNGF or a combination thereof.

For example, small molecules based on NT3 or NGF peptides with amino acid sequences LSRKIGRT (SEQ ID NO:11) or LSRKAVRRA (SEQ ID NO:12) may be able make contacts with p75_{NTR} at the site that B7-1 contacts p75_{NTR}. Thus, a peptide that includes sequence LSRKIGRT (SEQ ID NO:11) or LSRKAVRRA (SEQ ID NO:12) can inhibit B7-1:p75 binding, as well as NT3/NGF/proNGF and likely other neurotrophin binding to p75_{NTR}.

Other examples of B7-1 inhibitors that can be therapeutic agents for administration to humans and other animals include abatacept, belatacept, modified CTLA-4 proteins that can block of B7 binding, modified CD28 proteins that can block of B7 binding, modified Inducible T Cell Costimulator Ligand (ICOSL) proteins that can block of B7 binding, or combinations thereof.

### Antibodies

Anti-B7-1 protein, or anti-p75_{NTR} antibodies can be generated as therapeutic agents that can inhibit B7-1 protein and p75_{NTR} protein interactions. Such antibodies can also be test agents for identifying B7-1 blocking agents and useful therapeutic agents.

Antibodies can be raised against various epitopes of the B7-1 protein, the p75_{NTR} protein, or a portion or epitope thereof. The antibodies contemplated as therapeutic agents for treatment pursuant to the methods and compositions described herein are preferably human or humanized antibodies and are highly specific for their B7-1 protein and p75_{NTR} protein targets.

As illustrated herein, mutation of B7-1 residues I36, T39, Y40, K43, 549, R63, Y65, N82, K120, Y121, K127, and K139 reduce B7-1 binding to p75_{NTR} (FIG. 2B; see SEQ ID NO:1). Of these, the N82, 192, and Y121 mutations specifically cause losses in B7-1 binding to p75NTR, and the N82, I92, and Y121 mutations do not affect interaction with CTLA-4 or CD28. In particular, mutation of the N82 residue substantially eliminates binds of B7-1 binding to p75NTR without adversely affecting B7-1 interaction with CTLA-4 or CD28.

While any antibody preparations that bind to B7-1 can be useful, antibodies that are specific for B7-1 but that do not adversely affect B7-1 interaction with CTLA-4 or CD28 may be more useful. Hence, in some cases, B7-1 epitopes in the three-dimensional vicinity of the N82, I92, or Y121 residues, or especially the N82 residue, of B7-1 may be selected for antibody preparation. For example, a peptide that includes a B7-1 sequence in the region of the N82, I92, or Y121 residue can be used as an antigen for generating anti-B7-1 antibodies. Other peptides having B7-1 sequences can also be used as antigens for generating anti-B7-1 antibodies.

Similarly, while any antibody preparations that bind to p75NTR can be useful, antibodies that are specific for p75NTR (and block binding to B7-1) but that do not adversely affect p75NTR interaction with PTPRF, NGF, proNGF, or NT3 can in some cases by more useful.

As described herein, several point mutations in the p75NTR protein with SEQ ID NO:8 exhibited greater than 50% reduction in binding of the p75_{NTR} protein to B7-1. These p75_{NTR} amino acid positions are F136, S137, E147, P150, L165, and R182 (FIG. 3A). However, mutation of the R182 amino acid affected binding of the p75_{NTR} protein to PTPRF. Hence, to obtain antibodies against p75_{NTR} that do not adversely affect p75NTR binding to PTPRF, NGF, proNGF, or NT3 peptide antigens can be used that include sequences near the F136, S137, E147, P150, or L165 positions of the p75_{NTR} protein. Such antigens can be useful for generating antibodies to p75_{NTR} that may inhibit binding of B7-1 to p75_{NTR} without adversely binding to PTPRF, NGF, proNGF, or NT3.

If antibodies are desired that block p75_{NTR} binding to PTPRF, peptide antigens with sequences in the region of R182, L36, Y37, T38, and L49 can be used to generate antibodies. Similarly, if antibodies are desired that block p75_{NTR} binding to NGF, proNGF, or NT3, peptide antigens with sequences in the region of M95, D162, E171, D104 of the p75_{NTR} proteins can be used. Such antibodies may still allow binding of p75_{NTR} to B7-1.

The antibodies may be monoclonal antibodies. Such antibodies may also be humanized or fully human monoclonal antibodies. The antibodies can exhibit one or more desirable functional properties, such as high affinity binding to p75_{NTR} or B7-1, or the ability to inhibit binding of B7-1 to p75_{NTR} protein.

Methods and compositions described herein can include antibodies that bind p75_{NTR} or B7-1 protein. The methods and compositions can use a combination of antibodies that bind to p75_{NTR} or B7-1, for example, combinations of antibodies can be used where each antibody type can separately bind p75_{NTR} or B7-1.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding portion” of an antibody (or simply “antibody portion”), as used herein, refers to one or more fragments of an antibody that retain the ability to specifically bind to an antigen (e.g. a peptide or domain of p75_{NTR} or B7-1). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody,” as used herein, is intended to refer to an antibody that is substantially free of other antibodies having different antigenic specificities (e.g., an isolated antibody that specifically binds p75_{NTR} or B7-1 is substantially free of antibodies that specifically bind antigens other than p75_{NTR} or B7-1 proteins. An isolated antibody that specifically binds p75_{NTR} or B7-1 may, however, have cross-reactivity to other antigens, such as isoforms or related p75_{NTR} or B7-1 proteins from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{L} and V_{H} regions of the recombinant antibodies are sequences that, while derived from and related to human germline V_{L} and V_{H} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds to human p75_{NTR} or B7-1” is intended to refer to an antibody that binds to human p75_{NTR} or B7-1 with a K_{D} of 1×10⁻⁷ M or less, more preferably 5×10⁻⁸ M or less, more preferably 1×10⁻⁸ M or less, more preferably 5×10⁻⁹ M or less, even more preferably between 1×10⁻⁸ M and 1×10⁻¹⁰ M or less.

The term “K_{assoc}” or “Kₐ,” as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D},” as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore™ system.

The antibodies of the invention are characterized by particular functional features or properties of the antibodies. For example, the antibodies bind specifically to human p75_{NTR} or B7-1. Preferably, an antibody of the invention binds to p75_{NTR} or B7-1 with high affinity, for example with a K_{D} of 1×10⁻⁷ M or less. The antibodies can exhibit one or more of the following characteristics:
(a) binds to human p75_{NTR} or B7-1 with a K_{D} of 1×10⁻⁷ M or less;
(b) inhibits the binding of p75_{NTR} to B7-1 or binding of B7-1 to p75_{NTR} protein;
(c) inhibits loss of synaptic density; or
(e) a combination thereof.

For example, the antibodies described herein can prevent greater than 30% binding, or greater than 40% binding, or greater than 50% binding, or greater than 60% binding, or greater than 70% binding, or greater than 80% binding, or greater than 90% binding of B7-1 to p75_{NTR}.

Assays to evaluate the binding ability of the antibodies to p75_{NTR} or B7-1 can be used, including for example, ELISAs, Western blots and RIAs. The binding kinetics (e.g., binding affinity) of the antibodies also can be assessed by standard assays known in the art, such as by Biacore™. analysis.

Given that each of the subject antibodies can bind to p75_{NTR} or B7-1, the V_{L} and V_{H} sequences can be “mixed and matched” to create other binding molecules that bind to p75_{NTR} or B7-1. The binding properties of such “mixed and matched” antibodies can be tested using the binding assays described above and assessed in assays described in the examples. When V_{L} and V_{H} chains are mixed and matched, a V_{H} sequence from a particular V_{H}/V_{L} pairing can be replaced with a structurally similar V_{H} sequence. Likewise, preferably a V_{L} sequence from a particular V_{H}/V_{L} pairing is replaced with a structurally similar V_{L} sequence.

Accordingly, in one aspect, the invention provides an isolated monoclonal antibody, or antigen binding portion thereof comprising:
(a) a heavy chain variable region comprising an amino acid sequence; and
(b) a light chain variable region comprising an amino acid sequence;
wherein the antibody specifically binds p75_{NTR} or B7-1.

### Compositions

The invention also relates to compositions containing active agents such as the B7-1 blocking agents, B7-1 inhibitors, p75_{NTR} or B7-1 binding agents, p75_{NTR} or B7-1 antibodies, modified p75_{NTR} or B7-1 polypeptides, and therapeutic agents described herein. Such active agents can be antibodies, nucleic acids encoding antibodies (e.g., within an expression cassette or expression vector), polypeptides, small molecules, peptides, aptamers, darpins, affinity reagents, and similar inhibitors, or a combination thereof. The compositions can be pharmaceutical compositions. In some embodiments, the compositions can include a pharmaceutically acceptable carrier. By “pharmaceutically acceptable” it is meant that a carrier, diluent, excipient, and/or salt is compatible with the other ingredients of the formulation, and not deleterious to the recipient thereof.

The composition can be formulated in any convenient form. In some embodiments, the compositions can include antibody, polypeptide, peptide, aptamer, or small molecule that can bind to p75_{NTR} or B7-1. In other embodiments, the compositions can include at least one nucleic acid or expression cassette encoding an antibody or polypeptide that can bind to p75_{NTR} or B7-1. In other embodiments, the compositions can include at least one nucleic acid or expression cassette encoding one or more modified p75_{NTR} or B7-1 polypeptides.

In some embodiments, the active agents of the invention (e.g., proteins, peptides, aptamers, darpins, affinity reagents, antibodies, nucleic acids encoding proteins, peptides, or antibodies (including for example, expression cassettes or expression vectors encoding such proteins, peptides, polypeptides, or antibodies), are administered in a “therapeutically effective amount.” Such a therapeutically effective amount is an amount sufficient to obtain the desired physiological effect, such reduction of at least one symptom of a neurological disease or condition. For example, active agents can reduce the short-term and the long-term symptoms of neurological disease or condition such as synaptic loss, inflammation, memory loss, tremors, psychological problems, or combinations thereof, by 5%, or 10%, or 15%, or 20%, or 25%, or 30%, or 35%, or 40%, or 45%, or 50%, or 55%, or 60%, or 65%, or %70, or 80%, or 90%, 095%, or 97%, or 99%, or any numerical percentage between 5% and 100%.

To achieve the desired effect(s), the active agents may be administered as single or divided dosages. For example, active agents can be administered in dosages of at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results. The amount administered will vary depending on various factors including, but not limited to, the type of active agents chosen for administration, the severity of the condition, the weight, the physical condition, the health, and the age of the mammal. Such factors can be readily determined by the clinician employing animal models or other test systems that are available in the art.

Administration of the active agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the active agents and compositions of the invention may be essentially continuous over a preselected period of time or may be in a series of spaced doses. Both local and systemic administration is contemplated.

To prepare the active agents, the active agents and other agents are synthesized or otherwise obtained, purified as necessary or desired. These agents can be suspended in a pharmaceutically acceptable carrier and/or lyophilized or otherwise stabilized. The agents, and combinations thereof can be adjusted to an appropriate concentration, and optionally combined with other desired agents. The absolute weight of a given agent included in a unit dose can vary widely. For example, about 0.01 to about 2 g, or about 0.1 to about 500 mg, of at least one agent, or a plurality of agents can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Daily doses of the agents of the invention can vary as well. Such daily doses can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day.

It will be appreciated that the amount of the agent for use in treatment will vary not only with the particular carrier selected but also with the route of administration, the severity of the condition being treated and the age and condition of the patient. Ultimately the attendant health care provider can determine proper dosage. In addition, a pharmaceutical composition can be formulated as a single unit dosage form.

Thus, one or more suitable unit dosage forms comprising the agent(s) can be administered by a variety of routes including parenteral (including subcutaneous, intravenous, intramuscular and intraperitoneal), oral, rectal, dermal, transdermal, intrathoracic, intrapulmonary and intranasal (respiratory) routes. The agent(s) may also be formulated for sustained release (for example, using microencapsulation, see WO 94/07529, and U.S. Pat. No. 4,962,091). The formulations may, where appropriate, be conveniently presented in discrete unit dosage forms and may be prepared by any of the methods well known to the pharmaceutical arts. Such methods may include the step of mixing the agents with liquid carriers, solid matrices, semi-solid carriers, finely divided solid carriers or combinations thereof, and then, if necessary, introducing or shaping the product into the desired delivery system. For example, the agent(s) can be linked to a convenient carrier such as a nanoparticle, albumin, polyalkylene glycol, or be supplied in prodrug form. The agent(s), and combinations thereof, can be combined with a carrier and/or encapsulated in a vesicle such as a liposome.

The compositions of the invention may be prepared in many forms that include aqueous solutions, suspensions, tablets, hard or soft gelatin capsules, and liposomes and other slow-release formulations, such as shaped polymeric gels. Administration of active agents can also involve parenteral or local administration of the in an aqueous solution or sustained release vehicle.

Thus, while the agents can sometimes be administered in an oral dosage form, that oral dosage form can be formulated so as to protect the antibodies, polypeptides, small molecules, nucleic acids, expression cassettes, and combinations thereof from degradation or breakdown before the antibodies, polypeptides, small molecules, nucleic acids encoding such polypeptides/antibodies, and combinations thereof provide therapeutic utility. For example, in some cases the agents can be formulated for release into the intestine after passing through the stomach. Such formulations are described, for example, in U.S. Pat. No. 6,306,434 and in the references contained therein.

Liquid pharmaceutical compositions may be in the form of, for example, aqueous or oily suspensions, solutions, emulsions, syrups or elixirs, dry powders for constitution with water or other suitable vehicle before use. Such liquid pharmaceutical compositions may contain conventional additives such as suspending agents, emulsifying agents, non-aqueous vehicles (which may include edible oils), or preservatives. The pharmaceutical compositions may take such forms as suspensions, solutions, or emulsions in oily or aqueous vehicles, and may contain formulatory agents such as suspending, stabilizing and/or dispersing agents. Suitable carriers include saline solution, encapsulating agents (e.g., liposomes), and other materials. The agents can be formulated in dry form (e.g., in freeze-dried form), in the presence or absence of a carrier. If a carrier is desired, the carrier can be included in the pharmaceutical formulation, or can be separately packaged in a separate container, for addition to the agents, after packaging in dry form, in suspension, or in soluble concentrated form in a convenient liquid.

Active agent(s) and/or other agents can be formulated for parenteral administration (e.g., by injection, for example, bolus injection or continuous infusion) and may be presented in unit dosage form in ampoules, prefilled syringes, small volume infusion containers or multi-dose containers with an added preservative.

The compositions can also contain other ingredients such as anti-inflammatory agents, antibacterial agents, antimicrobial agents, other monoclonal antibodies, and/or preservatives.

The present description is further illustrated by the following examples, which should not be construed as limiting in any way. The contents of all cited references (including literature references, issued patents, published patent applications as cited throughout this application) are hereby expressly incorporated by reference.

### Example 1: Materials and Methods

This Example describes some of the materials and methods used in the development of the invention.

### Tissue Culture and Transient Transfection

HEK 293 suspension cells were cultured in HEK Freestyle Media (Invitrogen, 12338018) grown at 37° C. in a humidified shaking platform incubator (Kuhner, Climo Shaker ISF4-X) with 5% CO₂. For transfection, cells were pelleted at 500×g and resuspended in fresh media. For small scale (1 mL cells at 1×106/mL) transient transfections performed in 24-well non-treated tissue culture plates, 2 μg Polyethylenimine (PEI, Fisher Scientific, AC178571000) was added to 0.5 μg diluted plasmid DNA in a final volume of 100 μL. For large-scale transfections (600 mL cells at 1×106/mL) carried out in 2 L baffled sterile shake flasks, 2 mg PEI was added to 400 μg diluted plasmid DNA.

B7-1 and p75NTR Site-Directed Mutagenesis

All site-directed mutagenesis of B7-1 and P75NTR was performed using high fidelity KOD Hot State polymerase, 2 mM dNTPs and 4 mM MgCl2 (EMD Millipore, 71086-3). The template used for the p75 mutagenesis included the coding sequence for full-length human p75 cloned between the XHOI and ECORI sites of the Clontech N1 GFP vector. For B7-1 the template used included the full-length native human B7-1 coding sequence cloned between the XHOI and ECORI sites of the Clontech N1 mCherry vector by In Fusion (Clontech).

For B7-1, positions selected for mutagenesis were based on the crystal structure of complex formed by human B7-1 and human CTLA-4 (PDB: 1I8L). Surface accessible residues in the IgG domain of B7-1 were identified using GetArea40 (56 positions total). For p75NTR, positions selected for mutagenesis were based on the crystal structure of complex formed by rat p75NTR and human NT3 (PDB: 3BUK). Mutagenesis was attempted such that each chosen position was mutated to an Ala and Asp/Glu/Arg residue. The overall mutagenesis success rate was ˜90%, and for some positions not all substitutions (A, D, E, R) were obtained. The sequence validated mutants were expression tested by transient transfection of 1 mL of suspension HEK 293 cells. Only those mutants exhibiting comparable expression to wild type B7-1 or p75NTR and correct plasma membrane localization were subsequently utilized in further binding studies, yielding a final library of 89 B7-1 mutants and 108 p75NTR mutants to assay.

### Cell-Cell Binding Experiments

Three mammalian expression display libraries were screened: a library of 479 combined Ig superfamily and TNFRSF constructs tagged with cytosolic GFP, a library of B7-1 point mutations tagged with cytosolic mCherry, and a library of p75NTR point mutations tagged with cytosolic mCherry. Query ligands were tagged with the opposite color cytosolic tag (mCherry for screening against the Ig and TNFR SFs library, and the p75NTR point mutant library, GFP for screening against the B7-1 point mutant library. For screening, libraries and query ligands were transfected in small scale as described above. Two days post-transfection, cells were diluted to 1*10⁶ cells/mL in PBS 0.2% BSA, pH 7.4. Binding reactions were setup in 96-well V-bottom plates by mixing equal volumes of challenger (scFv expressing cells) and library-expressing cells. After binding, cell-cell conjugates were analyzed by flow cytometry using a Hypercyte sample loader coupled to a BD Accuri flow cytometer or a SONY Spectral Analyzer (SA3800).

The percent bound was calculated as the number of double-positive events (GFP and mCherry) divided by the total number of cells. For mutagenesis studies, percent binding was calculated as the percent bound of a given mutant per percent bound of the wild type interaction.

### Purification of Recombinant Fc-Fusion Protein

To clone B7-1, CTLA-4, p75NTR, and PD-L1 Fc-fusion protein, full-length wild type or mutant ectodomains (B7-1: residues 35-233) were sub-cloned into a LIC vector containing a C601 terminal deca-his-tagged Fc domain (mIgG2a-His10 or hIgG1-His10). All expression constructs as well as mIgG2a and hIgG1 isotype control constructs were transiently expressed in 50 mL of ExpiHEK 293 suspension cells and transfected according to manufacturer guidelines. Seven days post transfection, the media was harvested, 50 mM MES was added to adjust to pH 6.5 and 100 mM Arg-Cl (pH 6.5) was added to enhance solubility. Fc-fusions were subsequently purified by Ni2+His60 chromatography (GE) using a batch binding method (3 mL resin bed volume) followed by gravity flow over a column. The Ni2+His60 resin was washed with 3 column volumes of wash buffer (50 mM MES pH 6.5, 100 mM Arg-Cl, 5 mM imidazole, 150 mM NaCl, 10% Glycerol) and the bound protein eluted with 5 mL the same buffer containing 500 mM imidazole. Nickel column elutes were concentrated and further purified by gel filtration on an S200 Sephadex column (MilliporeSigma, GE29321905) equilibrated with 50 mM MES pH 6.5, 100 mM Arg-Cl, 150 mM NaCl, 10% Glycerol. All recombinant proteins were used within one week of purification or were frozen at −80 C and only thawed one time. Frozen aliquots of protein were utilized but routinely checked for potential aggregation by analytical size chromatography). B7-1N82E-Fc eluted at the same volume as WT B7-Fc when purified using size exclusion chromatography, indicating the N82E mutation likely does not affect the overall stability of the B7-1 protein.

### Microbead Cell Flow Cytometry Binding Assay

For each experiment, 5 μL fluorescent protein A microbeads (either pink or yellow, Spherotech, PAFP-0552-5, PAFP-0558-5) were loaded with a mixture of 10 μg Fc fusion protein of interest (either B7-1 or p75NTR) of in a total volume of 500 μL 1×PBS 0.2% BSA, pH 7.4. The beads were incubated for 30 minutes at room temperature. Beads 622 were washed once by pelleting at 3000 g for 15 minutes and resuspended in 500 μL PBS 0.2% BSA, pH 7.4. Loaded beads were used within 12 hours. For bead:cell binding experiments, 5 μL of beads loaded with either B7-1-Fc, p75NTR-Fc, or Fc control, were incubated with 50,000 Freestyle HEK293 cells expressing ligands if interest in a total volume of 50 μL for 20 minutes. Bead:HEK cell binding was assessed by flow cytometry on a SONY Spectral Analyzer (SA 3800).

### Recombinant Protein:Bead Titration Experiments

For recombinant protein:bead titration experiments, hB7-1 (residues 35-233) was sub-cloned into a LIC vector containing a C-terminal 10×His and AVI tagged hIgG1 domain hIgG1-His10 and transiently expressed in ExpiHEK293 cells stably expressing a copy of the BirA gene, which enzymatically attaches a molecule of biotin to the AVI tag and was purified as described above. Biotinylation was confirmed by streptavidin pulldown and SDS-PAGE. 15 μg of B7-1-Fc-biotin was incubated with 5 μL of streptavidin coated beads (Bangs Lab, CP01003) in a total volume of 500 μL PBS 0.2% BSA, pH 7.4 at room temperature for 60 min. Beads were washed by pelleting at 1000 g for 5 minutes, and B7-1 loading was confirmed using an anti-B7-1 antibody (RND Systems, AF140) and flow cytometry. CTLA-4-mIgG2a, p75NTR-mIgG2a, and mIgG2a control were titrated onto 5 μL of B7-1 loaded beads in a 96 well plate at concentrations of 0-100 nM for 30 minutes, washed twice, and then incubated with an Alexa 647 labeled antibody (ThermoFisher, A-21235) against mIgG2a. Binding was detected by flow cytometry on a SONY Spectral Analyzer (SA 3800). Binding curves were fit using the equation Y=Bmax*Xh/(Kdh+Xh).

### Competition Experiments

For Competition experiments, fluorescent protein A beads were loaded (e.g., with B7-1 or p75) and incubated with cells expressing a ligand of interest (e.g., p75 or B7-1) as described above. CD28-Fc protein was purchased from Biolegend (755706). Competition was assessed by incubating increasing concentrations of ligand with the bead:cell binding reaction and determining binding of beads to cells by flow cytometry. Percent bound was calculated by divided the geomean fluorescence intensity of a given reaction by the geomean fluorescence of that reaction without competing protein.

### Flow Cytometry Protein:Cell Titration Assay

FACS titration assays were performed with B7-1-Fc and B7-1N82E Fc fusion proteins purified as described above. Cells were transfected with constructs expressing CD28, CTLA-4, or p75NTR, all with GFP tags. Three days post transfection cells were counted and diluted to 1×10⁶ cells/mL in 1×PBS 0.2% BSA, pH 7.4. Premixed solutions containing a final concentration of 1 μM Fc-fusion protein and 1.5 μM Alexa 647 goat anti-mouse secondary antibody (ThermoFisher, A-21235) were incubated on ice for 30 min. Subsequently, increasing amounts of the premixed solution was added to wells of a 96-well plate and the volume adjusted to 50 μL with 1×PBS and 100 μL of diluted cells (100,000 cells total) added to the wells. Binding was performed at room temperature for 1 hour, the cells washed 2× with 1×PBS 0.2% BSA by centrifugation and subsequently analyzed by Flow Cytometry on a SONY Spectral Analyzer (SA 3800). Gated live cells were sub-gated for GFP, and GFP-positive cells sub-gated for Alexa-647.

Data points represent the average of three independent experiments fit to the equation: Y=Bmax*Xh/(Kdh+Xh).

### Hippocampal Neuronal Cultures

For neuronal cultures, C57BL/6N mice from Charles 664 River Laboratories and p75NTR knockout mice (B6.129S4-Ngfrtm1Jae/J; Lee et al., Cell 69: 737-749 (1992)) from Jackson Laboratories were used. DIV18-21 primary hippocampal neurons were prepared from pregnant dams as described by Kaech, S. & Banker (Nat Protoc 1: 2406-2415 (2006)), with some modifications. Briefly, hippocampi from E18 mice were collected and digested with papain (Worthington Biochemical Corporation, Lakewood, NJ, USA; LS003127), in the presence of deoxyribonuclease I (Sigma; D4527), 1.5 mM CaCl2, and 0.75 mM EDTA solution in 37° C./5% CO₂ incubator for 25 min. The tissue was triturated and cells were plated on nitric acid treated, poly-L-lysine (Sigma; P2636) coated glass coverslips (Electron Microscopy Sciences, Hatfield, PA, USA; 72196-12). The cells were incubated for several hours in Minimal Essential Medium (GIBCO, Grand Island, NY, USA; 11095-080), supplemented with 1 mM sodium pyruvate (GIBCO; 11360070), 6 mM Glutamax (GIBCO; 35050061), 10% fetal bovine serum (GeminiBio; 100-500), 0.5% glucose, and 100 U ml-1 penicillin-streptomycin (GIBCO; 15140122). After allowing the cells to settle for at least 2 hours, the media was switched to Neurobasal medium (GIBCO; 21103049) supplemented with B27 (GIBCO, 17504-044), 1 mM sodium pyruvate, 6 mM Glutamax, 100 U ml-1 penicillin-streptomycin, and 4 μM cytosine-1-b-D-arabinofuranoside (Sigma; C6645) and maintained in culture for 18-21 days without any media changes/additions. Neurons were starved for 2-3 hours in Neurobasal medium supplemented with 0.5% glucose. Following starvation, the neurons were treated with 0.7 μM (40 ng ml-1) of B7-1 and B7-2 proteins (Sino Biological; 10698-H03H & 10699-H03H) at 37° C./5% CO2. 2 hours after protein application, the neurons were briefly washed with prewarmed Hank's Balanced Salt Solution (GIBCO; 14175095) and fixed with pre-warmed 4% paraformaldehyde/4% sucrose solution for 15 minutes at room temperature.

### Mixed Culture Assay

The mixed culture assays were prepared as described (Biederer, T. & Scheiffele, Nat Protoc 2: 670-676 (2007)) with modifications. HEK 293 cells were washed with 10 ml warm neurobasal medium supplemented with B27, 1 mM sodium pyruvate, 6 mM Glutamax, 100 U ml-1 penicillin-streptomycin and 4 μM cytosine-1-b-D-arabinofuranoside per 100×20 culture dish to prevent HEK 293 overgrowth. HEK 293 cells were triturated without trypsinization, counted and seeded at a density of 30×103 per 12 mm glass coverslip with hippocampal neuronal cultures. The mixed culture was incubated for 4 hours and fixed with prewarmed 4% paraformaldehyde, 4% sucrose solution for 15 min at room temperature.

### Immunocytochemistry

For immunocytochemistry of the hippocampal neuronal cultures and mixed culture assays, cells were permeabilized and blocked with 3% fetal bovine serum, 3% BSA, and 0.1% Triton X-100 in phosphate buffered saline for 30 minutes at room temperature. Primary antibodies were applied for 1 hour at room temperature, and subtype-specific Alexa fluorescent secondary antibodies were added for 20 minutes at room temperature. Coverslips were mounted with ProLong Gold antifade reagent (Invitrogen; P36934). The following primary antibodies were used: anti-PSD-95 (Abcam; ab2723), anti-MAP2 (Abcam; ab92434), and anti-p75 (R&D Systems; AF1157). Secondary antibodies were Alexa Fluor antibodies (Life Technologies, Norwalk, CT, USA), except for the 405 nm fluorescent DyLight (Jackson ImmunoResearch Laboratories, West Grove, PA, USA; 102649-302). For visualization of actin cytoskeleton, Alexa Fluor 546 and 647 phalloidin (Life Technologies, Thermo Fisher Scientific; A22283 & A30107) were used.

### Fluorescence Microscopy

Recombinant protein assay images were acquired on a 709 n inverted microscope, Nikon Eclipse TE2000-U, and light source was PhotoFluor from Chroma. The objective used was PlanApo 60xA/1.40 oil Nikon. Acquisition settings were kept consistent across all conditions within a given experiment. For high magnification, mixed culture assay images were taken on a Zeiss Cell Observer SD confocal with a Yokagawa CSU-X1 spinning disk, Plan-Apochromat 63×/1.4 M27 objective paired with a 1.2× adapter to a Photometrics Evolve 512 EMCCD camera was used for image acquisition. The laser lines used were 405 nm, 488 nm, 561 nm and 639 nm.

### Quantification and Statistical Analysis

For quantitative assessment of synaptic puncta, the PSD95 channel was analyzed and processed with FIJI software. Secondary and tertiary dendritic branches were selected blind, based on the actin and MAP2 channels. Background was subtracted and a threshold was applied. PSD95 puncta on secondary and tertiary dendritic branches were counted from particles between 0.03-10 pixels microns in size. Lastly, the length of the branch was measured. Experiments were performed at least in triplicate and independently replicated three times with similar results. All data was acquired and analyzed in a blinded fashion, and experiments were repeated by different individuals to ensure reproducibility. All data were analyzed with GraphPad Prism 5.0 software (San Diego, CA, USA). Experimental data were statistically analyzed by one-way ANOVA with Bonferroni post hoc tests to control for multiple comparisons (for three or more group comparisons), or two-way ANOVA with Bonferroni post hoc tests (to assess statistical significance between means), as indicated within individual figure legends. In figures, asterisks denote statistical significance marked by *p<0.05, **p<0.01, ***p<0.001, and “n.s.” indicates no statistical significance. All statistical parameters are presented as means±SEM (Standard Error of Mean).

### MAP2 Quantitation

For quantitative assessment of MAP2 integrity, the MAP2 channel alone was used and processed with imageJ software. Background was subtracted and a threshold was applied. Secondary and tertiary dendritic branches were traced with a linear path using the actin channel. The plot profile tool was then used to quantify the threshold MAP2 signal along the dendrite. The area under the curve of each linear path was then divided by 255*path length and multiplied by 100 in order to determine % continuity of MAP2 signal through the dendrite. Data was batch analyzed in Python.

### Example 2: B71 Directly Interacts with p75_{NTR}

Cells expressing human B7-1-mCherry were screened against a library of cells expressing 395 members of the human immunoglobin (Ig) and TNFR superfamily that were tagged with GFP (see FIG. 1A). The screen revealed trans-interactions between B7-1 and both CTLA-4 and CD28, which was expected. However, the screen also identified a previously uncharacterized interaction between B7-1 and p75NTR (is also known as TNFR16) (FIG. 1A-1B). Binding to PD-L1 expressing cells was not observed, as this interaction occurs in cis.

p75NTR expressing cells were then screened against the same library of cells expressing 395 members of the human immunoglobin (Ig) and TNFR superfamily. Again B7-1 binding to the p75NTR expressing cells was observed. In addition, the p75NTR expressing cells interacted with protein tyrosine phosphatase receptor type F (PTPRF; FIG. 1B). No binding was observed between human B7-1 and other members of the TNFR superfamily such as TNFR2, HVEM, FAS, and other TNFR proteins. No binding was also observed between p75NTR and other members of the human B7 family such as B7-2, ICOSL., PD-L.1, and other B7 family members.

To confirm that the B7-1:p75NTR mediated cell conjugation is the result of direct contacts between B7-1 and p75NTR, binding between B7-1 and p75 was validated by titrating recombinant p75-mIgG2A and CTLA-4-mIgG2A protein onto streptavidin beads coated with B7-1-hIgG1-biotin. B7-1:p75NTR binding was assessed using an in vitro flow cytometry using an anti-mIgG2A antibody. As illustrated in FIG. 1C, flow cytometry titrations demonstrate an approximate 30-fold higher EC50 between B7-1:p75NTR (16 nM) than B7-1:CTLA-4 (10.6 nM) using recombinant dimeric proteins.

While p75NTR sequences are highly conserved in mammals and many vertebrates (Mischel et al. J Biol Chem 276, 11294-11301 (2001)), BY-1 sequencing indicated that B7-1 has greater variability between species. Therefore, mammalian B7-1 sequences possessing varying degrees of sequence conservation were screened against p75NTR from human, mouse, and rat. As shown in FIG. 1D, while binding between B7-1 and human p75NTR is conserved in humans and old world monkeys, it was not detected in any of the other B7-1 homologues examined, including sperm whales, which express the closest non-primate B7-1 sequence identity to humans, and the commonly used laboratory model organisms mice and rats. Conversely, human/primate B7-1 did bind to either mouse or rat p75NTR was noted, likely due to high sequence conservation of p75NTR across mammalian species. However rodent B7-1 (rat or mouse) does not interact with rodent p75NTR, suggesting that the interaction is a recent primate synapomorphy.

These results indicated that if cross species studies are contemplated, evaluation of the biological effects of the B7-1:p75 interaction in vitro or in vivo requires the utilization of the human B7-1 (hB7-1) protein with murine neurons. Moreover, these results indicated that murine models of aging/inflammation/injury will fail to elucidate the potential effects of a human B7-1:p75 interaction, unless a knock-in mouse, expressing human B7-1 is utilized. Such a humanized mouse model is described below.

### Example 3: The Binding Site of p75NTR on B7-1 Overlaps with the Binding Site of CD28 and CTLA-4, but not PD-L1

To evaluate the molecular determinants of the human B7-1:p75NTR complex, an alanine/aspartic acid/arginine scanning method was used to generate structural information about transmembrane receptor:ligand complexes. The binding site for p75NTR on B7-1 was assayed against a library of cells HEK293F cells, each cell type individually expressing one of ninety-two (92) different B7-1 IgV domain mutants. Solvent accessible residues of BY-1 were selected for mutations where the selected residues were replaced with alanine, aspartic acid/glutamic acid, or arginine.

B7-1 mutant proteins expressed on HEK293F cells were assayed for their ability to bind CTLA-4-GFP, CD28-GFP, or p75NTR-GFP that were separately expressed on HEK293F cells. In total, eighteen mutations exhibited decreased binding to one or more binding partner, and eleven showed decreased binding to p75NTR (FIG. 2A-2B). The I36A, I36D, T39A, K40D, E41A, K43D, V45D, S49A, R63D, Y65A, E69A, K120D, K127D, L131D, and K139D mutants of B7-1 resulted in greater than 25% losses in B7-1 binding to CD28 and CTLA-4 (FIG. 2A). The majority of these mutations (all except for K139D, T39A, V45D, and S49A) were either directly consistent with positions in the CTLA-4 binding site observed in the crystal structure of the CTLA-4:B7-complex or were present on the GFCC′C″ face of B7-1 (FIG. 2A).

The B7-1 mutations I36D, T39A, Y40D, K43D, S49A, R63D, Y65A, N82E, K120D, Y121D, K127D, and K139D decreased B7-1 binding to p75NTR by more than 25% (FIG. 2B). Of these, N82E, I92D, and Y121D specifically caused losses in binding to p75NTR, but do not affect interaction with CTLA-4 or CD28. Mapping these twelve mutations to the crystal structure of the B7-1:CTLA-4 complex demonstrated clustering at and near the CTLA-4 binding site on the GFCC′C″ face of B7-1 (FIG. 2B-2C). N82 is located on middle of C″ strand and Y121 is located at the top of the F strand of BY-1, both of which are outside the putative CTLA-4/CD28 binding site. I92 is located on the dimerization face of B7-1, but directly behind N82 on the C″ strand. Mutating the I92 residue may cause perturbations to the C″ strand which alters p75NTR binding. These results indicate that the B7-1 recognition surfaces for CTLA-4, CD28, and p75NTR physically overlap, but the p75NTR recognition surface is more expansive than that of CD28 and CTLA-4 (FIG. 2C).

The B7-1 binding site for p75NTR was additionally compared to the B7-1 binding site for PD-L1. Recent reports indicate that cells expressing B7-1 do not bind cells expressing PD-L1, and that B7-1 binds to PD-L1 in cis on the same cell surface (Sugiura et al, Science 364: 558 (2019); Chaudhri et al. Cancer Immunol Res 6: 921-929 (2018); Garrett-Thomson et al. PloS one 15, e0233578 (2020)). The PD-L1 binding site was therefore mapped onto human B7-1 using recombinant human PD-L1-Fc, which can adopt an appropriate pose. An incubation mixture were prepared with 1 μM PD-L1-Fc and cells expressing the library of B7-1 point mutations. B7-1:PD-L1 binding was detected by flow cytometry.

As shown in Table 3 below, many of the selected B7-1 point mutations significantly reduced binding to PD-L1-Fc.

TABLE 3
B7-1 point mutations that significantly reduce binding to PD-L1-Fc

| B7-1 | % | | |
|---|---|---|---|
| Mutation | Binding | n= | St. Dev. |
| SER49ALA | 38.9% | 2 | 0.27 |
| ASN53ALA | 21.0% | 3 | 0.14 |
| ASN53ASP | 30.7% | 4 | 0.17 |
| SER55ALA | 34.4% | 3 | 0.29 |
| GLU57ARG | 27.2% | 3 | 0.24 |
| ASN89ALA | 17.8% | 3 | 0.19 |
| ASN89ASP | 20.1% | 3 | 0.24 |
| ILE92ALA | 19.7% | 4 | 0.13 |
| ILE92ASP | 27.3% | 3 | 0.21 |
| ALA105ASP | 35.6% | 4 | 0.16 |
| LYS139ALA | 33.0% | 3 | 0.32 |
| LYS139ASP | 92.3% | 3 | 0.17 |

Eleven B7-1 mutants (S49A, N53A, N53D, S55A, E57R, N89A, N89D, I92A, I92D, A104D, K139A) had greater than 70% loss in binding to PD-L1-Fc compared to wild-type B7-1. These residues are distinct from the p75NTR recognition site. These results are consistent with previous data generated by the inventors and others demonstrating that the PD-L1 and CTLA-4/CD28 binding sites are located on opposites face of the human B7-1 IgV domain. Of the eighteen B7-1 mutants with decreased binding to either CTLA-4, CD28 or p75NTR, sixteen bound PD-L1 with similar to wild-type B7-1 (see Table 3 above). Overall, these results indicate that the PD-L1 binding site on B7-1 and the p75NTR binding site on B7-1, are unique.

To confirm that the B7-1:p75NTR binding interface overlaps with the CD28 and CTLA-4 binding interfaces, in vitro ligand competition experiments were used. B7-1-Fc was conjugated to fluorescent protein A beads, and binding to HEK293F cells expressing p75NTR was detected using flow cytometry. Addition of increasing concentrations of CTLA-4-Fc or CD28-Fc abrogated B7-1:p75NTR binding, while isotype control protein did not (FIG. 2D-2E). These results indicate that binding interfaces for CD28, CTLA-4 and p75NTR are shared/overlapping, as suggested by mutagenesis mapping data. CTLA-4-Fc is a more effective inhibitor of B7-1:p75 binding than is CD28-Fc. Such inhibition is likely due to the approximate 10-fold higher affinity of CTLA-4 for B7-1 than the affinity of CD2839 for B7-1.

To further validate the B7-1 requirements for binding to p75NTR, a recombinant soluble form of a B7-1 point mutation (N82E) was generated that specifically exhibited essentially no binding to p75NTR, but that maintained near wild-type binding to CTLA-4, CD28 and PD-L1. Increasing amounts of B7-1-Fc and B7-1N82E-Fc were titrated into cells expressing CTLA-4, CD28, or p75NTR, and EC50s for each were calculated. B7-1-Fc bound to cells expressing either CD28, CTLA-4, or p75NTR (FIG. 2F). While B7-1N82E-Fc bound CTLA-4 and CD28 expressing cells with similar affinity to B7-1-Fc (EC50 about 9 or 10 nM), binding between B7-1N82E-Fc and p75NTR-expressing cells could not be detected. Therefore, the asparagine at B7-1 position 82 is critical for p75NTR binding.

### Example 4: The p75NTR Binding Site for B7-1 Partially Overlaps with the p75NTR Binding Site Neurotrophin

The p75NTR binding site for B7-1 was mapped using the same approach described for determining the B7-1 binding site for p75NTR. Solvent accessible residues on the surface of the p75NTR extracellular domain (calculated using GetArea40) were mutated to alanine, aspartic acid/glutamic acid, or arginine in a construct encoding full length p75NTR fused to the cytoplasmic C-terminus of GFP. Eighty (80) residues were identified, and 114 different p75NTR-GFP point mutants were successfully generated. This library was screened against B7-1-Fc to identify the p75NTR point mutations that impacted binding. In order to ensure the structural integrity of point mutations, the library was also screened against cells expressing PTPRF mCherry expressing cells.

In total, eight p75NTR point mutations covering seven residues exhibited greater than 50% losses in binding to B7-1 (F136D, S137A, S137D, E147D, P150A, P150ID, L165A, R182A) (FIG. 3A). Of these mutations, only one (R182A) affected binding to PTPRF, confirming the overall structural integrity of the other seven mutations. Mutations that affected p75NTR binding to B7-1 mapped to the more membrane proximal region of p75NTR (CRD3 and CRD4 domains) and one of these mutations (E147A) overlaps with the neurotrophin binding site as determined by x-ray crystallography (FIG. 3A-3B). These data suggest that on p75NTR the B7-1 and neurotrophin binding sites modestly overlap but have distinct recognition surfaces. This observation was confirmed with competition studies using soluble NGF at high concentrations (FIG. 3C).

To further confirm the results of the mutagenesis study, and to provide additional molecular detail about the B7-1:p75NTR binding interface, a modest screen for “salt bridge suppressors” that restored binding to defective mutants was conducted. p75NTR residues of apparent importance for B7-1 binding (F136, S137, P150, L165, P166, R182) were mutated in a series of constructs to lysine and histidine. These p75NTR point mutants were screened against aspartic and glutamic acid B7-1 mutants at positions R63, N82, K120, K127, and K139 that had significantly reduced binding to wild-type p75NTR. As shown in FIG. 3D, B7-1 N82E binding was rescued when using the p75NTR F136K and p75NTR F136H mutant proteins. None of the other p75NTR mutant. Similarly, none of the other B7-1 D/E mutants bound to any of the p75NTR K/H mutants. Table 4 below shows the percent binding between the p75NTR mutants listed in the first column to the left for the B7-1 (CD80) wild type or mutant proteins listed along the top row.

TABLE 4
Percent binding between the p75NTR mutants (first column)
for B7-1 (CD80) wild type or B7-1 mutant proteins (top row)

| | Wildtype | | | | | |
|---|---|---|---|---|---|---|
| P75NTR | CD80 | ARG63 | ASN82 | LYS120 | LYS127 | LYS139 |
| Mutation | Binding | ASP | GLU | ASP | ASP | ASP |
| F136K | 81% | 29% | 77% | 16% | 23% | 18% |
| F136H | 90% | 25% | 66% | 15% | 16% | 13% |
| P150K* | 69% | 14% | 13% | 17% | 15% | 11% |
| P150H* | 38% | 13% | 10% | 16% | 11% | 14% |
| LEU165K* | 83% | 18% | 11% | 20% | 19% | 19% |
| LEU165H* | 64% | 26% | 15% | 19% | 25% | 25% |
| ARG166H* | 66% | 8% | 25% | 12% | 26% | 26% |
| WT | 100% | 30% | 25% | 20% | 26% | 21% |

These results indicate that when B7-1 and p75NTR bind, the N82 residue of B7-1 is in close proximity to the F136 residue of p75NTR (FIG. 3E).

### Example 5: B7-1 Disassembles Synapses Through a p75NTR Dependent Mechanism

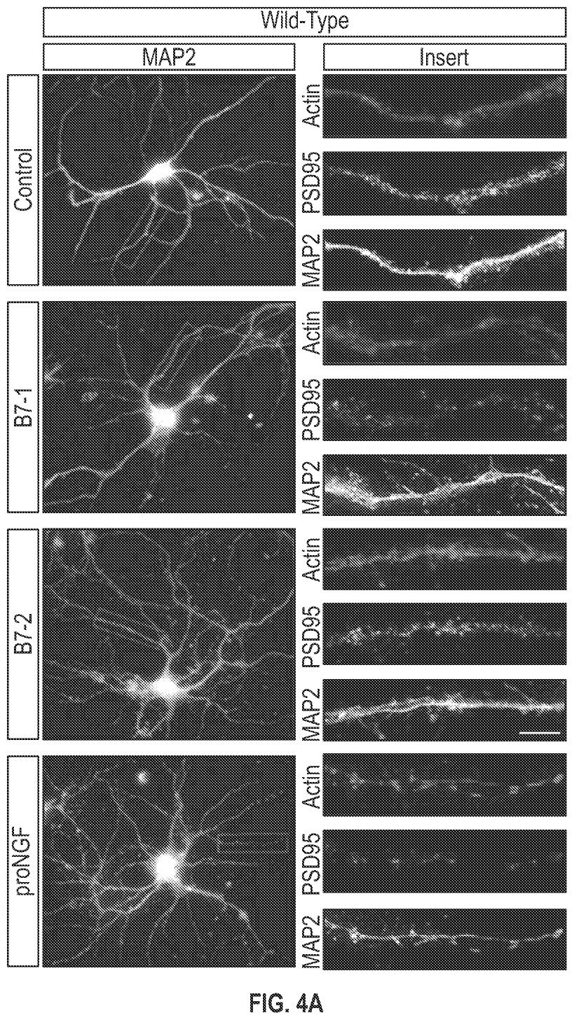

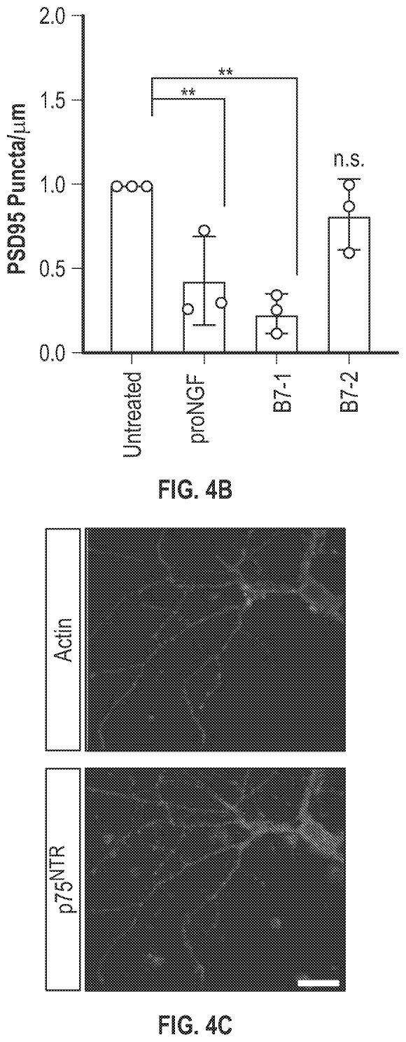

As B7-1:p75NTR interactions could occur in vivo between microglia and neurons, and because p75NTR biology has been best characterized in the nervous system, the effect of B7-1 on mature, p75NTR-expressing neurons was examined (FIG. 4). The inventors previous work demonstrated that pro forms of neurotrophins and a variant form of the BDNF pro-domain can induce acute dendritic spine collapse and PSD95 relocalization in primary hippocampal neurons in vitro by signaling through actin cytoskeletal regulators. This assay of dendritic spine collapse was chosen as the loss of synaptic spines is a hallmark of the inflammatory and neurodegenerative conditions under which B7-1 and p75NTR are co-expressed.

The inventors' prior studies localized p75NTR to post-synaptic sites both in vivo and in cultured hippocampal neurons (Giza et al. Neuron 99, 163-178.e166 (2018)). To determine the effects of B7-1 on p75NTR expressing neurons, the inventors took advantage of the cross-species reactivity between human B7-1 and mouse p75NTR. Recombinant B7-1-Fc (750 nM—to ensure saturation of the p75NTR receptor), B7-2-Fc (750 nM), or proNGF (10 nM) was added to cultured primary DIV18 hippocampal mouse neurons for two hours. Following fixation, actin localization, the presence of the post synaptic marker PSD95, and the presence of the dendritic marker MAP2, were evaluated using immunofluorescence microscopy.

As illustrated in FIG. 4A-4B, within two hours, neurons treated with B7-1-Fc or proNGF (as a positive control) exhibited a significantly lower density of PSD95-positive dendritic spines than control treated cells, and neurons treated with B7-2-Fc were unaffected. Expression of p75NTR was confirmed using immunofluorescence microscopy (FIG. 4C). These results indicate that changes in PSD95 density upon treatment with B7-1-Fc are not a result of interactions with CD28 or CTLA-4. Hence, B7-1 disassembles synapses through interactions with p75NTR.

To further establish that B7-1-mediated synapse disassembly is dependent on p75NTR binding, hippocampal neurons from p75NTR knockout (p75^{-/-}) mice were cultured and one of the following were added B7-1-Fc, B7-2-Fc, or proNGF. The density of PSD95-positive puncta was then analyzed after two hours. In p75^{-/-} neurons, addition of neither B7-1 nor proNGF exhibited altered the density of PSD95-positive puncta, indicating that B7-1-mediated dendritic spine collapse is dependent upon binding to and signaling through p75NTR.

Previous work has indicated that proneurotrophin-mediated or variant BDNF prodomain-mediated p75NTR signaling in neurons requires the interaction of the prodomain with co-receptors, Sortilin or SorCS2. However, no binding could be detected between cells expressing B7-1 and cells expressing Sortilin, SorCS1, SorCS2, or SorCS3. The differences in the binding interface between B7-1 and p75NTR, as compared to neurotrophin:p75NTR interface, the membrane-based presentation of B7-1, and the lack of detectable interaction with Sortilin family members, distinguish the B7-1 and neurotrophin interactions with p75NTR.

### Example 6: Mixed-Culture Assay to Evaluate B7-1 Induced Localization of PSD95 and MAP2 in Cultured Hippocampal Neurons

B7-1:p75NTR activity was confirmed using an orthogonal method for B7-1 presentation. Because interactions between p75NTR and B7-1 are likely to occur in trans between neurons and microglia, a mixed-culture assay system was developed that would more appropriately model the in vivo interaction. This type of mixed culture system has been used to evaluate assembly, due to the trans-synaptic signaling, or as assessed here, disassembly, of mature spines, by monitoring the expression of a transmembrane protein on heterologous cells which are then co-cultured with primary neurons.

As described herein, the B7-1 N82E mutant interacts with CD28, CTLA-4, and PD-L1, but has reduced binding to p75NTR (see FIG. 2; FIG. 3D). Stable HEK293 cell lines were generated that expressed one of B7-1, the B7-1 N82E mutant, or B7-2. Each of the B7-1, the B7-1 N82E mutant, or B7-2 proteins were C-terminally fused to mCherry. To ensure that the cells expressed similar levels of wild-type and mutant B7-1 and B7-2, the cells were sorted, and routinely evaluated by flow cytometry to ensure similar mCherry expression, and appropriate ligand binding.

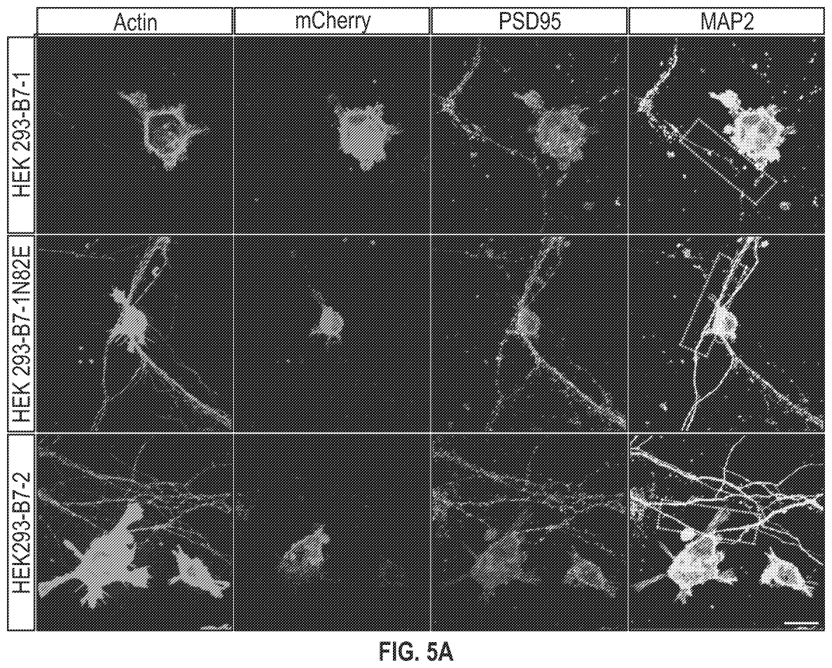

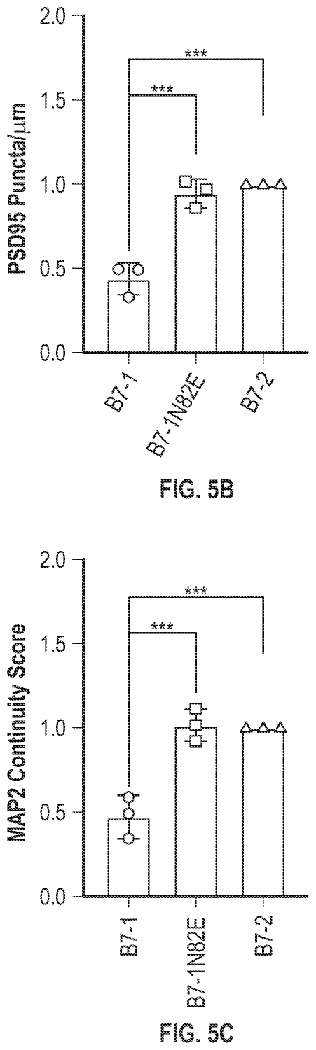

To determine how B7-1 expressing cells affect post-synaptic structures, stably transfected HEK293 cells were incubated with cultured primary DIV18 hippocampal mouse neurons for four hours. The cellular morphology was then analyzed by staining for actin, PSD95, and MAP2, and the cells were evaluated by confocal microscopy. Images of the hippocampal neurons co-cultured with HEK293 cells expressing B7-1, B7-1N82E, or B7-2 are shown in FIG. 5A. Dendrites in contact with B7-1-expressing cells had a significantly lower PSD95 density and lower MAP2 continuity than dendrites in direct contact with B7-2- or B7-1 N82E-expressing cells (FIG. 5B-5C). These results are consistent with the phenotypic changes observed upon addition of recombinant wild-type and mutant B7-1 protein. Most notably, dendrites of neurons incubated with B7-1 expressing cells exhibited a distinct punctate MAP2 staining pattern, while the dendrites of neurons incubated with B7-1N82E or B7-2 expressing cells exhibited more diffuse, uniform, and continuous distribution of MAP2 throughout the dendrite, which was phenotypically similar to untreated neurons (FIG. 5A).

### Example 7: Increased Expression of B7-1 and Activation of Microglia in CRND8 Mice

The CRND8 transgenic line model of Alzheimer's Disease (AD) was used in the experiments described in this example. The CRND8 transgenic line is a well characterized transgenic model that expresses a mutant amyloid precursor protein (APP) and that develops synaptic loss, gliosis and cognitive impairment within 6 months of birth.

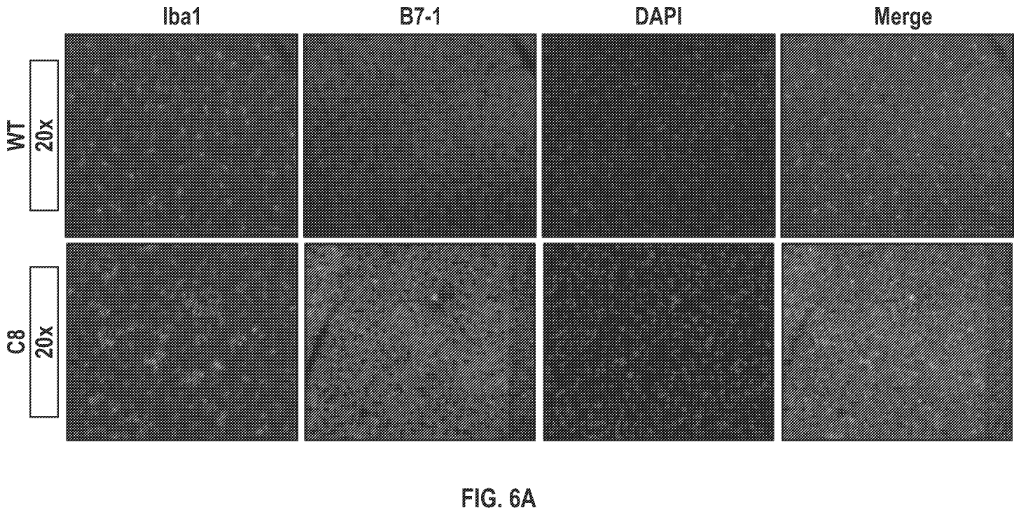

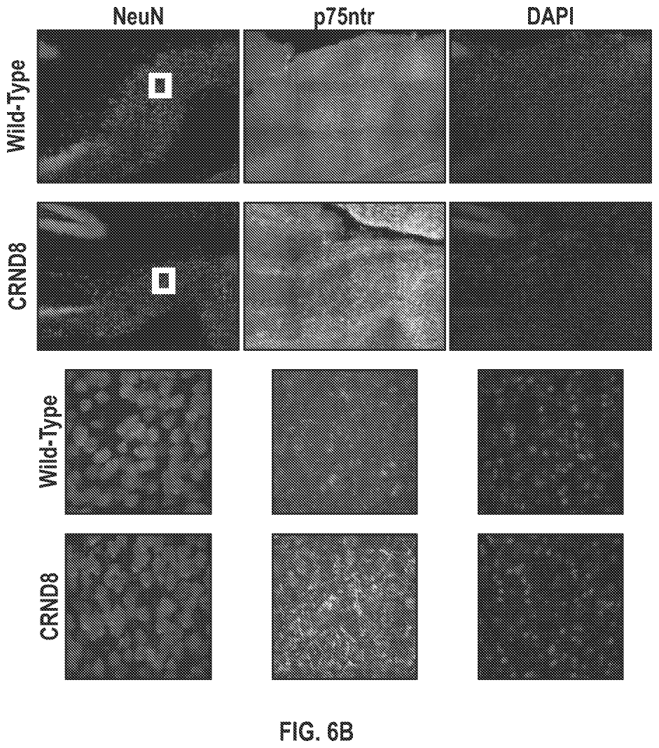

Brains from CRND8 transgenic mice or from wild type mice were evaluated for B7-1 expression and for the expression of the microglia marker Iba1. Increased Iba1 expression provided evidence of microglia with more extensive processes. Increased B7-1 expression was detected in the brains from CRND8 transgenic mice in 2.5 month-old mice when cognitive impairment and gliosis begins (data not shown) and in 7 month old mice when cognitive impairment and gliosis is progressing (FIG. 6).

As shown in FIG. 6A, more activated microglia and increased expression of B7-1 were detected in the CRND8 transgenic mouse brains than in the wild type mouse brains.

FIG. 6B shows that the sections of brains from a mouse model of Alzheimer's Disease (CRND8 transgenic mice, C8) exhibit increased expression of p75NTR compared to wild type mouse brains at 7 months.

### Example 8: Injection of B7-1 into the Mouse Hippocampal Subiculum Region Induces Pruning of Dendritic Spines In Vivo

The Example illustrates that injection of B7-1 causes loss of dendritic synaptic features.

C57Bl6 male mice (2.5 months) received 500 ng/ul of B7-1 or B7-2 into contralateral hemispheres of the subiculum region of the brains of wild type or. As a control saline was injected into the subiculum regions of control mice. Three hours post injection, mice were sacrificed and the brains were harvested. The harvested brains were then processed for Golgi analysis to quantitate the density of synaptic spines per length of either apical or basal dendrites.

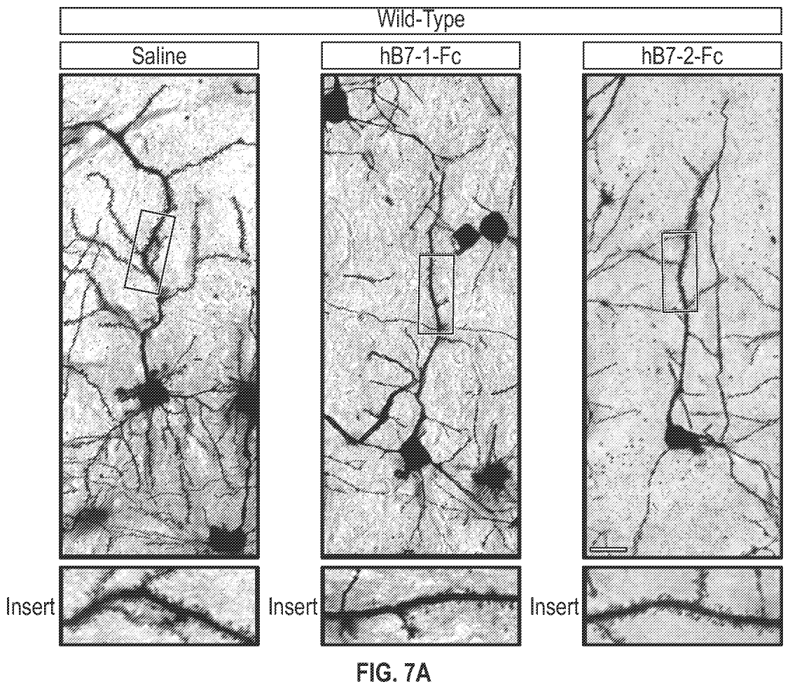

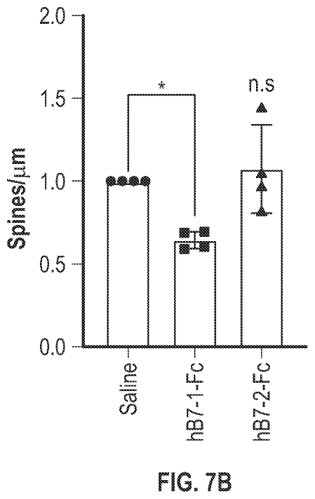

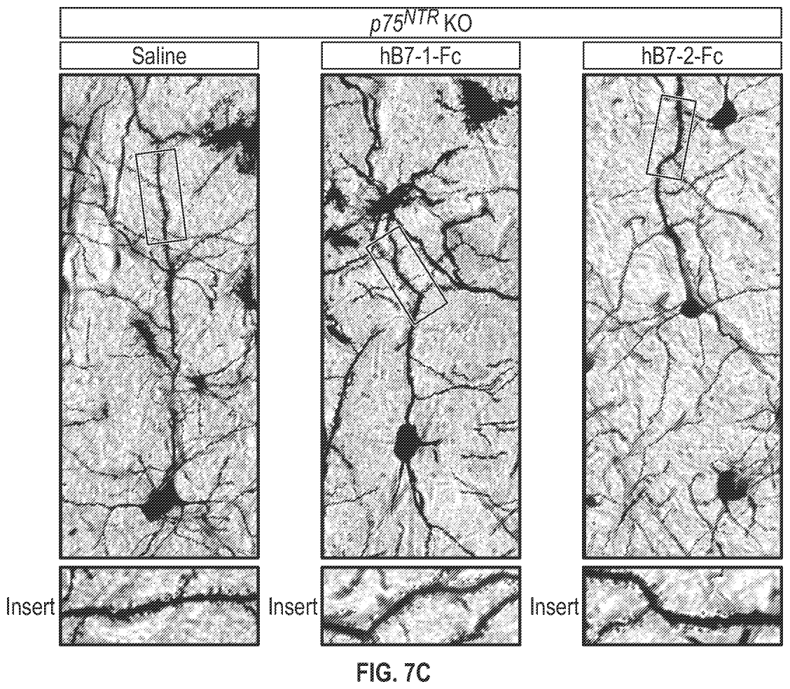

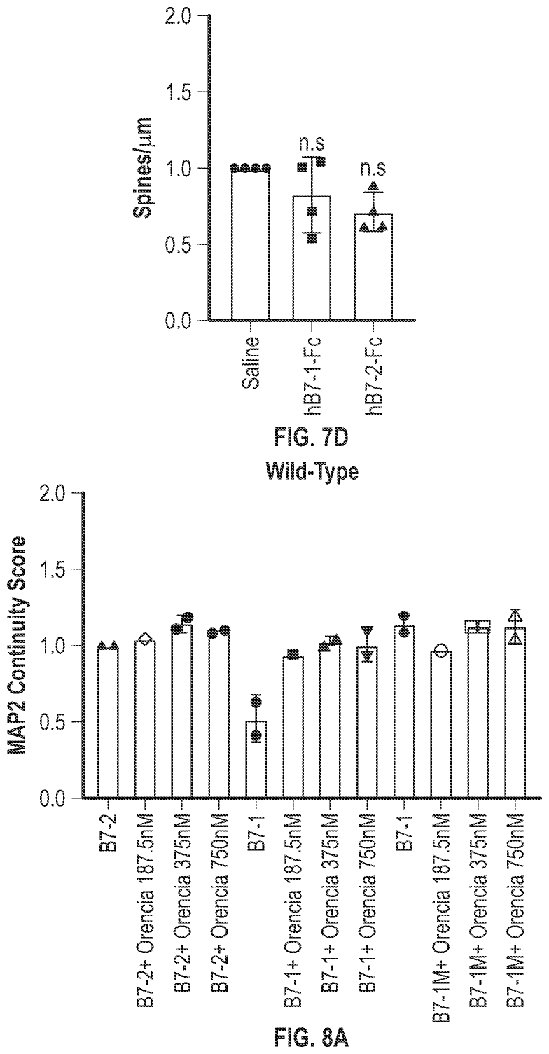

As shown in FIG. 7A-7B, direct injection of B7-1 into the subiculum of mice induces the pruning of dendritic spines in vivo. This result was not observed when B7-2 was injected into the subiculum of mice or when mice lacking p75NTR were injected with B7-1 (FIG. 7A-7D).

### Example 9: Orencia™ Blocks B7-1 Induced Loss of Synaptic Spines and Dendritic Microtubule Fragmentation

The Example illustrates that ORENCIA™ (abatacept) blocked the loos of synaptic spines and the dendritic microtubule fragmentation observed when neurons are cultured with B7-1. ORENCIA™ (abatacept) is a biologic used for the treatment of rheumatoid arthritis. It is an anti-inflammatory drug having the extracellular domain of CTLA-4 fused to a human IgG1 Fc fragment.

Mature hippocampal neurons were co-cultured with HEK293 cells expressing B7-1, or HEK293 cells expressing B7-2, in the presence of absence of ORENCIA™ (abatacept).

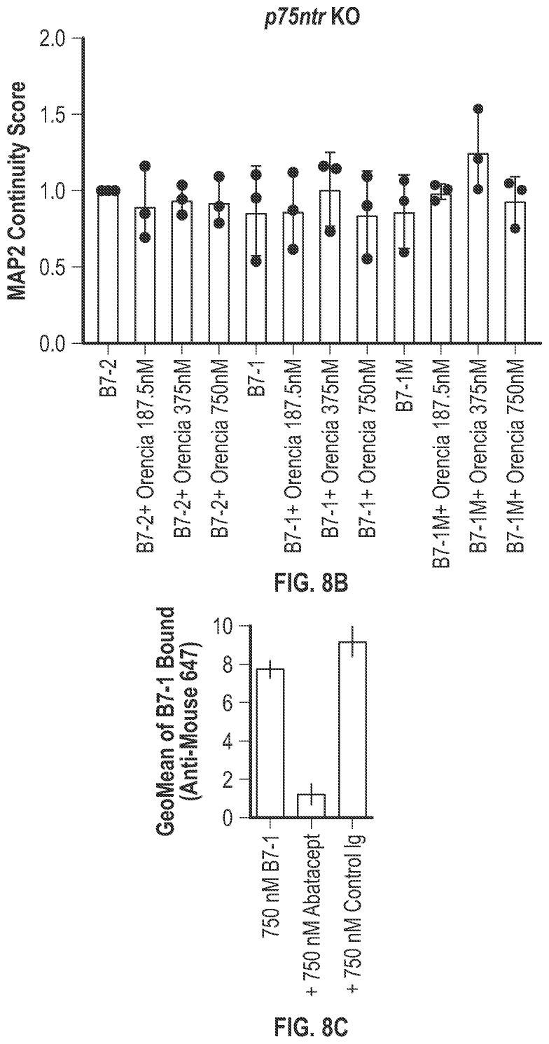

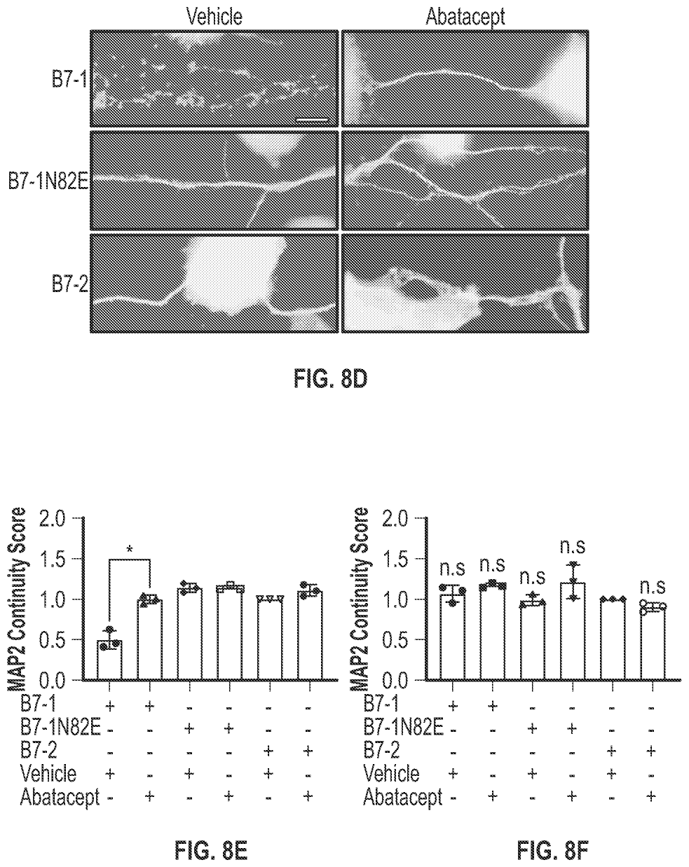

As shown in FIG. 8A, in neuronal cultures from wildtype mice, B7-1 induced MAP2 fragmentation in dendrites (lower MAP2 continuity score), but such MAP2 fragmentation was blocked by the addition of Orencia™ (abatacept). In neuronal cultures from p75 null mutant mice, B7-1 failed to induce MAP2 fragmentation, and Orencia™ had no effect (FIG. 8B). B7-2 did not induce MAP2 fragmentation, and hence the MAP2 continuity scores for neuronal cultures treated with B7-2 did not vary significantly when Orencia™ (abatacept) was included or when MAP2 fragmentation was measured in neuronal cultures from p75 null mutant mice (FIG. 8A-8B). Competition experiments using abatacept (Orencia™), also demonstrated that it efficiently blocked soluble B7-1 from binding to cells expressing p75 (FIG. 8C).

These studies indicate that abatacept was nontoxic to mature hippocampal neuron cultures. Moreover, these studies indicate that p75, CTLA-4 and abatacept all share overlapping recognition surfaces on B7-1.

In another study, abatacept (375 nM) was added to wild type hippocampal neurons co-cultured with either B7-1-expressing, B7-2-expressing, or B7-1 N82E-expressing HEK 293 cells, and the continuity of MAP2-positive processes was quantified. Abatacept inhibited the degeneration of MAP2-positive-processes of wild type neurons that were induced by B7-1 expressing cells (FIG. 8D-8E). As a control, studies were also performed using p75^{-/-} hippocampal neurons. No changes in MAP2 morphology were observed when abatacept was added to p75^{-/-} hippocampal neurons co-cultured with B7-protein expressing cells (FIG. 8F).

### Example 10: Generation of a Mouse Model Expressing a Chimeric Human:Mouse B7-1

The experimental results described herein show that primate B7-1, but not B7-1 from lower mammals, binds to p75. These results indicate that recent evolutionary substitutions in the ectodomain of human B7-1 confer binding. Human B7-1, however, binds to both human p75 and murine p75, which is consistent with the high evolutionary conservation of p75 across species.

The inventors sought to generate a knock-in mouse model expressing a chimeric human:mouse B7-1 (substituting human sequence in the ectodomain of murine B7-1), using CRISPR technology. Such a mouse is useful not only for evaluating the high evolutionary conservation of p75 across species but also as a model for identifying agents that can block B7-1 and p75 interactions.

CRISPR technology was used to generate a knock-in mouse model expressing a chimeric human:mouse B7-1 (substituting human sequence in the ectodomain of murine B7-1). First, the structural alignment of human and murine B7-1 was evaluated with a focus on comparison of the human and mouse B7-1 amino acids that interact with p75, as determined by the alanine scanning mutagenesis studies described above (Example 3; FIG. 2A-2C). Such a sequence alignment between a human B7-1 sequence (SEQ ID NO:1, HSq1) and a mouse B7-1 sequence (SEQ ID NO:5; MSq5) is shown below.

| | | |
|---|---|---|
| HSq1 | 42 | VKEVATLSCGHNVSVEELAQTRIYWQKEKKMVLTMMSGDMNIWPEYKNRTIFDITNNLSI |
| MSq5 | 46 | VKDKVLLPCRYNSPHEDESEDRIYWQKHDKVVLSVIAGKLKVWPEYKNRTLYDNTT-YSL |
| | | ** * * * * ****** * ** * ******** * * * |
| HSq1 | 102 | VILALRPSDEGTYECVVLKYEKDAFKREHLAEVTLSVKADFPTPSISDFEIPTSNIRRII |
| MSq5 | 105 | IILGLVLSDRGTYSCVVQKKERGTYEVKHLALVKLSIKADFSTPNITESGNPSADTKRIT |
| | | ** * ** *** *** * * *** * ** **** ** * * ** |
| HSq1 | 162 | CSTSGGFPEPHLSWLENGEELNAINTTVSQDPETELYAVSSKLDFNMTTNHSFMCLIKYG |
| MSq5 | 165 | CFASGGFPKPRFSWLENGRELPGINTTISQDPESELYTISSQLDENTTRNHTIKCLIKYG |
| | | * ***** * ****** ** **** ***** *** ** **** * ** ****** |
| HSq1 | 222 | HLRVNQTFNWNTTKQEHFPD-NLLPSWAITLISVNGIFVICCLTYCFAPRCRERRRNERL |
| MSq5 | 225 | DAHVSEDFTWEKPPEDPPDSKNTLVLFGAGFGAVITVVVIVVIIKCFCKHRSCFRRNEAS |
| | | * * * * * * ** ** **** |
| HSq1 | 281 | R |
| MSq5 | 285 | R |
| | | * |

The N-terminal IgV domain of B7-1 contains the amino acids which confer p75 binding and are divergent between the human and murine sequence. In examining the exon structure of B7-1 in each species, the inventors noted that murine exon 2 encodes 106 amino acids, and human exon 3 contains 105 amino acids of IgV domain (FIG. 9A). Hence, cDNAs were generated that encoded an entire substitution of the human (106 aa) for murine (105aa) sequence, or more limited substitutions within these exons. These cDNAs were incorporated into expression vector, and the constructed hybrid proteins were expressed in HEK 293 cells to evaluate protein stability and interaction with human and murine p75.

Only the cDNA encoding the entire exon substitution (human exon 2 substituted for murine exon 3) conferred p75 binding and maintained binding to CTLA-4 (FIG. 9B-9C) and CD28 (data not shown). A sequence for the human-mouse chimeric protein is shown below as SEQ ID NO:7

| | |
|---|---|
| 1 | MACNCQLMQD TPLLKFPCPR LILLFVLLIR LSQVSSGVIH |
| 41 | VTKEVKEVAT LSCGHNVSVE ELAQTRIYWQ KEKKMVLTMM |
| 81 | SGDMNIWPEY KNRTIFDITN NLSIVILALR PSDEGTYECV |
| 121 | VLKYEKDAFK REHLAEVTLS VKADFSTPNI TESGNPSADT |
| 161 | KRITCFASGG FPKPRFSWLE NGRELPGINT TISQDPESEL |
| 201 | YTISSQLDFN TTRNHTIKCL IKYGDAHVSE DFTWEKPPED |
| 241 | PPDSKNTLVL FGAGFGAVIT VVVIVVIIKC FCKHRSCFRR |
| 281 | NEASRETNNS LTFGPEEALA EQTVFL |

With this information, a chimeric knock-in mouse (h:mB7-1 KI) was generated using CRISPR (C57Bl6 mouse background, Einstein gene targeting facility). Two founder lines were obtained and sequenced to confirm the substituted sequence, and that inadvertent mutagenesis had not occurred. Founders from both lines have been further backcrossed to C57Bl6, and protein expression from spleen cells of the chimeric mice was confirmed by Western blot analysis (FIG. 9D).

### Example 11: B7-1 Targets p75 Neurotrophin Receptor in Neurodegeneration

B7-1 expression is upregulated on both monocyte and lymphocyte populations in response to immune activating stimuli. To evaluate hB7-1 induction in the chimeric knock-in mice (h:mB7-1 KI), splenocytes were harvested from wild type animals and from the h:mB7-1 animals after activation with either lipopolysaccharide (LPS) or anti-mCD3/anti-mCD28 antibodies. The splenocytes were stained with the following antibodies:
1) mCD45-APC (detecting all differentiated hematopoietic cells),
2) mCD3-APC Cy7 (detecting all T-lymphocytes),
3) mCD11b-Alexa700 (primarily detecting monocytes and neutrophils), and
4) either anti-mB7-1 Alexa488 or hB7-1 PE-Dazzle.
   After staining, the splenocytes were analyzed by flow cytometry.

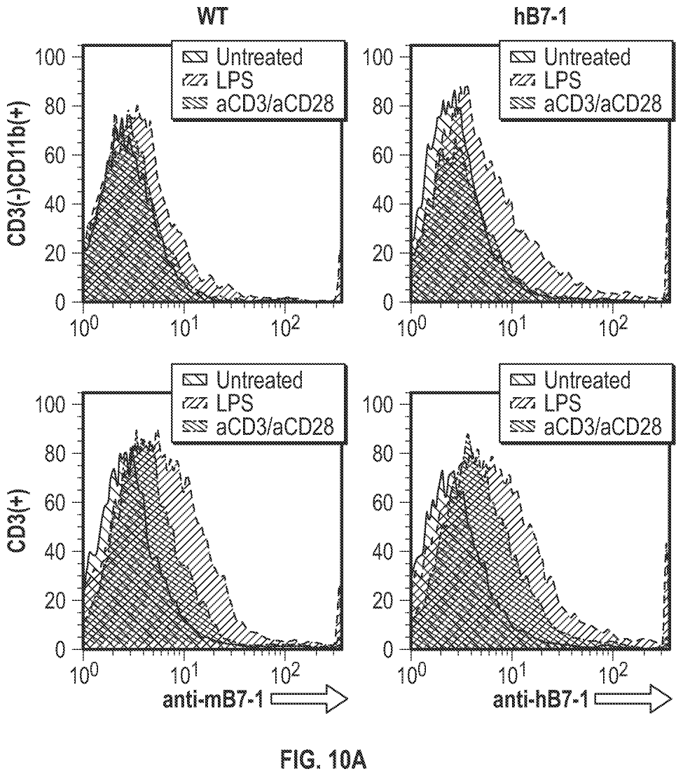

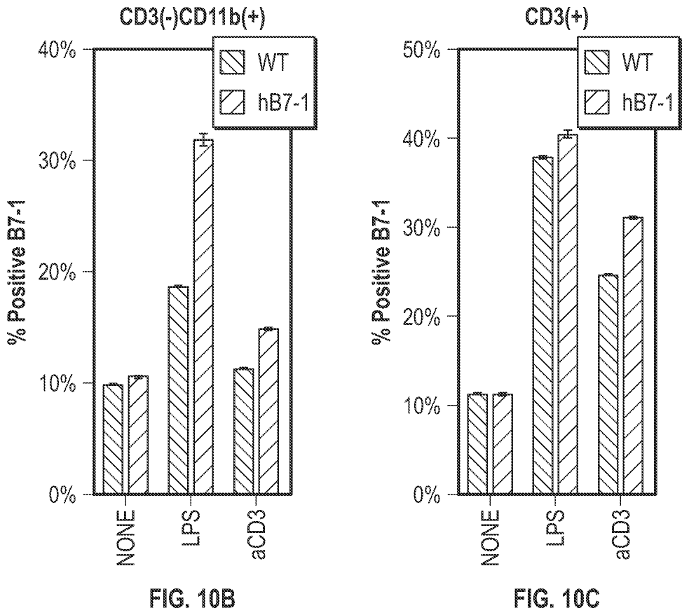

FIG. 10A-10C show that the cells were broadly gated into two populations. The cells in the first population (CD45(+)CD3(−)CD11b(+)) were primarily monocytes and neutrophils, while the cells in the second population (CD45(+)CD3(+)) were T-lymphocytes. LPS activation resulted in significant increases in B7-1 expression for both WT and hB7-1 populations analyzed. B7-1 expression was also induced using anti-CD3 and anti-CD28 antibodies, which crosslinks TCR and CD28 to trigger signaling that mirrors that observed during antigen-dependent activation. Importantly, the percent increase in hB7-1 expression was comparable to that of mB7-1, indicating that the h:mB7-1 mice express the hB7-1 chimeric protein in a way that mirrors WT animals and supports the use of these mice to study hB7-1 p75^{NTR} biology.

Collectively, the results described herein demonstrate that B7-1:p75 engagement is directly responsible for phenotypic changes in dendritic structure, and that a commercially available drug (abatacept) that impairs the binding of B7-1 to p75 can block acute negative remodeling of neurons. These studies provide proof of concept that drugs that impair the binding of B7-1 to p75 can block morphological changes to dendrite structure.

### REFERENCES

1 Ben-Shlomo, I., Yu Hsu, S., Rauch, R., Kowalski, H. W. & Hsueh, A. J. W. Signaling Receptome: A Genomic and Evolutionary Perspective of Plasma Membrane Receptors Involved in Signal Transduction. Science& #039;s STKE 2003, re9, doi:10.1126/stke.2003.187.re9 (2003).
2 Stamper, C. C. et al. Crystal structure of the B7-1/CTLA-4 complex that inhibits human immune responses. Nature 410, 608-611, doi:10.1038/35069118 (2001).
3 Gong, Y., Cao, P., Yu, H. J. & Jiang, T. Crystal structure of the neurotrophin-3 and p75NTR symmetrical complex. Nature 454, 789-793, doi:10.1038/nature07089 (2008).
4 Giza, J. I. et al. The BDNF Val66Met Prodomain Disassembles Dendritic Spines Altering Fear Extinction Circuitry and Behavior. Neuron 99, 163-178.e166, doi:10.1016/j.neuron.2018.05.024 (2018).
5 Sudhof, T. C. & Malenka, R. C. Understanding synapses: past, present, and future. Neuron 60, 469-476, doi:10.1016/j.neuron.2008.10.011 (2008).
6 Hong, S. et al. Complement and microglia mediate early synapse loss in Alzheimer mouse models. Science 352, 712-716, doi:10.1126/science.aad8373 (2016).
7 Sugiura, D. et al. Restriction of PD-1 function by cis-PDL1/CD80 interactions is required for optimal T cell responses. Science 364, 558, doi:10.1126/science.aav7062 (2019).
8 Williams, K., Ulvestad, E. & Antel, J. P. B7/BB-1 antigen expression on adult human microglia studied in vitro and in situ. Eur J Immunol 24, 3031-3037, doi:10.1002/eji.1830241217 (1994).
9 Awada, R. et al. Autotaxin downregulates LPS-induced microglia activation and proinflammatory cytokines production. J Cell Biochem 115, 2123-2132, doi:10.1002/jcb.24889 (2014).
10 Satoh, J., Lee, Y. B. & Kim, S. U. T-cell costimulatory molecules B7-1 (CD80) and B7-2 (CD86) are expressed in human microglia but not in astrocytes in culture. Brain Res 704, 92-96, doi:10.1016/0006-8993(95)01177-3 (1995).
11 Donat, C. K., Scott, G., Gentleman, S. M. & Sastre, M. Microglial Activation in Traumatic Brain Injury. Front Aging Neurosci 9, 208-208, doi:10.3389/fnagi.2017.00208 (2017).
12 Rawat, P. & Spector, S. A. Development and characterization of a human microglia cell model of HIV-1 infection. J Neurovirol 23, 33-46, doi:10.1007/s13365-016-0472-1 (2017).
13 Olson, J. K., Girvin, A. M. & Miller, S. D. Direct activation of innate and antigen presenting functions of microglia following infection with Theiler's virus. J Virol 75, 9780-9789, doi:10.1128/JVI.75.20.9780-9789.2001 (2001).
14 O'Keefe, G. M., Nguyen, V. T. & Benveniste, E. N. Regulation and function of class II major histocompatibility complex, CD40, and B7 expression in macrophages and microglia: Implications in neurological diseases. J Neurovirol 8, 496-512, doi:10.1080/13550280290100941 (2002).
15 Windhagen, A. et al. Expression of costimulatory molecules B7-1 (CD80), B7-2 (CD86), and interleukin 12 cytokine in multiple sclerosis lesions. J Exp Med 182, 1985-1996, doi:10.1084/jem.182.6.1985 (1995).
16 De Simone, R. et al. The costimulatory molecule 399 B7 is expressed on human microglia in culture and in multiple sclerosis acute lesions. J Neuropathol Exp Neurol 54, 175-187, doi:10.1097/00005072-199503000-00004 (1995).
17 Wolf, S. A., Gimsa, U., Bechmann, I. & Nitsch, R. Differential expression of costimulatory molecules B7-1 and B7-2 on microglial cells induced by Th1 and Th2 cells in organotypic brain tissue. Glia 36, 414-420, doi:10.1002/glia.1127 (2001).
18 Zinn, K. & Ozkan, E. Neural immunoglobulin superfamily interaction networks. Curr Opin Neurobiol 45, 99-105, doi:10.1016/j.conb.2017.05.010 (2017).
19 Johnson, D. et al. Expression and structure of the human NGF receptor. Cell 47, 545-554, doi:10.1016/0092-8674(86)90619-7 (1986).
20 Chao, M. V., Rajagopal, R. & Lee, F. S. Neurotrophin signaling in health and disease. Clin Sci (Lond) 110, 167-173, doi:10.1042/cs20050163 (2006).
21 Kraemer, B. R., Yoon, S. O. & Carter, B. D. The biological functions and signaling mechanisms of the p75 neurotrophin receptor. Handb Exp Pharmacol 220, 121-164, doi:10.1007/978-3-642-45106-5_6 (2014).
22 Dechant, G. & Barde, Y. A. The neurotrophin receptor p75(NTR): novel functions and implications for diseases of the nervous system. Nat Neurosci 5, 1131-1136, doi:10.1038/nn1102-1131 (2002).
23 Meeker, R. & Williams, K. The p75 neurotrophin receptor: at the crossroad of neural repair and death. Neural Regeneration Research 10, 721-725, doi:10.4103/1673-5374.156967 (2015).
24 Je, H. S. et al. Role of pro-brain-derived neurotrophic factor (proBDNF) to mature BDNF conversion in activity-dependent competition at developing neuromuscular synapses. Proceedings of the National Academy of Sciences of the United States of America 109, 15924-15929, doi:10.1073/pnas.1207767109 (2012).
25 Kisiswa, L., Fernandez-Suarez, D., Sergaki, M. C. & Ibanez, C. F. RIP2 Gates TRAF6 Interaction with Death Receptor p75(NTR) to Regulate Cerebellar Granule Neuron Survival. Cell Rep 24, 1013-1024, doi:10.1016/j.celrep.2018.06.098 (2018).
26 Bradshaw, J. M. et al. Monitoring signaling by the p75(NTR) receptor utilizing a caspase-3 activation assay amenable to small-molecule screening. Assay Drug Dev Technol 10, 353-364, doi:10.1089/adt.2011.398 (2012).
27 Lin, Z. et al. Structural basis of death domain signaling in the p75 neurotrophin receptor. Elife 4, e11692, doi:10.7554/eLife.11692 (2015).
28 Deinhardt, K. et al. Neuronal Growth Cone Retraction Relies on Proneurotrophin Receptor Signaling Through Rac. Science Signaling 4, ra82, doi:10.1126/scisignal.2002060 (2011).
29 Yan, R. et al. The Structure of the Pro-domain of Mouse proNGF in Contact with the NGF Domain. Structure (London, England: 1993) 27, 78-89.e73, doi:10.1016/j.str.2018.09.013 (2019).
30 Leloup, N., Chataigner, L. M. P. & Janssen, B. J. C. Structural insights into SorCS2—Nerve Growth Factor complex formation. Nature Communications 9, 2979, doi:10.1038/s41467-018-05405-z (2018).
31 Delivanoglou, N. et al. Spatio-temporal expression profile of NGF and the two-receptor system, TrkA and p75NTR, in experimental autoimmune encephalomyelitis. Journal of Neuroinflammation 17, 41, doi:10.1186/s12974-020-1708-9 (2020).
32 Ramani, S. R. et al. A secreted protein microarray platform for extracellular protein interaction discovery. Anal Biochem 420, 127-138, doi:10.1016/j.ab.2011.09.017 (2012).
33 Chaudhri, A. et al. PD-L1 Binds to B7-1 Only In Cis on the Same Cell Surface. Cancer Immunol Res 6, 921-929, doi:10.1158/2326-6066.Cir-17-0316 (2018).
34 Garrett-Thomson, S. C. et al. Mechanistic dissection of the PD-L1:B7-1 co-inhibitory immune complex. PloS one 15, e0233578, doi:10.1371/journal.pone.0233578 (2020).
35 Mischel, P. S. et al. The extracellular domain of p75NTR is necessary to inhibit neurotrophin-3 signaling through TrkA. J Biol Chem 276, 11294-11301, doi:10.1074/jbc.M005132200 (2001).
36 Liu, W. et al. Structural Basis of CD160:HVEM Recognition. Structure (London, England: 1993) 27, 1286-1295.e1284, doi:10.1016/j.str.2019.05.010 (2019).
37 Shrestha, R., Garrett, S. C., Almo, S. C. & Fiser, A. Computational Redesign of PD-1 Interface for PD-L1 Ligand Selectivity. Structure (London, England: 1993) 27, 829-836.e823, doi:10.1016/j.str.2019.03.006 (2019).
38 Ramagopal, U. A. et al. Structural basis for cancer immunotherapy by the first-in-class checkpoint inhibitor ipilimumab. Proceedings of the National Academy of Sciences of the United States of America 114, E4223-e4232, doi:10.1073/pnas.1617941114 (2017).
39 van der Merwe, P. A., Bodian, D. L., Daenke, S., Linsley, P. & Davis, S. J. CD80 (B7-1) binds both CD28 and CTLA-4 with a low affinity and very fast kinetics. J Exp Med 185, 393-403, doi:10.1084/jem.185.3.393 (1997).
40 Fraczkiewicz, R. & Braun, W. Exact and efficient analytical calculation of the accessible surface areas and their gradients for macromolecules. Journal of Computational Chemistry 19, 319-333 (1998).
41 Barron, A. M. S., Ho, J. D., Mantero, J. C., Bhawan, J. & Browning, J. L. Nerve growth factor receptor expression marks activated human perivascular adventitial fibroblasts in distinct perturbed states. The Journal of Immunology 202, 182.149 (2019).
42 Wang, S. et al. p75(NTR) mediates neurotrophin-induced apoptosis of vascular smooth muscle cells. Am J Pathol 157, 1247-1258, doi:10.1016/50002-9440(10)64640-8 (2000).
43 Bandola, J. et al. Neurotrophin Receptor p75NTR Regulates Immune Function of Plasmacytoid Dendritic Cells. Front Immunol 8, 981-981, doi:10.3389/fimmu.2017.00981 (2017).
44 Caporali, A. et al. Neurotrophin p75 receptor (p75NTR) promotes endothelial cell apoptosis and inhibits angiogenesis: implications for diabetes-induced impaired neovascularization in ischemic limb muscles. Circ Res 103, e15-e26, doi:10.1161/CIRCRESAHA.108.177386 (2008).
45 Biederer, T. & Scheiffele, P. Mixed-culture assays for analyzing neuronal synapse formation. Nat Protoc 2, 670-676, doi:10.1038/nprot.2007.92 (2007).
46 Sebastiani, A. et al. Proneurotrophin Binding to P75 Neurotrophin Receptor (P75ntr) Is Essential for Brain Lesion Formation and Functional Impairment after Experimental Traumatic Brain Injury. J Neurotrauma 32, 1599-1607, doi:10.1089/neu.2014.3751 (2015).
47 Kokaia, Z., Andsberg, G., Martinez-Serrano, A. & Lindvall, O. Focal cerebral ischemia in rats induces expression of p75 neurotrophin receptor in resistant striatal cholinergic neurons. Neuroscience 84, 1113-1125 (1998).
48 Magnus, T. et al. Microglial expression of the B7 family member B7 homolog 1 confers strong immune inhibition: implications for immune responses and autoimmunity in the CNS. J Neurosci 25, 2537-2546, doi:10.1523/jneurosci.4794-04.2005 (2005).
49 Menezes, S. M. et al. CD80+ and CD86+ B cells as biomarkers and possible therapeutic targets in HTLV-1 associated myelopathy/tropical spastic paraparesis and multiple sclerosis. J Neuroinflammation 11, 18, doi:10.1186/1742-2094-11-18 (2014).
50 Choi, S. & Friedman, W. J. Inflammatory cytokines IL-1beta and TNF-alpha regulate p75NTR expression in CNS neurons and astrocytes by distinct cell-type-specific signaling mechanisms. ASN Neuro 1, doi:10.1042/an20090009 (2009).
51 Ibanez, C. F. & Simi, A. p75 neurotrophin receptor signaling in nervous system injury and degeneration: paradox and opportunity. Trends Neurosci 35, 431-440, doi:10.1016/j.tins.2012.03.007 (2012).
52 Lee, K. F. et al. Targeted mutation of the gene encoding the low affinity NGF receptor p75 leads to deficits in the peripheral sensory nervous system. Cell 69, 737-749, doi:10.1016/0092-8674(92)90286-1 (1992).
53 Kaech, S. & Banker, G. Culturing hippocampal neurons. Nat Protoc 1, 2406-2415, doi:10.1038/nprot.2006.356 (2006).

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following Statements summarize aspects and features of the invention.

### Statements:

1. A method comprising incubating one or more test agents with B7-1 protein and p75 neurotrophin receptor and measuring whether one or more of the test agents reduces B7-1 binding to p75 neurotrophin receptor.
2. The method of statement 1, wherein one or more of the test agents is a small molecule, antibody, antibody fragment, antibody-derived construct, Fc-fusion protein, protein, peptide, aptamer, peptide aptamer, nucleic acid aptamer, darpin, nanobody, affinity reagent, liposome displaying at least one test agent, or cell expressing at least one test agent on its cell surface.
3. The method of statement 1 or 2, wherein the B7-1 or p75 neurotrophin receptor is in soluble form, for example, wherein the B7-1 or p75 neurotrophin receptor is fused to an Fc antibody region.
4. The method of any one of statements 1-3, wherein the B7-1 protein has a sequence comprising I36, T39, Y40, K43, S49, R63, Y65, N82, K120, Y121, K127, and K139.
5. The method of any one of statements 1-4, wherein the B7-1 protein has a sequence comprising at least 90% at least 85%, or at least 90%, or at least 95%, or at least 96%, or at least 97%, or at least 98%, or at least 99% sequence identity to SEQ ID NO:1.
6. The method of any one of statements 1-5, wherein the p75 neurotrophin receptor has a sequence comprising F136, S137, S137, E147, P150, P150, L165, and R182.
7. The method of any one of statements 1-6, wherein the p75 neurotrophin receptor has a sequence comprising at least 90% at least 85%, or at least 90%, or at least 95%, or at least 96%, or at least 97%, or at least 98%, or at least 99% sequence identity to SEQ ID NO:8.
8. The method of any one of statements 1-7, wherein the B7-1 and p75 neurotrophin receptor are expressed separately on different cells, or at least one of B7-1 or p75 neurotrophin receptor is expressed on a cell, or at least one of B7-1 or p75 is linked to a bead or carrier.
9. The method of any one of statements 1-8, further comprising selecting one or more of the test agents that reduce B7-1 binding to p75 neurotrophin receptor by at least 25%, or at least 50%, or at least 75% compared to a control assay mixture of the B7-1 and the p75 neurotrophin receptor without the one or more test agents, to thereby identify at least one B7-1 blocking agent.
10. The method of statement 9, further comprising incubating at least one B7-1 blocking agent with B7-1 in the presence of CD28 or CTLA-4, and measuring whether at least one of the B7-1 blocking agents reduces B7-1 binding to CD28 or CTLA-4.
11. The method of statement 10, further comprising selecting one or more of the B7-1 blocking agents that does not significantly reduce B7-1 binding to CD28 or CTLA-4 to thereby identify at least one B7-1-specific blocking agent.
12. The method of statement 11, further comprising incubating at least one B7-1-specific blocking agent in a culture comprising B7-1 (e.g., as soluble B7-1 or as cell-bound B7-1) and neurons that express p75 neurotrophin receptor, and measuring synaptic puncta density of the neurons that express p75 neurotrophin receptor.
13. The method of statement 12, further comprising selecting at least one B7-1-specific blocking agent that maintains higher levels of synaptic puncta density compared to a control culture comprising B7 (e.g., as soluble B7-1 or as cell-bound B7-1) and neurons that express p75 neurotrophin receptor without the B7-1-specific blocking agent, to thereby identify a B7-1 inhibitor.
14. The method of statement 12 or 13, wherein the neurons are dendritic cells.
15. The method of any one of statements 1-14, further comprising administering the B7-1 inhibitor or the B7-1-specific blocking agent to an animal with a neuronal condition or disease.
16. The method of statement 15, wherein the animal is a model animal having or exhibiting symptoms of Alzheimer's disease, cognitive impairment, multiple sclerosis, stroke, neuronal injury, pain (e.g., acute pain, chronic pain, neuropathic pain, nociceptive pain, radicular pain, thermal pain, or combinations thereof), traumatic neural injury, spinal cord injury, lupus, Parkinson's disease, anxiety, schizophrenia, manic depression, delirium, dementia, mental retardation, Huntington's disease, or Tourette's syndrome.
17. The method of statement 15 or 16, further comprising measuring whether the model animal has reduced symptoms of Alzheimer's disease, cognitive impairment, multiple sclerosis, stroke, neuronal injury, pain (e.g., acute pain, chronic pain, neuropathic pain, nociceptive pain, radicular pain, thermal pain, or combinations thereof), traumatic neural injury, spinal cord injury, lupus, Parkinson's disease, anxiety, schizophrenia, manic depression, delirium, dementia, mental retardation, Huntington's disease, or Tourette's syndrome, compared to a model animal that did not receive the at least one B7-1-specific blocking agent or at least one B7-1 inhibitor.
18. The method of statement 15, wherein the animal is a human having or exhibiting symptoms of Alzheimer's disease, cognitive impairment, multiple sclerosis, stroke, neuronal injury, traumatic neural injury, spinal cord injury, pain (e.g., acute pain, chronic pain, neuropathic pain, nociceptive pain, radicular pain, thermal pain, or combinations thereof), lupus, Parkinson's disease, anxiety, schizophrenia, manic depression, delirium, dementia, mental retardation, Huntington's disease, or Tourette's syndrome.
19. A method comprising administering abatacept, belatacept, a modified CTLA-4 protein that can block of B7 binding, a modified CD28 protein that can block of B7 binding, a modified Inducible T Cell Costimulator Ligand (ICOSL) protein that can block of B7 binding, or combinations thereof to a subject having a neuronal condition or disease to thereby treat the neuronal condition or disease.
20. The method of statement 19, wherein the neuronal condition or disease is Alzheimer's disease, cognitive impairment, multiple sclerosis, stroke, neuronal injury, traumatic neural injury, spinal cord injury, pain (e.g., acute pain, chronic pain, neuropathic pain, nociceptive pain, radicular pain, thermal pain, or combinations thereof), lupus, Parkinson's disease, anxiety, schizophrenia, manic depression, delirium, dementia, mental retardation, Huntington's disease, or Tourette's syndrome.
21. A modified p75 neurotrophin receptor protein comprising one or more replacements, deletions or insertions into a binding domain for B7-1 protein, nerve growth factor (NGF), proNGF, neurotrophin-3 (NT3), or receptor-type tyrosine-protein phosphatase F (PTPRF).
22. The modified p75 neurotrophin receptor protein of statement 21, comprising one or more mutations that reduce binding of the p75_{NTR} protein to B7-1.
23. The modified p75 neurotrophin receptor protein of statement 21 or 22, comprising one or more mutations at positions F136, S137, S137, E147, P150, P150, L165, or R182.
24. The modified p75 neurotrophin receptor protein of any one of statements 21-23, comprising one or more replacements, deletions, or insertions into amino acid positions corresponding to any or all of positions 36, 37, 38, 49, 95, 104, 136, 137, 147, 150, 162, 165, 171, or 182 of SEQ ID NO:8 (shown below).

| | |
|---|---|
| 1 | MGAGATGRAM DGPRLLLLLL LGVSLGGAKE ACPTGLYTHS |
| 41 | GECCKACNLG EGVAQPCGAN QTVCEPCLDS VTFSDVVSAT |
| 81 | EPCKPCTECV GLQSMSAPCV EADDAVCRCA YGYYQDETTG |
| 121 | RCEACRVCEA GSGLVFSCQD KQNTVCEECP DGTYSDEANH |
| 161 | VDPCLPCTVC EDTERQLREC TRWADAECEE IPGRWITRST |
| 201 | PPEGSDSTAP STQEPEAPPE QDLIASTVAG VVTTVMGSSQ |
| 241 | PVVTRGTTDN LIPVYCSILA AVVVGLVAYI AFKRWNSCKQ |
| 281 | NKQGANSRPV NQTPPPEGEK LHSDSGISVD SQSLHDQQPH |
| 321 | TQTASGQALK GDGGLYSSLP PAKREEVEKL LNGSAGDTWR |
| 361 | HLAGELGYQP EHIDSFTHEA CPVRALLASW ATQDSATLDA |
| 401 | LLAALRRIQR ADLVESLCSE STATSPV |

25. The modified p75 neurotrophin receptor protein of any one of statements 21-24, comprising one or more of F136D, S137A, S137D, E147D, P150A, P150D, L165A, or R182A.
26. The modified p75 neurotrophin receptor protein of any one of statements 21-24, comprising one or more of F136D, S137A, S137D, E147D, P150A, P150D, or L165A.
27. The modified p75 neurotrophin receptor protein of any one of statements 21-26, comprising one or more mutations, which reduce binding of the p75_{NTR} protein to PTPRF.
28. The modified p75 neurotrophin receptor protein of statement 27, comprising one or more mutations at positions L36, Y37, T38, L49 or R182 of the p75NTR protein, which reduce binding of the p75_{NTR} protein to PTPRF.
29. The modified p75 neurotrophin receptor protein of statement 27 or 28, comprising one or more L36D, Y37A, Y37D, T38D, L49D, or R182A mutations.
30. The modified p75 neurotrophin receptor protein of any one of statements 21-29, comprising one or more mutations which reduce p75 neurotrophin receptor binding with NGF, proNGF, or NT3.
31. The modified p75 neurotrophin receptor protein of any one of statements 21-30, comprising one or more mutations at positions L36, M95, D162, E171, or D104.
32. The modified p75 neurotrophin receptor protein of any one of statements 21-31, comprising one or more mutations at positions at L36D, M95A, D162A, D162R, E171A, D104R, which reduce or eliminate hydrogen or electrostatic bonds with NGF, proNGF, or NT3.
33. The modified p75 neurotrophin receptor protein of any one of statements 21-32, comprising a modified transmembrane domain.
34. The modified p75 neurotrophin receptor protein of statement 33, wherein the transmembrane domain is a hydrophobic domain at a position corresponding to amino acid positions 251-272 of SEQ ID NO:8.
35. The modified p75 neurotrophin receptor protein of statement 33 or 34, wherein the transmembrane domain has one or more replacements or deletions of one or more hydrophobic amino acids within amino acid positions corresponding to amino acid positions 251-272 or SEQ ID NO:8.
36. The modified p75 neurotrophin receptor protein of any one of statements 21-35, which is soluble p75 neurotrophin receptor protein that selectively binds B7-1, but does not bind NGF, proNGF, NT3, or PTPRF.
37. A composition comprising a carrier and the modified p75 neurotrophin receptor protein of any one of statements 21-36.
38. An expression cassette or expression vector comprising a promoter operably linked to a nucleic acid segment encoding the modified p75 neurotrophin receptor protein of any one of statements 21-37.
39. A host cell comprising the modified p75 neurotrophin receptor protein of any one of statements 21-36, or the expression cassette or expression vector of statement 38.
40. A composition comprising the expression cassette or expression vector of statement 37 or the host cell of statement 39.
41. A method comprising administering the modified p75 neurotrophin receptor protein of any of statements 21-36, the host cell of statement 39, the composition of statement 37 or 40, or the expression cassette or expression vector of statement 38 to a subject.
42. The method of statement 41, wherein the subject has a neuronal condition or disease.
43. The method of statement 42, wherein the neuronal condition or disease comprises pain (e.g., acute pain, chronic pain, neuropathic pain, nociceptive pain, radicular pain, thermal pain, or combinations thereof), cancer, anorexia, bulimia, asthma, Alzheimer's disease, lupus, Parkinson's disease, psychotic disorder, anxiety, schizophrenia, manic depression, delirium, dementia, mental retardation, dyskinesias, Huntington's disease, Tourette's syndrome, or a combination thereof.
44. An antibody comprising at least one cdr region that binds B7-1 or that comprises sequence identity to p75 neurotrophin receptor positions 36, 37, 38, 49, 95, 104, 136, 137, 147, 150, 162, 165, 171, or 182 of SEQ ID NO:8.
45. The antibody of statement 44, comprising at least one cdr region with sequence identity to positions 135-149, position 165, and position 182 of SEQ ID NO:8.
46. The antibody of statement 44 or 45, which binds B7-1.
47. The antibody of any one of statements 44-46, which is a human or humanized antibody.
48. A composition comprising carrier and the antibody of any one of statements 44-47.
49. A method comprising administering the antibody of any one of statements 44-47 to a subject having a neuronal condition or disease.
50. The method of statement 49, wherein the neuronal condition or disease comprises pain (e.g., acute pain, chronic pain, neuropathic pain, nociceptive pain, radicular pain, thermal pain, or combinations thereof), cancer, anorexia, bulimia, asthma, Alzheimer's disease, lupus, Parkinson's disease, psychotic disorder, anxiety, schizophrenia, manic depression, delirium, dementia, mental retardation, dyskinesias, Huntington's disease, Tourette's syndrome, or a combination thereof.
51. An antibody that binds to a p75 neurotrophin receptor peptide epitope, where the peptide epitope sequence includes 3-10 amino acids at any of p75 neurotrophin receptor positions 36, 37, 38, 49, 95, 104, 136, 137, 147, 150, 162, 165, 171, or 182 of SEQ ID NO:8.
52. An antibody comprising at least one cdr region that binds to a p75 neurotrophin receptor peptide epitope, where the peptide epitope spans 3-10 amino acids at any of p75 neurotrophin receptor positions 135-149, position 165, and position 182 of SEQ ID NO:8.
53. The antibody of any of statements 51 or 52, which binds p75 neurotrophin receptor.
54. The antibody of any of any one of statements 51-53, is a human, humanized, or mouse antibody.
55. A composition comprising carrier and the antibody of any of statements 51-54.
56. A modified B7-1 protein with one or more replacements, deletions or insertions in a binding domain for p75 neurotrophin receptor protein.
57. The modified B7-1 protein of statement 56, comprising one or more replacements, deletions, or insertions into amino acid positions corresponding to one or more of positions 36, 39, 40, 43, 49, 63, 65, 82, 120, 121, 127, or 139 of SEQ ID NO:1 (shown below)

| | |
|---|---|
| 1 | MGHTRRQGTS PSKCPYLNFF QLLVLAGLSH FCSGVIHVTY |
| 41 | EVKEVATLSC GHNVSVEELA QTRIYWQKEK KMVLTMMSGD |
| 81 | MNIWPEYKNR TIFDITNNLS IVILALRPSD EGTYECVVLK |
| 121 | YEKDAFKREH LAEVTLSVKA DFPTPSISDF EIPTSNIRRI |
| 161 | ICSTSGGFPE PHLSWLENGE ELNAINTTVS QDPETELYAV |
| 201 | SSKLDFNMTT NHSFMCLIKY GHLRVNQTFN WNTTKQEHFP |
| 241 | DNLLPSWAIT LISVNGIFVI CCLTYCFAPR CRERRRNERL |
| 281 | RRESVRPV. |

58. The modified B7-1 protein of statement 56 or 57, comprising one or more of I36D, T39A, Y40D, K43D, S49A, R63D, Y65A, N82E, K120D, Y121D, K127D, or K139D modifications.
59. The modified B7-1 protein of any one of statements 56-58, further comprising a modified transmembrane domain.
60. The modified B7-1 protein of statement 59, wherein the transmembrane domain is a hydrophobic domain at a position corresponding to amino acid positions 243-263 of SEQ ID NO:1.
61. The modified B7-1 protein of statement 59 or 60, wherein the transmembrane domain has one or more replacements or deletions of one or more hydrophobic amino acids within amino acid positions corresponding to positions 243-263 of SEQ ID NO:1.
62. The modified B7-1 protein of any of statements 56-61, which has at least one mutation in the B7-1 cytosolic domain.
63. A composition comprising a carrier and the modified B7-1 protein of any of statements 56-61.
64. An expression cassette or expression vector comprising a heterologous promoter operably linked to a nucleic acid segment encoding the modified B7-1 protein of any of statements 56-61.
65. A host cell comprising the modified B7-1 protein of any of statements 56-61, or the expression cassette or expression vector of statement 64.
66. A method comprising administering the modified B7-1 protein of statement 56-61, the composition of statement 63, the expression cassette or expression vector of statement 64, or the host cell of statement 65 to a subject.
67. The methods of statement 66, wherein the subject has a neuronal condition or disease.
68. The method of statement 67, wherein the neuronal condition or disease comprises pain (e.g., acute pain, chronic pain, neuropathic pain, nociceptive pain, radicular pain, thermal pain, or combinations thereof), cancer, anorexia, bulimia, asthma, Alzheimer's disease, lupus, Parkinson's disease, psychotic disorder, anxiety, schizophrenia, manic depression, delirium, dementia, mental retardation, dyskinesias, Huntington's disease, Tourette's syndrome, or a combination thereof.
69. An antibody comprising at least one cdr region with sequence identity to at least three of positions 36, 39, 40, 43, 49, 63, 65, 82, 120, 121, 127, or 139 of SEQ ID NO:1.
70. The antibody of statement 69, which binds p75 neurotrophin receptor.
71. The antibody of statement 69 or 70, which is a human or humanized antibody.
72. An antibody comprising at least one cdr region that binds to a B7-1 peptide epitope, where the peptide epitope has 3-10 amino acids at any of B7-1 positions 36, 39, 40, 43, 49, 63, 65, 82, 120, 121, 127, or 139 of SEQ ID NO:1.
73. The antibody of statement 72, which is a human or humanized antibody.
74. A composition comprising the antibody of any one of statements 69-73.
75. A method comprising administering the antibody of any one of statements 69-73, or the composition of statement 74, to a subject.
76. A peptide that comprises an 8-30 amino acid sequence comprising LSRKIGRT (SEQ ID NO:11) or LSRKAVRRA (SEQ ID NO:12) that inhibits B7-1:p75 binding.
77. The peptide of statement 76, wherein the peptide also inhibits binding of p75_{NTR} to NT3, NGF, proNGF, neurotrophin, or a combination thereof.
78. A composition comprising a carrier and the peptide of statement 76 or 77.
79. The method, modified B7-1 protein, modified p75 neurotrophin receptor, antibody, peptide or composition of any one of statements 1-78, wherein “inhibits” or “inhibiting” or “inhibition” is at least 25%, or at least 50%, or at least 60%, or at least 75%, or at least 80%, or at least 85%, or at least 90%, or at least 95%, or at least 96%, or at least 97%, or at least 98%, or at least 99% inhibition.
80. The method, modified B7-1 protein, modified p75 neurotrophin receptor, antibody, peptide or composition of statement 79, wherein the “inhibits” or “inhibiting” or “inhibition” is compared to a control.
81. The method, modified B7-1 protein, modified p75 neurotrophin receptor, antibody, peptide or composition of statement 80, wherein control is the quantity of B7-1:p75 neurotrophin receptor binding without one or more of the test agents, modified B7-1 proteins, modified p75 neurotrophin receptors, antibodies, peptides or compositions.
82. The modified B7-1 protein, modified p75 neurotrophin receptor, antibody, peptide or composition of any one of statements 1-78 as a medicament for the treatment of a neuronal condition or disease.
83. A B7-1 human-mouse chimera protein comprising a sequence comprising at least 95% sequence identity to SEQ ID NO:7.
84. An animal expressing a B7-1 human-mouse chimera protein comprising a sequence comprising at least 95% sequence identity to SEQ ID NO:7.
85. The animal of statement 84, that expresses the B7-1 human-mouse chimera protein instead of the animal's endogenous B7-1 protein.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, reference to “a nucleic acid” or “a protein” or “a cell” includes a plurality of such nucleic acids, proteins, or cells (for example, a solution or dried preparation of nucleic acids or expression cassettes, a solution of proteins, or a population of cells). In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

1. A method comprising incubating one or more test agents with B7-1 and p75 neurotrophin receptor and measuring whether one or more of the test agents reduces B7-1 binding to p75 neurotrophin receptor.

2. The method of claim 1, wherein one or more of the test agents is a small molecule, antibody, antibody fragment, antibody-derived construct, Fc-fusion protein, protein, peptide, aptamer, peptide aptamer, nucleic acid aptamer, darpin, nanobody, affinity reagent, liposome displaying at least one test agent, or cell expressing at least one test agent on its cell surface.

3. The method of claim 1, wherein the B7-1 or the p75 neurotrophin receptor is in soluble form.

4. The method of claim 1, wherein the B7-1 or the p75 neurotrophin receptor is fused to an Fc antibody fragment.

5. The method of claim 1, wherein the B7-1 and the p75 neurotrophin receptor are expressed separately on different cells, or at least one of B7-1 or p75 neurotrophin receptor is linked to different beads or carriers.

6. The method of claim 1, further comprising selecting one or more of the test agents that reduce B7-1 binding to p75 neurotrophin receptor by at least 25%, or at least 50%, or at least 75% compared to a control assay mixture of the B7-1 and the p75 neurotrophin receptor without the one or more test agents, to thereby identify at least one B7-1 blocking agent.

7. The method of claim 1, further comprising selecting one or more of the test agents that reduce B7-1 binding to p75 neurotrophin receptor and also reduces B7-1 binding to CD28, CTLA-4, or both.

8. The method of claim 6, further comprising incubating at least one B7-1 blocking agent with B7-1 in the presence of CD28 or CTLA-4, and measuring whether at least one of the B7-1 blocking agents reduces B7-1 binding to CD28 or CTLA-4.

9. The method of claim 8, further comprising selecting one or more of the B7-1 blocking agents that does not significantly reduce B7-1 binding to CD28 or CTLA-4 to thereby identify at least one B7-1-specific blocking agent.

10. The method of claim 9, further comprising incubating at least one B7-1-specific blocking agent in a culture comprising B7-1 and neurons that express p75 neurotrophin receptors, and measuring synaptic puncta density of the neurons that express p75 neurotrophin receptor.

11. The method of claim 10, wherein the neurons comprise dendrites.

12. The method of claim 10, further comprising selecting at least one B7-1-specific blocking agent that maintains higher levels of synaptic puncta density compared to a control culture comprising B7-1 and neurons that express p75 neurotrophin receptor without the B7-1-specific blocking agent, to thereby identify a B7-1 inhibitor.

13. The method of claim 12, further comprising administering the B7-1 inhibitor to an animal model of a neuronal condition or disease.\

14. The method of claim 13, further comprising measuring whether the animal model has reduced symptoms of Alzheimer's disease, cognitive impairment, multiple sclerosis, stroke, neuronal injury, traumatic neural injury, spinal cord injury, pain, lupus, Parkinson's disease, anxiety, schizophrenia, manic depression, delirium, dementia, mental retardation, Huntington's disease, or Tourette's syndrome, compared to a model animal that did not receive the at least one B7-1-specific blocking agent or at least one B7-1 inhibitor.

15. A method comprising administering abatacept or belatacept to a subject having a neuronal condition or disease to thereby treat the neuronal condition or disease.

16. The method of claim 15, wherein the subject has symptoms of Alzheimer's disease, cognitive impairment, multiple sclerosis, stroke, neuronal injury, traumatic neural injury, spinal cord injury, pain, lupus, Parkinson's disease, anxiety, schizophrenia, manic depression, delirium, dementia, mental retardation, Huntington's disease, or Tourette's syndrome.

17. A method comprising administering a modified CTLA-4 protein that can block of B7 binding, a modified CD28 protein that can block of B7 binding, a modified Inducible T Cell Costimulator Ligand (ICOSL) protein that can block of B7 binding, or combinations thereof to a subject having a neuronal condition or disease to thereby treat the neuronal condition or disease.

18-37. (canceled)


SEQUENCE LISTING (12 sequences)

SEQ ID NO: 1 - protein, 280 aa, Homo sapiens
MGHTRRQGTSPSKCPYLNFFQLLVLAGLSHFCSGVIHVTYEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERL

SEQ ID NO: 2 - protein, 18 aa, Homo sapiens
LLPSWAITLISVNGIFVI

SEQ ID NO: 3 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 4 - DNA, 2737 bp, Homo sapiens
aaaccctctgtaaagtaacagaagttagaaggggaaatgtcgcctctctgaagattacccaaagaaaaagtgatttgtcattgctttatagactgtaagaagagaacatctcagaagtggagtcttaccctgaaatcaaaggatttaaagaaaaagtggaatttttcttcagcaagctgtgaaactaaatccacaacctttggagacccaggaacaccctccaatctctgtgtgttttgtaaacatcactggagggtcttctacgtgagcaattggattgtcatcagccctgcctgttttgcacctgggaagtgccctggtcttacttgggtccaaattgttggctttcacttttgaccctaagcatctgaagccatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataacagtgtccgcagaagcaaggggctgaaaagatctgaaggtcccacctccatttgcaattgacctcttctgggaacttcctcagatggacaagattaccccaccttgccctttacgtatctgctcttaggtgcttcttcacttcagttgctttgcaggaagtgtctagaggaatatggtgggcacagaagtagctctggtgaccttgatcaaggtgttttgaaatgcagaattcttgagttctggaagggactttagagaataccagtgttattaatgacaaaggcactgaggcccagggaggtgacccgaattataaaggccagcgccagaacccagatttcctaactctggtgctctttccctttatcagtttgactgtggcctgttaactggtatatacatatatatgtcaggcaaagtgctgctggaagtagaatttgtccaataacaggtcaacttcagagactatctgatttcctaatgtcagagtagaagattttatgctgctgtttacaaaagcccaatgtaatgcataggaagtatggcatgaacatctttaggagactaatggaaatattattggtgtttacccagtattccatttttttcattgtgttctctattgctgctctctcactcccccatgaggtacagcagaaaggagaactatccaaaactaatttcctctgacatgtaagacgaatgatttaggtacgtcaaagcagtagtcaaggaggaaagggatagtccaaagacttaactggttcatattggactgataatctctttaaatggctttatgctagtttgacctcatttgtaaaatatttatgagaaagttctcatttaaaatgagatcgttgtttacagtgtatgtactaagcagtaagctatcttcaaatgtctaaggtagtaactttccatagggcctccttagatccctaagatggctttttctccttggtatttctgggtctttctgacatcagcagagaactggaaagacatagccaactgctgttcatgttactcatgactcctttctctaaaactgccttccacaattcactagaccagaagtggacgcaacttaagctgggataatcacattatcatctgaaaatctggagttgaacagcaaaagaagacaacatttctcaaatgcacatctcatggcagctaagccacatggctgggatttaaagcctttagagccagcccatggctttagctacctcactatgctgcttcacaaaccttgctcctgtgtaaaactatattctcagtgtagggcagagaggtctaacaccaacataaggtactagcagtgtttcccgtattgacaggaatacttaactcaataattcttttcttttccatttagtaacagttgtgatgactatgtttctattctaagtaattcctgtattctacagcagatactttgtcagcaatactaagggaagaaacaaagttgaaccgtttctttaataa

SEQ ID NO: 5 - protein, 306 aa, Mus musculus
MACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL

SEQ ID NO: 6 - DNA, 2688 bp, Mus musculus
gagttttatacctcaatagactcttactagtttctctttttcaggttgtgaaactcaaccttcaaagacactctgttccatttctgtggactaataggatcatctttagcatctgccgggtggatgccatccaggcttctttttctacatctctgtttctcgatttttgtgagcctaggaggtgcctaagctccattggctctagattcctggctttccccatcatgttctccaaagcatctgaagctatggcttgcaattgtcagttgatgcaggatacaccactcctcaagtttccatgtccaaggctcattcttctctttgtgctgctgattcgtctttcacaagtgtcttcagatgttgatgaacaactgtccaagtcagtgaaagataaggtattgctgccttgccgttacaactctcctcatgaagatgagtctgaagaccgaatctactggcaaaaacatgacaaagtggtgctgtctgtcattgctgggaaactaaaagtgtggcccgagtataagaaccggactttatatgacaacactacctactctcttatcatcctgggcctggtcctttcagaccggggcacatacagctgtgtcgttcaaaagaaggaaagaggaacgtatgaagttaaacacttggctttagtaaagttgtccatcaaagctgacttctctacccccaacataactgagtctggaaacccatctgcagacactaaaaggattacctgctttgcttccgggggtttcccaaagcctcgcttctcttggttggaaaatggaagagaattacctggcatcaatacgacaatttcccaggatcctgaatctgaattgtacaccattagtagccaactagatttcaatacgactcgcaaccacaccattaagtgtctcattaaatatggagatgctcacgtgtcagaggacttcacctgggaaaaacccccagaagaccctcctgatagcaagaacacacttgtgctctttggggcaggattcggcgcagtaataacagtcgtcgtcatcgttgtcatcatcaaatgcttctgtaagcacagaagctgtttcagaagaaatgaggcaagcagagaaacaaacaacagccttaccttcgggcctgaagaagcattagctgaacagaccgtcttcctttagttcttctctgtccatgtgggatacatggtattatgtggctcatgaggtctcatctaccatttgcaactgacctcttctgcaaaggacttcagaaacctagcactaccctggctctgcaaacacggttctctaggtgaagcctctgcagtggtttgcagaagtactcagacgaatgaaccacagtagttctgctgattctgaggacgtagtttagagactgaattctttggaaaggacatagggacgatttgcacatttgcttgcacatcacacacacacacacacacacacacacacacacacccacacacacacactctctctcgataccttaggatagggttctaccctgttgctcagtgacaaagaatcactctgtggcggaggcaggcttcaagcttgcagcaatcctcctgcaccagtttcctgagtgccagacttccaggtgtaagctatggcacttagcagaacactagctgaatcaatgaagacactgaggttccaagagggaacctgaattatgaaggtgagtcagaatccagatttcctggctctaccactcttaacctgtatctgttagaccccaagctctgagctcatagacaagctaatttaaaatgcttttaataagcagaaggctcagttagtacggggttcaggatactgcttactggcaatatttgactagcctctattttgtttgttttttaaggcctactgactgtagtgtaatttgtaggaaacatgttgctatgtatacccatttgagggtaataaaaatgttggtaattttcagccagcactttccaggtatttccctttttatccttcatgtccttaaaagaaaccagagtcaaccagaattaactgcttttgttgtctaagatgaatgtatgccttttatacttcaaagcaaaagtcaagttggaggcacagttcgtaacttactcgttcatattgaacttataatgaatgtctttaggcaactttgcccttttacacagttcttgatatagttcttatttcaaatgagattatttactgagtaatcatctgtcctcacataaacaacttaagtgatactagccttacacagaacttcctcagattcctggaagtggcttttgctctttggtatagatctgctacagtggaattgaaaaataaaatgtacgttagacacacttgcctgtaatccctgcactcagtatactgaggctgaagaactgaattccaggtagcatggcctggatagtaagatcctgtctgaaacccactcctccatcaatgtgccccaaagaaatggaaaacaagaagcagctaactgctgcttctatactgaaggttcccttttctctgagatgtaatccacaacctacaagaccagcagtgagcttaagtcaagacaatcagatattctagtctgaatttataggaggacatcgaatggcagccaaacttctcaacgggaccagaatctgttgtatgcccaggaaacaggtccatttttatgtgggtg

SEQ ID NO: 7 - protein, 306 aa, Artificial Sequence
  A synthetic sequence
MACNCQLMQDTPLLKFPCPRLILLFVLLIRLSQVSSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL

SEQ ID NO: 8 - protein, 427 aa, Homo sapiens
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSCKQNKQGANSRPVNQTPPPEGEKLHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV

SEQ ID NO: 9 - protein, 20 aa, Homo sapiens
LIPVYCSILAAVVVGLVAYI

SEQ ID NO: 10 - DNA, 3408 bp, Homo sapiens
agagcgagccgagccgcggccagctccggcgggcagggggggcgctggagcgcagcgcagcgcagccccatcagtccgcaaagcggaccgagctggaagtcgagcgctgccgcgggaggcgggcgatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtggaacagctgcaagcagaacaagcaaggagccaacagccggccagtgaaccagacgcccccaccagagggagaaaaactccacagcgacagtggcatctccgtggacagccagagcctgcatgaccagcagccccacacgcagacagcctcgggccaggccctcaagggtgacggaggcctctacagcagcctgcccccagccaagcgggaggaggtggagaagcttctcaacggctctgcgggggacacctggcggcacctggcgggcgagctgggctaccagcccgagcacatagactcctttacccatgaggcctgccccgttcgcgccctgcttgcaagctgggccacccaggacagcgccacactggacgccctcctggccgccctgcgccgcatccagcgagccgacctcgtggagagtctgtgcagtgagtccactgccacatccccggtgtgagcccaaccggggagcccccgccccgccccacattccgacaaccgatgctccagccaacccctgtggagcccgcacccccaccctttggggggggcccgcctggcagaactgagctcctctgggcaggacctcagagtccaggccccaaaaccacagccctgtcagtgcagcccgtgtggccccttcacttctgaccacacttcctgtccagagagagaagtgcccctgctgcctccccaaccctgcccctgccccgtcaccatctcaggccacctgcccccttctcccacactgctaggtgggccagcccctcccaccacagcaggtgtcatatatggggggccaacaccagggatggtactagggggaagtgacaaggccccagagactcagagggaggaatcgaggaaccagagccatggactctacactgtgaacttggggaacaagggtggcatcccagtggcctcaaccctccctcagcccctcttgccccccaccccagcctaagatgaagaggatcggaggcttgtcagagctgggaggggttttcgaagctcagcccacccccctcattttggatataggtcagtgaggcccagggagaggccatgattcgcccaaagccagacagcaacggggaggccaagtgcaggctggcaccgccttctctaaatgaggggcctcaggtttgcctgagggcgaggggagggtggcaggtgaccttctgggaaatggcttgaagccaagtcagctttgccttccacgctgtctccagacccccaccccttccccactgcctgcccacccgtggagatgggatgcttgcctagggcctggtccatgatggagtcaggtttggggttcgtggaaagggtgctgcttccctctgcctgtccctctcaggcatgcctgtgtgacatcagtggcatggctccagtctgctgccctccatcccgacatggacccggagctaacactggcccctagaatcagcctaggggtcagggaccaaggacccctcaccttgcaacacacagacacacgcacacacacacacaggaggagaaatctcacttttctccatgagttttttctcttgggctgagactggatactgcccggggcagctgccagagaagcatcggagggaattgaggtctgctcggccgtcttcactcgcccccgggtttggcgggccaaggactgccgaccgaggctggagctggcgtctgtcttcaagggcttacacgtggaggaatgctcccccatcctccccttccctgcaaacatggggttggctgggcccagaaggttgtgatgaagaaaagtgggccagtgtgggaatgcggcaagaaggaattgacttcgactgtgacctgtggggatttctcccagctctagacaaccctgcaaaggactgttttttcctgagcttggccagaagggggccatgaggcctcagtggactttccaccccctccctggcctgttctgttttgcctgaagttggagtgagtgtggctcccctctatttagcatgacaagccccaggcaggctgtgcgctgacaaccaccgctccccagcccagggttcccccagccctgtggaagggactaggagcactgtagtaaatggcaattctttgacctcaacctgtgatgaggggaggaaactcacctgctggcccctcacctgggcacctggggagtgggacagagtctgggtgtatttattttcctccccagcaggtggggagggggtttgggggcttgcaagtatgttttagcatgtgtttggttctggggcccctttttactccccttgagctgagatggaacccttttggcccccgagctgggggccatgagctccagacccccagcaaccctcctatcacctcccctccttgcctcctgtgtaatcatttcttgggccctcctgaaacttacacacaaaacgttaagtgatgaacattaaatagcaaagaaagaaaaa

SEQ ID NO: 11 - protein, 8 aa, Homo sapiens
LSRKIGRT

SEQ ID NO: 12 - protein, 9 aa, Homo sapiens
LSRKAVRRA